(19) United States
(12) Patent Application Publication
(10) Pub. No.: US 2020/0405685 A1
(43) Pub. Date: Dec. 31, 2020

(54) HIGH CANNABIGEROL CANNABIS PLANTS, METHODS OF PRODUCING AND METHODS OF USING THEM

(71) Applicant: Biotech Institute, LLC, Westlake Village, CA (US)

(72) Inventors: Mark A. LEWIS, Westlake Village, CA (US); Steven HABA, Westlake Village, CA (US)

(21) Appl. No.: 16/770,795

(22) PCT Filed: Dec. 7, 2018
(86) PCT No.: PCT/US2018/064549

§ 371 Date: Jun. 8, 2020

Related U.S. Application Data
(60) Provisional application No. 62/596,561, filed on Dec. 8, 2017.
(60) Provisional application No. 62/696,066, filed on Jul. 10, 2018.

(51) Int. Cl. A61K 31/352; A61K 31/05; A61K 36/185; A01H 6/28

(52) U.S. Cl. CPC A61K 31/352; A01H 6/28; A61K 36/185; A61K 31/05

(57) ABSTRACT

The invention provides compositions and methods for the breeding, production, processing and use of Specialty Cannabis and oannabinoid compositions. The Specialty Cannabis plants, plant parts, plant tissues and plant cells of the present disclosure comprise high levels of CBG in combination with one or more other cannabinoids.

### CROSS REFERENCE TO RELATED APPLICATIONS

The current application claims the benefit of priority to U.S. Provisional Application Ser. 62/696,066, filed on Jul. 10, 2018, and U.S. Provisional Application Ser. No. 62/596,561, filed Dec. 8, 2017, which is hereby incorporated by reference in its entirety.

### FIELD OF THE INVENTION

The invention relates to Specialty Cannabis plants with high cannabigerol contents, compositions and methods for making and using said cannabis plants and compositions.

### BACKGROUND OF THE INVENTION

Cannabis, more commonly known as marijuana, is a genus of flowering plants that includes at least three species, Cannabis sativa, Cannabis indica, and Cannabis ruderalis as determined by plant phenotypes and secondary metabolite profiles. In practice however, cannabis nomenclature is often used incorrectly or interchangeably. Cannabis literature can be found referring to all cannabis varieties as “sativas” or all cannabinoid-producing plants as “indicas”. Indeed the promiscuous crosses of indoor cannabis breeding programs have made it difficult to distinguish varieties; with most cannabis being sold in the United States, having features of both sativa and indica species.

Modern classification methods of cannabis plants now rely on the chemical phenotypes of cannabis inflorescences to categorize plants in a manner that provides meaningful information about the plant's expected organoleptic and medicinal effects. One of the major factors in classifying a new cannabis strain is the plant's cannabinoid profile. Best known for its production of Δ⁹-tetrahydrocannabinol (THC), and Δ⁹-tetrahydrocannabinolic acid (THCA), cannabis plants have actually been reported to produce at least 85 different cannabinoids. Surveys of analyzed cannabis inflorescences however, show that almost all known cannabis varieties available today have been bred to produce high levels of THC, at the expense of other cannabinoid constituents.

There thus remains a need for novel cannabis varieties expressing additional cannabinoids with individual or synergistic recreational and medicinal applications. The present invention addresses some of the shortcomings of the prior art by providing for Specialty Cannabis plants with novel cannabinoid profiles providing for improved recreational and medicinal effects.

### SUMMARY OF THE INVENTION

According to the methods and compositions of the present invention, plants, plant parts, plant tissues and plant cells are produced to contain novel and useful combinations of cannabinoids with improved recreational and medicinal effects.

In some embodiments, the Specialty Cannabis plants, plant parts, plant tissues and plant cells of the present disclosure comprise high levels of CBG in combination with one or more other cannabinoids.

In some embodiments, the present disclosure teaches a cannabis plant, or an asexual clone of said cannabis plant, or a plant part, tissue, or cell thereof, which is capable of producing a female inflorescence, said inflorescence comprising: a) a functional B_{T} allele b) a cannabigerol (CBG max) content of at least 2.0% by weight c) a non-CBG max cannabinoid content of at least 5.0% by weight, wherein the contents of all cannabinoids are measured by high performance liquid chromatography (HPLC) and calculated based on dry weight of the inflorescence; wherein a representative sample of seed producing said plant has been deposited under NCIMB Nos. 43257, 43261, 43263, and 43264.

In some embodiments, the cannabis plant, or an asexual clone of said cannabis plant, or a plant part, tissue, or cell thereof of the present disclosure does not comprise a functional BD allele.

In some embodiments, the cannabis plant, or an asexual clone of said cannabis plant, or a plant part, tissue, or cell thereof of the present disclosure comprises a terpene oil content greater than about 1.0% by weight; wherein the terpene oil content is the additive content of terpinolene, alpha phellandrene, beta ocimene, carene, limonene, gamma terpinene, alpha pinene, alpha terpinene, beta pinene, fenchol, camphene, alpha terpineol, alpha humulene, beta caryophyllene, linalool, caryophyllene oxide, and myrcene as measured by GC-FID and calculated based on dry weight of the inflorescence.

In some embodiments, the cannabis plant, or an asexual clone of said cannabis plant, or a plant part, tissue, or cell thereof of the present disclosure comprises a terpene oil content greater than about 1.5% by weight.

In some embodiments, the cannabis plant, or an asexual clone of said cannabis plant, or a plant part, tissue, or cell thereof of the present disclosure comprises a terpene oil content greater than about 2.0% by weight.

In some embodiments, the cannabis plant, or an asexual clone of said cannabis plant, or a plant part, tissue, or cell thereof of the present disclosure comprises a CBG max content of at least 3% by weight as measured by HPLC and calculated based on dry weight of the inflorescence.

In some embodiments, the cannabis plant, or an asexual clone of said cannabis plant, or a plant part, tissue, or cell thereof of the present disclosure comprises a CBG max content of at least 4% by weight as measured by HPLC and calculated based on dry weight of the inflorescence.

In some embodiments, the cannabis plant, or an asexual clone of said cannabis plant, or a plant part, tissue, or cell thereof of the present disclosure comprises a CBG max content of at least 5% by weight as measured by HPLC and calculated based on dry weight of the inflorescence.

In some embodiments, the cannabis plant, or an asexual clone of said cannabis plant, or a plant part, tissue, or cell thereof of the present disclosure comprises a CBG max content of at least 6% by weight as measured by HPLC and calculated based on dry weight of the inflorescence.

In some embodiments, the cannabis plant, or an asexual clone of said cannabis plant, or a plant part, tissue, or cell thereof of the present disclosure comprises a CBG max content of at least 7% by weight as measured by HPLC and calculated based on dry weight of the inflorescence.

In some embodiments, the cannabis plant, or an asexual clone of said cannabis plant, or a plant part, tissue, or cell thereof of the present disclosure comprises a CBG max content of at least 8% by weight as measured by HPLC and calculated based on dry weight of the inflorescence.

In some embodiments, the cannabis plant, or an asexual clone of said cannabis plant, or a plant part, tissue, or cell thereof of the present disclosure comprises a non-CBG max cannabinoid content that is at least 7.5% by weight as measured by HPLC and calculated based on dry weight of the inflorescence.

In some embodiments, the cannabis plant, or an asexual clone of said cannabis plant, or a plant part, tissue, or cell thereof of the present disclosure comprises a non-CBG max cannabinoid content that is at least 10.0% by weight as measured by HPLC and calculated based on dry weight of the inflorescence.

In some embodiments, the cannabis plant, or an asexual clone of said cannabis plant, or a plant part, tissue, or cell thereof of the present disclosure comprises a non-CBG max cannabinoid content that is at least 12.5% by weight as measured by HPLC and calculated based on dry weight of the inflorescence.

In some embodiments, the cannabis plant, or an asexual clone of said cannabis plant, or a plant part, tissue, or cell thereof of the present disclosure comprises a Terpene Profile in which myrcene is not the dominant terpene; wherein the Terpene Profile is defined as terpinolene, alpha phellandrene, beta ocimene, carene, limonene, gamma terpinene, alpha pinene, alpha terpinene, beta pinene, fenchol, camphene, alpha terpineol, alpha humulene, beta caryophyllene, linalool, caryophyllene oxide, and myrcene.

In some embodiments, the cannabis plant, or an asexual clone of said cannabis plant, or a plant part, tissue, or cell thereof of the present disclosure comprises a Terpene Profile in which the first or second most abundant terpene in the Terpene Profile is terpinolene.

In some embodiments, the cannabis plant, or an asexual clone of said cannabis plant, or a plant part, tissue, or cell thereof of the present disclosure comprises a Terpene Profile in which the first or second most abundant terpene in the Terpene Profile is alpha phellandrene.

In some embodiments, the cannabis plant, or an asexual clone of said cannabis plant, or a plant part, tissue, or cell thereof of the present disclosure comprises a Terpene Profile in which the first or second most abundant terpene in the Terpene Profile is carene.

In some embodiments, the cannabis plant, or an asexual clone of said cannabis plant, or a plant part, tissue, or cell thereof of the present disclosure comprises a Terpene Profile in which the first or second most abundant terpene in the Terpene Profile is limonene.

In some embodiments, the cannabis plant, or an asexual clone of said cannabis plant, or a plant part, tissue, or cell thereof of the present disclosure comprises a Terpene Profile in which the first or second most abundant terpene in the Terpene Profile is gamma terpinene.

In some embodiments, the cannabis plant, or an asexual clone of said cannabis plant, or a plant part, tissue, or cell thereof of the present disclosure comprises a Terpene Profile in which the first or second most abundant terpene in the Terpene Profile is alpha pinene.

In some embodiments, the cannabis plant, or an asexual clone of said cannabis plant, or a plant part, tissue, or cell thereof of the present disclosure comprises a Terpene Profile in which the first or second most abundant terpene in the Terpene Profile is alpha terpinene.

In some embodiments, the cannabis plant, or an asexual clone of said cannabis plant, or a plant part, tissue, or cell thereof of the present disclosure comprises a Terpene Profile in which the first or second most abundant terpene in the Terpene Profile is beta pinene.

In some embodiments, the cannabis plant, or an asexual clone of said cannabis plant, or a plant part, tissue, or cell thereof of the present disclosure comprises a Terpene Profile in which the first or second most abundant terpene in the Terpene Profile is fenchol.

In some embodiments, the cannabis plant, or an asexual clone of said cannabis plant, or a plant part, tissue, or cell thereof of the present disclosure comprises a Terpene Profile in which the first or second most abundant terpene in the Terpene Profile is camphene.

In some embodiments, the cannabis plant, or an asexual clone of said cannabis plant, or a plant part, tissue, or cell thereof of the present disclosure comprises a Terpene Profile in which the first or second most abundant terpene in the Terpene Profile is alpha terpineol.

In some embodiments, the cannabis plant, or an asexual clone of said cannabis plant, or a plant part, tissue, or cell thereof of the present disclosure comprises a Terpene Profile in which the first or second most abundant terpene in the Terpene Profile is alpha humulene.

In some embodiments, the cannabis plant, or an asexual clone of said cannabis plant, or a plant part, tissue, or cell thereof of the present disclosure comprises a Terpene Profile in which the first or second most abundant terpene in the Terpene Profile is beta caryophyllene.

The cannabis plant, or an asexual clone of said cannabis plant, or a plant part, tissue, or cell thereof of claim 15, wherein the first or second most abundant terpene in the Terpene Profile is linalool.

In some embodiments, the cannabis plant, or an asexual clone of said cannabis plant, or a plant part, tissue, or cell thereof of the present disclosure comprises a Terpene Profile in which the first or second most abundant terpene in the Terpene Profile is caryophyllene oxide.

In some embodiments, the cannabis plant, or an asexual clone of said cannabis plant, or a plant part, tissue, or cell thereof of the present disclosure comprises a Terpene Profile in which the first or second most abundant terpene in the Terpene Profile is beta ocimene.

In some embodiments, the cannabis plant, or an asexual clone of said cannabis plant, or a plant part, tissue, or cell thereof of the present disclosure comprises a Terpene Profile in which the first or second most abundant terpene in the Terpene Profile is myrcene.

In some embodiments, the present disclosure teaches a cannabis extract from the cannabis plant, plant part, tissue, or cell of any one of the disclosed Specialty Cannabis.

In some embodiments, the cannabis extract of the present disclosure is selected from the group consisting of kief, hashish, bubble hash, solvent reduced oils, sludges, e-juice, and tinctures.

In some embodiments, the cannabis extract of the present disclosure comprises greater than 25% CBG max content and greater than 10% non-CBG max cannabinoid content as measured by HPLC and based on weight of the extract.

In some embodiments, the present disclosure teaches a method of breeding cannabis plants with high CBG max and non-CBG max cannabinoid contents, said method comprising: (i) making a cross between a first cannabis plant of claim 1, and a second cannabis plant to produce an F1 plant; (ii) harvesting the resulting seed; (iii) growing said seed; and (iv) selecting for the desired phenotypes, wherein the resulting selected cannabis plant is comprises at least 2.0% CBG max, and at least 5.0% non-CBG max cannabinoid content.

In some embodiments, the present disclosure teaches a method of producing cannabis plants cannabis plants high CBG max and non-CBG max cannabinoid contents, said method comprising: (i) obtaining a cannabis seed, or cutting from a first cannabis plant of claim 1; (ii) placing said cannabis seed or cutting in an environment conducive to plant growth; (iii) allowing said cannabis seed or cutting to produce a cannabis plant; (iv) selecting for the desired phenotypes; wherein the resulting selected cannabis plant is comprises at least 2.0% CBG max, and at least 5.0% non-CBG max cannabinoid content.

In some embodiments, the present disclosure teaches a cannabis female inflorescence comprising: a) a functional B_{T} allele; b) a cannabigerol (CBG max) content of at least 2.0% by weight; c) a non-CBG max cannabinoid content of at least 5.0% by weight, wherein the contents of all cannabinoids are measured by high performance liquid chromatography (HPLC) and calculated based on dry weight of the inflorescence; wherein a representative sample of seed producing said inflorescence has been deposited under NCIMB Nos. 43257, 43261, 43263, and 43264.

In some embodiments, the inflorescence of the present disclosure does not comprise a functional BD allele.

In some embodiments, the inflorescence of the present disclosure comprises a terpene oil content greater than about 1.0% by weight; wherein the terpene oil content is the additive content of terpinolene, alpha phellandrene, beta ocimene, carene, limonene, gamma terpinene, alpha pinene, alpha terpinene, beta pinene, fenchol, camphene, alpha terpineol, alpha humulene, beta caryophyllene, linalool, caryophyllene oxide, and myrcene as measured by GC-FID and calculated based on dry weight of the inflorescence.

In some embodiments, the inflorescence of the present disclosure comprises a terpene oil content greater than about 1.5% by weight.

In some embodiments, the inflorescence of the present disclosure comprises a terpene oil content greater than about 2.0% by weight.

In some embodiments, the inflorescence of the present disclosure comprises a CBG max content of at least 3% by weight as measured by HPLC and calculated based on dry weight of the inflorescence.

In some embodiments, the inflorescence of the present disclosure comprises a CBG max content of at least 4% by weight as measured by HPLC and calculated based on dry weight of the inflorescence.

In some embodiments, the inflorescence of the present disclosure comprises a CBG max content of at least 5% by weight as measured by HPLC and calculated based on dry weight of the inflorescence.

In some embodiments, the inflorescence of the present disclosure comprises a CBG max content of at least 6% by weight as measured by HPLC and calculated based on dry weight of the inflorescence.

In some embodiments, the inflorescence of the present disclosure comprises a CBG max content of at least 7% by weight as measured by HPLC and calculated based on dry weight of the inflorescence.

In some embodiments, the inflorescence of the present disclosure comprises a CBG max content of at least 8% by weight as measured by HPLC and calculated based on dry weight of the inflorescence.

In some embodiments, the inflorescence of the present disclosure comprises a non-CBG max cannabinoid content that is at least 7.5% by weight as measured by HPLC and calculated based on dry weight of the inflorescence.

In some embodiments, the inflorescence of the present disclosure comprises a non-CBG max cannabinoid content that is at least 10.0% by weight as measured by HPLC and calculated based on dry weight of the inflorescence.

In some embodiments, the inflorescence of the present disclosure comprises a non-CBG max cannabinoid content that is at least 12.5% by weight as measured by HPLC and calculated based on dry weight of the inflorescence.

In some embodiments, the inflorescence of the present disclosure comprises a Terpene Profile in which myrcene is not the dominant terpene; wherein the Terpene Profile is defined as terpinolene, alpha phellandrene, beta ocimene, carene, limonene, gamma terpinene, alpha pinene, alpha terpinene, beta pinene, fenchol, camphene, alpha terpineol, alpha humulene, beta caryophyllene, linalool, caryophyllene oxide, and myrcene.

In some embodiments, the inflorescence of the present disclosure comprise a Terpene Profile in which the first or second most abundant terpene in the Terpene Profile is terpinolene.

In some embodiments, the inflorescence of the present disclosure comprise a Terpene Profile in which the first or second most abundant terpene in the Terpene Profile is alpha phellandrene.

In some embodiments, the inflorescence of the present disclosure comprise a Terpene Profile in which the first or second most abundant terpene in the Terpene Profile is carene.

In some embodiments, the inflorescence of the present disclosure comprise a Terpene Profile in which the first or second most abundant terpene in the Terpene Profile is limonene.

In some embodiments, the inflorescence of the present disclosure comprise a Terpene Profile in which the first or second most abundant terpene in the Terpene Profile is gamma terpinene.

In some embodiments, the inflorescence of the present disclosure comprise a Terpene Profile in which the first or second most abundant terpene in the Terpene Profile is alpha pinene.

In some embodiments, the inflorescence of the present disclosure comprise a Terpene Profile in which the first or second most abundant terpene in the Terpene Profile is alpha terpinene.

In some embodiments, the inflorescence of the present disclosure comprise a Terpene Profile in which the first or second most abundant terpene in the Terpene Profile is beta pinene.

In some embodiments, the inflorescence of the present disclosure comprise a Terpene Profile in which the first or second most abundant terpene in the Terpene Profile is fenchol.

In some embodiments, the inflorescence of the present disclosure comprise a Terpene Profile in which the first or second most abundant terpene in the Terpene Profile is camphene.

In some embodiments, the inflorescence of the present disclosure comprise a Terpene Profile in which the first or second most abundant terpene in the Terpene Profile is terpineol.

In some embodiments, the inflorescence of the present disclosure comprise a Terpene Profile in which the first or second most abundant terpene in the Terpene Profile is alpha humulene.

In some embodiments, the inflorescence of the present disclosure comprise a Terpene Profile in which the first or second most abundant terpene in the Terpene Profile is beta caryophyllene.

In some embodiments, the inflorescence of the present disclosure comprise a Terpene Profile in which the first or second most abundant terpene in the Terpene Profile is linalool.

In some embodiments, the inflorescence of the present disclosure comprise a Terpene Profile in which the first or second most abundant terpene in the Terpene Profile is caryophyllene oxide.

In some embodiments, the inflorescence of the present disclosure comprise a Terpene Profile in which the first or second most abundant terpene in the Terpene Profile is beta ocimene.

In some embodiments, the inflorescence of the present disclosure comprise a Terpene Profile in which the first or second most abundant terpene in the Terpene Profile is myrcene.

In yet other embodiments, the present disclosure teaches a cannabis plant, or an asexual clone of said cannabis plant, or a plant part, tissue, or cell thereof, which is capable of producing a female inflorescence, said inflorescence comprising: a) a cannabigerol (CBG max) content of at least 2.0% by weight; b) a tetrahydrocannabinol (THC max) and cannabidiol (CBD max) combined content of at least 5.0%, wherein the contents of all cannabinoids are measured by high performance liquid chromatography (HPLC) and calculated based on dry weight of the inflorescence; wherein a representative sample of seed producing said plant has been deposited under NCIMB Nos. 43257, 43261, 43263, and 43264.

In some embodiments, the cannabis plant, or an asexual clone of said cannabis plant, or a plant part, tissue, or cell thereof of the present disclosure comprises less than 1% CBD max.

In some embodiments, the cannabis plant, or an asexual clone of said cannabis plant, or a plant part, tissue, or cell thereof of the present disclosure comprises a combined terpene oil content of terpinolene, alpha phellandrene, beta ocimene, carene, limonene, gamma terpinene, alpha pinene, alpha terpinene, beta pinene, fenchol, camphene, alpha terpineol, alpha humulene, beta caryophyllene, linalool, caryophyllene oxide, and myrcene of at least 1.0%, as measured by GC-FID and calculated based on dry weight of the inflorescence.

In some embodiments, the cannabis plant, or an asexual clone of said cannabis plant, or a plant part, tissue, or cell thereof of the present disclosure comprises a combined terpene oil content greater than about 1.5% by weight.

In some embodiments, the cannabis plant, or an asexual clone of said cannabis plant, or a plant part, tissue, or cell thereof of the present disclosure comprises a combined terpene oil content greater than about 2.0% by weight.

In some embodiments, the cannabis plant, or an asexual clone of said cannabis plant, or a plant part, tissue, or cell thereof of the present disclosure comprises a CBG max content of at least 3% by weight as measured by HPLC and calculated based on dry weight of the inflorescence.

In some embodiments, the cannabis plant, or an asexual clone of said cannabis plant, or a plant part, tissue, or cell thereof of the present disclosure comprises a CBG max content of at least 4% by weight as measured by HPLC and calculated based on dry weight of the inflorescence.

In some embodiments, the cannabis plant, or an asexual clone of said cannabis plant, or a plant part, tissue, or cell thereof of the present disclosure comprises a CBG max content of at least 5% by weight as measured by HPLC and calculated based on dry weight of the inflorescence.

In some embodiments, the cannabis plant, or an asexual clone of said cannabis plant, or a plant part, tissue, or cell thereof of the present disclosure comprises a tetrahydrocannabinol (THC max) and cannabidiol (CBD max) combined content that is at least 7.5% by weight as measured by HPLC and calculated based on dry weight of the inflorescence.

In some embodiments, the cannabis plant, or an asexual clone of said cannabis plant, or a plant part, tissue, or cell thereof of the present disclosure comprises a tetrahydrocannabinol (THC max) and cannabidiol (CBD max) combined content that is at least 10.0% by weight as measured by HPLC and calculated based on dry weight of the inflorescence.

In some embodiments, the cannabis plant, or an asexual clone of said cannabis plant, or a plant part, tissue, or cell thereof of the present disclosure comprises a tetrahydrocannabinol (THC max) and cannabidiol (CBD max) combined content that is at least 12.5% by weight as measured by HPLC and calculated based on dry weight of the inflorescence.

In some embodiments, the present disclosure teaches a composition comprising: a) a cannabigerol (CBG max) content of at least 20% by weight; and b) a non-CBG max cannabinoid content of at least 30.0% by weight, wherein the contents of all cannabinoids are measured by high performance liquid chromatography (HPLC) and calculated based on weight of the composition.

In some embodiments, the composition of the present disclosure comprises a terpene oil content greater than about 5% by weight; wherein the terpene oil content is the additive content of terpinolene, alpha phellandrene, beta ocimene, carene, limonene, gamma terpinene, alpha pinene, alpha terpinene, beta pinene, fenchol, camphene, alpha terpineol, alpha humulene, beta caryophyllene, linalool, caryophyllene oxide, and myrcene as measured by GC-FID and calculated based on weight of the composition.

In some embodiments, the composition of the present disclosure comprises a terpene oil content greater than about 8% by weight.

In some embodiments, the composition of the present disclosure comprises a terpene oil content greater than about 10.0% by weight.

In some embodiments, the composition of the present disclosure comprises a CBG max content of at least 30% by weight as measured by HPLC and calculated based on weight of the composition.

In some embodiments, the composition of the present disclosure comprises a CBG max content of at least 40% by weight as measured by HPLC and calculated based on weight of the composition.

In some embodiments, the composition of the present disclosure comprises a CBG max content of at least 50% by weight as measured by HPLC and calculated based on dry weight of the inflorescence.

In some embodiments, the composition of the present disclosure comprises a CBG max content of at least 60% by weight as measured by HPLC and calculated based on dry weight of the inflorescence.

In some embodiments, the composition of the present disclosure comprises a CBG max content of at least 70% by weight as measured by HPLC and calculated based on dry weight of the inflorescence.

### BRIEF DESCRIPTION OF THE DRAWINGS

FIG. 1A-B. Depicts the current model biosynthetic pathway for several major cannabinoids. FIG. 1A—Geranyl pyrophosphate (GPP) and olivetolic acid (OA) are condensed by the geranyl pyrophosphate olivetolate geranyl transferase (GOT) to form cannabigerolic acid (CBGA). Alternatively, GPP and divarinic acid are condensed by GOT to form cannabigerovarinic acid (CBGVA). FIG. 1B—CBGA or CBGVA is transformed to: (1) THCA/THCVA by THCA synthase, (2) CBCA/CBCVA by CBCA synthase, or (3) CBDA/CBDVA by CBDA synthase.

FIG. 2. Depicts a sample questionnaire used for volunteer trials of Specialty Cannabis and cannabinoid compositions of the present disclosure. This questionnaire will be provided to volunteers with each cannabis blend sample or cannabinoid composition to measure the effects of the sample or cannabinoid composition when administered.

FIG. 3. Depicts the breeding scheme for the ‘GB.OJ’ high CBG cannabis line. Lines GRN01 and BLK03 were crossed to produce F1 line GN1.B3.24. Lines ORA03 and JAD21 were crossed to produce F1 line O3.J21.52. Lines GN1.B3.24 and O3.J21.52 were crossed to produce GB.OJ.28 and other high CBG progeny making up the ‘GB.OJ’ high CBG line. The CBG max content at each breeding step is provided in parenthesis under each named plant as a weight percentage based on the dry weight of the inflorescence. This breeding scheme increased the total CBG max content more than three-fold over the highest CBG max producing parental line (see Table 5).

FIG. 4. Depicts the breeding scheme for the ‘R2.R3’ high CBG cannabis line. Lines RED08 and SLV09 were crossed to produce F1 sibling lines R8.S9.02 and R8.S9.03. The R8.S9.02 and R8.S9.03 sibling lines were crossed to produce R2.R3.47, R2.R3.51, and other high CBG progeny making up the ‘R2.R3’ high CBG line. Plant R2.R3.47 was further crossed with PP.R3.08 to produce R2.R3.47-PP.R3.08 to bulk seed for NCIMB deposit 43261. Plant R2.R3.51 was also crossed with PP.R3.08 to bulk seed for NCIMB deposit 43264. Deposits are indicated by boxes with dashed lines. The CBG max content at each breeding step is provided in parenthesis under each named plant as a weight percentage based on the dry weight of the inflorescence. This breeding scheme increased the total CBG content more than five-fold over the highest CBG producing parental line (see Table 6).

FIG. 5. Depicts the breeding scheme for the ‘G2.R3’ high CBG cannabis line. Lines GLD02 and R8.S9.03 were crossed to produce G2.R3.25 and other high CBG progeny making up the ‘G2.R3’ high CBG line. Plant G2.R3.25 was further crossed with PP.R3.08 to produce G2.R3.25-PP.R3.08 to bulk seed for NCIMB deposit 43257. The CBG max content at each breeding step is provided in parenthesis under each named plant as a weight percentage based on the dry weight of the inflorescence. This breeding scheme increased the total CBG content more than seven-fold over the highest CBG producing parental line (see Table 7).

FIG. 6. Depicts the breeding scheme for the ‘PP.R3’ high CBG cannabis line. Lines PUR01 and CBD05 were crossed to produce F1 sibling lines P1.C5.10 and P1.C5.04. The P1.C5.10 and P1.C5.04 sibling lines were crossed to produce F2 line PP3. Lines RED08 and SLV09 were crossed to produce F1 line R8.S9.03. The PP03 and R8.S9.03 lines were then crossed to produce PP.R3.08 and other high CBG progeny making up the ‘PP.R3’ high CBG line. Plant PP.R3.08 was selfed to produce PP.R3.08-PP.R3.08 to bulk seed for NCIMB deposit 43263. The CBG max content at each breeding step is provided in parenthesis under each named plant as a weight percentage based on the dry weight of the inflorescence. This breeding scheme produced a (B_{T}/B_{D}) plant with eight times higher total CBG content than the original CBD5 (B_{T}/B_{D}) parental line (see Table 8).

FIG. 7. Depicts the breeding scheme for the ‘P4.R3’ high CBG cannabis line. Lines SLV08 and CBD05 were crossed to produce F1 sibling lines P08 and P01. The P08 and P01 sibling lines were crossed to produce F2 line P8.P.04. Lines RED08 and SLV09 were crossed to produce F1 line R8.S9.03. The P8.P1.04 and R8.S9.03 lines were then crossed to produce P4.R3.43 and other high CBG progeny making up the ‘P4.R3’ high CBG line. Plant P4.R3.43 was further crossed with PP.R3.08 to produce P4.R3.43-PP.R3.08 to bulk seed for NCIMB deposit 43261. The CBG max content at each breeding step is provided in parenthesis under each named plant as a weight percentage based on the dry weight of the inflorescence. This breeding scheme produced a (B_{T}/B_{D}) plant with seven times higher total CBG content than the original CBD05 (B_{T}/B_{D}) parental line (see Table 9).

### DETAILED DESCRIPTION OF THE INVENTION

All publications, patents and patent applications, including any drawings and appendices, are herein incorporated by reference to the same extent as if each individual publication or patent application was specifically and individually indicated to be incorporated by reference.

The following description includes information that may be useful in understanding the present invention. It is not an admission that any of the information provided herein is prior art or relevant to the presently claimed inventions, or that any publication specifically or implicitly referenced is prior art.

### Definition

As used herein, the verb “comprise” is used in this description and in the claims and its conjugations are used in its non-limiting sense to mean that items following the word are included, but items not specifically mentioned are not excluded.

As used herein, the term “about” refers to plus or minus 10% of the referenced number. For example, reference to an absolute content of a particular terpene of “about 1%” means that that terpene can be present at any amount ranging from 0.9% to 1.1% content by weight.

The invention provides cannabis plants. As used herein, the term “plant” refers to plants in the genus of Cannabis and plants derived thereof. Such as cannabis plants produced via asexual reproduction, tissue culture, and via seed production.

The invention provides plant parts. As used herein, the term “plant part” refers to any part of a plant including but not limited to the embryo, shoot, root, stem, seed, stipule, leaf, petal, flower, inflorescence, bud, ovule, bract, trichome, branch, petiole, internode, bark, pubescence, tiller, rhizome, frond, blade, ovule, pollen, stamen, and the like. The two main parts of plants grown in some sort of media, such as soil or vermiculite, are often referred to as the “above-ground” part, also often referred to as the “shoots”, and the “below-ground” part, also often referred to as the “roots”. Plant parts may also include certain extracts such as kief or hash, which includes cannabis trichomes or glands. In some embodiments, plant part should also be interpreted as referring to individual cells derived from the plant.

As used herein, the term “plant cell” refers to any totipotent plant cell from a cannabis plant. Plant cells of the present disclosure include cells from a cannabis plant shoot, root, stem, seed, stipule, leaf, petal, inflorescence, bud, ovule, bract, trichome, petiole, internode. In some embodiments, the disclosed plant cell is from a cannabis trichome.

As used herein, the term dominant refers to a terpene that is the most abundant in the Terpene Profile either in absolute content as a percentage by dry weight, or in relative content as a percentage of the Terpene Profile.

The term “a” or “an” refers to one or more of that entity; for example, “a gene” refers to one or more genes or at least one gene. As such, the terms “a” (or “an”), “one or more” and “at least one” are used interchangeably herein. In addition, reference to “an element” by the indefinite article “a” or “an” does not exclude the possibility that more than one of the elements is present, unless the context clearly requires that there is one and only one of the elements. Thus, the term “a plant” may refer to more than one plant.

As used herein, a “landrace” refers to a local variety of a domesticated plant species that has developed largely by natural processes, by adaptation to the natural and cultural environment in which it lives. The development of a landrace may also involve some selection by humans but it differs from a formal breed that has been selectively bred deliberately to conform to a particular formal, purebred standard of traits.

The International Code of Zoological Nomenclature defines rank, in the nomenclatural sense, as the level, for nomenclatural purposes, of a taxon in a taxonomic hierarchy (e.g., all families are for nomenclatural purposes at the same rank, which lies between superfamily and subfamily). While somewhat arbitrary, there are seven main ranks defined by the international nomenclature codes: kingdom, phylum/division, class, order, family, genus, and species. Further taxonomic hierarchies used in this invention are described below.

The invention provides plant cultivars. As used herein, the term “cultivar” means a group of similar plants that by structural features and performance (i.e., morphological and physiological characteristics) can be identified from other varieties within the same species. Furthermore, the term “cultivar” variously refers to a variety, strain or race of plant that has been produced by horticultural or agronomic techniques and is not normally found in wild populations. The terms cultivar, variety, strain and race are often used interchangeably by plant breeders, agronomists and farmers.

The term “variety” as used herein has identical meaning to the corresponding definition in the International Convention for the Protection of New Varieties of Plants (UPOV treaty), of Dec. 2, 1961, as Revised at Geneva on Nov. 10, 1972, on Oct. 23, 1978, and on Mar. 19, 1991. Thus, “variety” means a plant grouping within a single botanical taxon of the lowest known rank, which grouping, irrespective of whether the conditions for the grant of a breeder's right are fully met, can be i) defined by the expression of the characteristics resulting from a given genotype or combination of genotypes, ii) distinguished from any other plant grouping by the expression of at least one of the said characteristics and iii) considered as a unit with regard to its suitability for being propagated unchanged.

The invention provides methods for obtaining plant lines. As used herein, the term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (T0) plant regenerated from material of that line; (b) has a pedigree comprised of a T0 plant of that line; or (c) is genetically very similar due to common ancestry (e.g., via inbreeding or selfing). In this context, the term “pedigree” denotes the lineage of a plant, e.g. in terms of the sexual crosses affected such that a gene or a combination of genes, in heterozygous (hemizygous) or homozygous condition, imparts a desired trait to the plant.

As used herein, the term “inbreeding” refers to the production of offspring via the mating between relatives. The plants resulting from the inbreeding process are referred to herein as “inbred plants” or “inbreds.”

The term LOQ as used herein refers to the limit of quantitation for Gas Chromatography (GC) and High Performance Liquid Chromatography (HPLC) measurements.

The term secondary metabolites as used herein refers to organic compounds that are not directly involved in the normal growth, development, or reproduction of an organism. In other words, loss of secondary metabolites does not result in immediate death of said organism.

The term single allele converted plant as used herein refers to those plants that are developed by a plant breeding technique called backcrossing wherein essentially all of the desired morphological and physiological characteristics of an inbred are recovered in addition to the single allele transferred into the inbred via the backcrossing technique.

The invention provides samples. As used herein, the term “sample” includes a sample from a plant, a plant part, a plant cell, an extract or a composition, or from a transmission vector, or a soil, water or air sample.

The invention provides offspring. As used herein, the term “offspring” refers to any plant resulting as progeny from a vegetative or sexual reproduction from one or more parent plants or descendants thereof. For instance, an offspring plant may be obtained by cloning or selfing of a parent plant or by crossing two parent plants and include selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait), while an F2 may be (and usually is) an offspring resulting from self-pollination of said F1 hybrids.

The invention provides methods for crossing a first plant with a second plant. As used herein, the term “cross”, “crossing”, “cross pollination” or “cross-breeding” refer to the process by which the pollen of one flower on one plant is applied (artificially or naturally) to the ovule (stigma) of a flower on another plant. Backcrossing is a process in which a breeder repeatedly crosses hybrid progeny, for example a first generation hybrid (F1), back to one of the parents of the hybrid progeny. Backcrossing can be used to introduce one or more single locus conversions from one genetic background into another.

In some embodiments, the present invention provides methods for obtaining plant genotypes comprising recombinant genes. As used herein, the term “genotype” refers to the genetic makeup of an individual cell, cell culture, tissue, organism (e.g., a plant), or group of organisms.

In some embodiments, the present invention provides homozygotes. As used herein, the term “homozygote” refers to an individual cell or plant having the same alleles at one or more loci.

In some embodiments, the present invention provides homozygous plants. As used herein, the term “homozygous” refers to the presence of identical alleles at one or more loci in homologous chromosomal segments.

In some embodiments, the present invention provides hemizygotes. As used herein, the term “hemizygotes” or “hemizygous” refers to a cell, tissue, organism or plant in which a gene is present only once in a genotype, as a gene in a haploid cell or organism, a sex-linked gene in the heterogametic sex, or a gene in a segment of chromosome in a diploid cell or organism where its partner segment has been deleted.

In some embodiments, the present invention provides heterozygotes. As used herein, the terms “heterozygote” and “heterozygous” refer to a diploid or polyploid individual cell or plant having different alleles (forms of a given gene) present at least at one locus. In some embodiments, the cell or organism is heterozygous for the gene of interest that is under control of the synthetic regulatory element.

The invention provides self-pollination populations. As used herein, the term “self-crossing”, “self-pollinated” or “self-pollination” means the pollen of one flower on one plant is applied (artificially or naturally) to the ovule (stigma) of the same or a different flower on the same plant.

The invention provides ovules and pollens of plants. As used herein when discussing plants, the term “ovule” refers to the female gametophyte, whereas the term “pollen” means the male gametophyte.

The invention provides methods for obtaining plants comprising recombinant genes through transformation. As used herein, the term “transformation” refers to the transfer of nucleic acid (i.e., a nucleotide polymer) into a cell. As used herein, the term “genetic transformation” refers to the transfer and incorporation of DNA, especially recombinant DNA, into a cell.

The invention provides transformants comprising recombinant genes. As used herein, the term “transformant” refers to a cell, tissue or organism that has undergone transformation. The original transformant is designated as “T0” or “T0.” Selfing the T0 produces a first transformed generation designated as “F1” or “T1.”

As used herein, the term “cannabinoid profile” refers to the detectable cannabinoids present in a sample, such as in cannabis inflorescence material, or a composition. Thus, references to plants with novel or diverse cannabinoid profiles in this document refers plants with novel combinations or levels of cannabinoids within a single sample. The level at which cannabinoids can be detected will vary slightly depending on the techniques used, and the cannabinoid being tested.

As used herein, the term “primary cannabinoids” refers to CBGA and/or CBGVA and their decarboxylated variants, as the first cannabinoids in the model for the cannabinoid biosynthetic pathway. In contrast, the term “secondary cannabinoids” refers to any naturally produced cannabinoid that is derived from CBGA or CBGVA. Secondary cannabinoids include, but are not limited to: THC, CBD, CBC, THCV, CBDV, CBCV, and their acidic variants. In some embodiments, the terms “Non-CBG cannabinoids” or “NGCs” are used interchangeably with the term “secondary cannabinoids.”

As used herein, the term “primary cannabinoid content” refers to the additive content of the primary cannabinoids, calculated based on dry weight of the inflorescence, or the composition comprising the primary cannabinoid. The term “primary cannabinoid max content” refers to the additive content of the potential decarboxylated primary cannabinoids (as converted by formulas provided in this disclosure). This term is meant to indicate the quantity of primary cannabinoid content that would be present if all the primary cannabinoids were decarboxylated. Unless indicated otherwise, the terms “primary cannabinoid content” and “primary cannabinoid max content” are used interchangeably.

As used herein, the term “secondary cannabinoid content” refers to the additive content of the secondary cannabinoids, calculated based on dry weight of the inflorescence, or the composition comprising the secondary cannabinoid. The term “secondary cannabinoid max content” refers to the additive content of the potential decarboxylated secondary cannabinoids (as converted by formulas provided in this disclosure). This term is meant to indicate the quantity of secondary cannabinoid content that would be present if all the secondary cannabinoids were decarboxylated. Unless indicated otherwise, the terms “secondary cannabinoid content” and “secondary cannabinoid max content” are used interchangeably.

As used herein, the term “high CBG content” refers to non-trace (i.e., > or =1.0%) of CBG max content. In some embodiments, high CBG plants and compositions of the present disclosure will have significantly higher than 1.0% CBG max content.

As used herein, the term “high primary cannabinoid” refers to non-trace (i.e., > or =1.0%) of the additive content CBG max and CBGv max. In some embodiments, high primary cannabinoid plants and compositions of the present disclosure will have significantly higher than 1.0% additive CBG max and CBGVmax content. In some embodiments, the terms “high primary cannabinoid” and “high CBG/CBGV” content are interchangeably used.

As used herein, the term “propyl cannabinoids” refers to cannabinoids with a propyl side chain in place of the normal pentyl side chain. In some embodiments, propyl cannabinoids comprise THCV, CBDV, and CBCV and their acidic variants. Propyl cannabinoids in the context of this disclosure exclude CBGV, which is instead referred to in connection with CBG as one of the “primary cannabinoids.”

As used herein, the term “propyl cannabinoid content” refers to the additive content of the propyl cannabinoids, as measured by dry weight of the inflorescence, or the composition comprising the propyl cannabinoid. The term “propyl cannabinoid max content” refers to the additive content of the potential decarboxylated propyl cannabinoids (as converted by formulas provided in this disclosure). This term is meant to indicate the quantity of propyl cannabinoid content that would be present if all the propyl cannabinoids were decarboxylated. Unless indicated otherwise, the terms “propyl cannabinoid content” and “propyl cannabinoid max content” are used interchangeably.

In some embodiments, the present disclosure refers to BT, BD, or B0 alleles. As used herein, the term “BT allele” or “BT allele” refers to a gene coding for a THCA synthase enzyme.

As used herein, the term “BD allele” or “BD allele” refers to a gene coding for a CBDA synthase enzyme. As used herein, the term “B0 allele” or “B₀ allele” refers to a gene coding for a null THCA or CBDA synthase enzyme. Thus, a BT allele containing cannabis plant would be expected to accumulate THCA/THCVA, and a BD allele containing cannabis plant would be expected to accumulate CBDA/CBDVA. A plant cannabis plant comprising only B0 alleles for a CBDA synthase enzyme would not be expected to accumulate CBGA/CBGVA primary cannabinoid, through trace quantities of other cannabinoids may accumulate.

BT, BD, and B0 alleles are detectable through direct sequencing (Onofri et al., 2015 “Sequence heterogeneity of Cannabidiolic- and tetrahydrocannabinolic acid-synthase in Cannabis sativa L. and its relationship with chemical phenotype” Phytochemistry Vol 116 pgs 57-68). Persons having skill in the art can also determine the presence of a BT, BD, or homozygous B0 alleles by studying the cannabinoid profile of the plant. BT alleles result in the accumulation of THCA and/or THCVA, while BD alleles result in the accumulation of CBDA and/org CBDVA. Homozygous B0 alleles result in plants with only small amounts of THCA and CBDA, with CBDA typically reaching slightly higher levels than THCA. Thus, for the purposes of this application, genotype at the B allele can be assessed by analyzing the cannabinoid profile of cannabis tissue.

As used herein, the term “functional B_{T} allele” or “functional B_{D} allele” refers to an allele that results in the cannabis plant accumulating greater than 1.5% THCmax/THCVmax or greater than 2.0% CBDmax/CBDVmax, respectively. Cannabinoid accumulation below this level is typically attributed to residual activity of otherwise “null” alleles.

Unless otherwise noted, references to cannabinoids in a plant, plant part, extract, or composition of the present disclosure should be understood as references to both the acidic and decarboxylated versions of the compound (e.g., THCmax as determined by the conversion guidelines described in this document, and understood by those skilled in the art). For example, references to high THC contents of a cannabis plant in this disclosure should be understood as referencing to the combined THC and THCA content (THCmax).

The terms THCmax and THC max are interchangeably used in this document. This is true for all other cannabinoids discussed in this document.

As used herein, the term “winterizing” or “winterization” refers to the process by which plant lipids and waxes are removed from a cannabis extract. Persons have skill in the art will immediately recognize how to winterize an extract. Briefly, winterization is the dissolving the cannabis extract into a polar solvent (most commonly ethanol) at sub-zero temperatures. Doing so separates the waxes and lipids from the oil, forcing them to collect at the top of the mixture for easy filtration/collection. Typically, winterization is conducted by mixing ethanol and hash oil into a container and placing it into a sub-zero freezer.

As used herein, the term “maturity,” “harvest maturity,” or “floral maturity” refers to the developmental stage at which a cannabis plant is ready for harvest. Persons having skill in the art will recognize maturity based on the plant's morphologies. Cannabis plants are considered to be at harvest maturity when fan leaves begin to yellow, and when inflorescences begin to take on a ‘frosted’ appearance, as trichomes develop on calyxes and lower portions of bracts. If bracts and inflorescent parts turn overly yellow and/or if the ‘frosted’ appearance is visible from afar, this could indicate the plant is beyond maturity. The color of trichomes can also be used to determine maturity. Trichomes from cannabis plants first look small and clear, but gradually enlarge, and progressively become ‘milkier’ and opaque with continued maturation, finally displaying a desiccated appearance and amber color. In the present disclosure, harvest maturity is defined as the time period between the enlarged clear trichome developmental stage and the opaque/milky trichome developmental stage. Amber trichomes in cannabis plants are, in some embodiments, an indication of overly mature trichomes. The present disclosure uses the terms “maturity,” “harvest maturity,” and “floral maturity” interchangeably. Unless otherwise noted, all cannabinoid and terpene values of cannabis plants discussed in this document refer to the level of those compounds present in a cannabis inflorescence at harvest maturity.

As used herein, the term “Terpene Profile” is defined as the absolute and relative values of 17 of the most expressed terpenes in the Specialty Cannabis hemp and compositions of the present disclosure: terpinolene, alpha phellandrene, beta ocimene, carene, limonene, gamma terpinene, alpha pinene, alpha terpinene, beta pinene, fenchol, camphene, alpha terpineol, alpha humulene, beta caryophyllene, linalool, caryophyllene oxide, and myrcene. A survey of the terpene profiles of several cannabis varieties has found that these terpenes express at high enough levels so as to have their own pharmacological effects and also to act in synergy with cannabinoids.

As used herein, the term “Terpene Essential Oil” or “Terpene Essential Oil Content” refers to the additive contents of all the terpenes in the Terpene Profile, represented by weight of the dry inflorescence or cannabinoid composition. In some embodiments, the terms “terpene oil content” and “terpene essential oil content” are used interchangeably.

### Cannabis

Cannabis is an annual, dioecious, flowering herb. Its leaves are typically palmately compound or digitate, with serrated leaflets. Cannabis normally has imperfect flowers, with staminate “male” and pistillate “female” flowers occurring on separate plants. It is not unusual, however, for individual plants to separately bear both male and female flowers (i.e., have monoecious plants). Although monoecious plants are often referred to as “hermaphrodites,” true hermaphrodites (which are less common in cannabis) bear staminate and pistillate structures on individual flowers, whereas monoecious plants bear male and female flowers at different locations on the same plant.

The life cycle of cannabis varies with each variety but can be generally summarized into germination, vegetative growth, and reproductive stages. Because of heavy breeding and selection by humans, most cannabis seeds have lost dormancy mechanisms and do not require any pre-treatments or winterization to induce germination (See Clarke, R C et al. “Cannabis: Evolution and Ethnobotany” University of California Press 2013). Seeds placed in viable growth conditions are expected to germinate in about 3 to 7 days. The first true leaves of a cannabis plant contain a single leaflet, with subsequent leaves developing in opposite formation, with increasing number of leaflets. Leaflets can be narrow or broad depending on the morphology of the plant grown. Cannabis plants are normally allowed to grow vegetatively for the first 4 to 8 weeks. During this period, the plant responds to increasing light with faster and faster growth. Under ideal conditions, cannabis plants can grow up to 2.5 inches a day, and are capable of reaching heights of 20 feet or more. Indoor growth pruning techniques tend to limit cannabis size through careful pruning of apical or side shoots.

Cannabis has long been used for drug and industrial purposes, including fiber (hemp), for seed and seed oils, for medicinal purposes, and as a recreational drug. Industrial hemp products are made from cannabis plants selected to produce an abundance of fiber. In some embodiments, hemp varieties of Cannabis have been bred to produce minimal levels of THC, the principal psychoactive constituent responsible for the psychoactivity associated with marijuana. “Marijuana” varieties of Cannabis on the other hand typically refer to plants that have been bred to produce high levels of THC and other secondary metabolites, including other cannabinoids and terpenes.

Hemp strains generally refer to fiber-producing cannabis plants that exhibit tall unbranched (sativa-like) morphologies. These plants have been bred to focus their energies on producing long fibrous stalks and generally only accumulate low levels of cannabinoid drug compounds, which can be used for recreational or medicinal applications. “Drug type” cannabis (“Marijuana”) strains on the other hand, refer to plants that are designed for human recreational or medicinal consumption. These plants focus their energies on producing large numbers of resinous flowers, and thus typically exhibit shorter, highly branched morphologies adapted to indoor grows.

Although marijuana cannabis strains used as a drug and industrial hemp both derive from the Cannabis family and contain trace amounts or more of the psychoactive component tetrahydrocannabinol (THC), they are distinct strains with unique phytochemical compositions and uses. Hemp typically has lower concentrations of THC and higher concentrations of cannabidiol (CBD), which decreases or eliminates the psychoactive effects of the plant. The legality of industrial hemp varies widely between countries. Some governments regulate the concentration of THC and permit only hemp that is bred with an especially low THC content.

In contrast to cannabis for medical use, varieties grown for fiber and seed typically have less than 0.3% THC and are unsuitable for producing hashish and marijuana (Sawler J et al, 2015, PLOS One. 10(8): e0133292). Present in industrial hemp, cannabidiol is a major constituent among some 560 compounds found in hemp. The major differences between the two types of plants are the appearance, and the amount of Δ9-tetrahydrocannabinol (THC) secreted in a resinous mixture by epidermal hairs called glandular trichomes, although they can also be distinguished genetically. Oilseed and fiber varieties of Cannabis approved for industrial hemp production produce only minute amounts of this psychoactive drug, not enough for any physical or psychological effects. Typically, hemp contains below 0.3% THC, while cultivars of Cannabis grown for medicinal or recreational use can contain anywhere from 2% to over 20%.

In 2014, President Obama signed the Agricultural Act of 2014, (a.k.a. the 2014 Farm Bill) which included Section 7606 allowing for universities and state departments of agriculture to begin cultivating industrial hemp for limited purposes. Specifically, the law allows universities and state departments of agriculture to grow or cultivate industrial hemp if: “(1) the industrial hemp is grown or cultivated for purposes of research conducted under an agricultural pilot program or other agricultural or academic research; and (2) the growing or cultivating of industrial hemp is allowed under the laws of the state in which such institution of higher education or state department of agriculture is located and such research occurs.” For purposes of the Farm Bill, industrial hemp is defined as Cannabis sativa L., having a THC concentration ≤0.3%.

The law also requires that the grow sites be certified by—and registered with—their state. A bipartisan group of U.S. senators introduced the Industrial Hemp Farming Act of 2015 that would allow American farmers to produce and cultivate industrial hemp. The bill would remove hemp from the controlled substances list as long as it contained no more than 0.3 percent THC. The U.S. Department of Agriculture, in consultation with the U.S. Drug Enforcement Agency (DEA) and the U.S. Food and Drug Administration, released a Statement of Principles on Industrial Hemp in the Federal Register on Aug. 12, 2016, on the applicable activities related to hemp in the 2014 Farm Bill.

Hemp is used to make a variety of commercial and industrial products including rope, clothes, food, paper, textiles, plastics, insulation and biofuel. The bast fibers can be used to make textiles that are 100% hemp, but they are commonly blended with other organic fibers such as flax, cotton or silk, to make woven fabrics for apparel and furnishings. The inner two fibers of the plant are more woody and typically have industrial applications, such as mulch, animal bedding and litter. When oxidized (often erroneously referred to as “drying”), hemp oil from the seeds becomes solid and can be used in the manufacture of oil-based paints, in creams as a moisturizing agent, for cooking, and in plastics. Hemp seeds have been used in bird feed mix as well. Also, more than 95% of hemp seed sold in the European Union was used in animal and bird feed according to the 2013 research data. Thus, the hemp seed can be used for animal and bird feed.

Cannabis is diploid, having a chromosome complement of 2n=20, although polyploid individuals have been artificially produced. The first genome sequence of Cannabis, which is estimated to be 820 Mb in size, was published in 2011 by a team of Canadian scientists (van Bakel et al, “The draft genome and transcriptome of Cannabis sativa” Genome Biology 12:R102).

All known strains of Cannabis are wind-pollinated and the fruit is an achene. Most strains of Cannabis are short day plants, with the possible exception of C. sativa subsp. sativa var. spontanea (=C. ruderalis), which is commonly described as “auto-flowering” and may be day-neutral.

Although, some cannabis varieties will flower without the need for external stimuli, most varieties have an absolute requirement for inductive photoperiods in the form of short days or long nights to induce fertile flowering. The first sign of flowering in cannabis is the appearance of undifferentiated flower primordial along the main stem of the nodes. At this stage, the sex of the plants are still not distinguishable. As the flower primordia continue to develop, female (pistillate), and male (staminate) flowers can be distinguished.

For most cannabinoid producing purposes, only female plants are desired. The presence of male flowers is considered undesirable, as pollination is known to reduce the cannabinoid yield, and potentially ruin a crop. For this reason, most cannabis is grown “sinsemilla” (seedless), through vegetative (i.e., asexual) propagation. In this way, only female plants are produced and no space is wasted on male plants. Industrial hemp plants are in some instances propagated via feminized seed. Resinous hemp is nearly always grown from feminized seeds to avoid possible pollination, which greatly reduces the cannabinoid yield of plants. Thus, in some embodiments, the plants and inflorescences of the present disclosure are seedless, sinsemilla. In some embodiments, the plants and inflorescences of the present disclosure are unpollinated.

### Cannabis Chemistry—Cannabinoids

Cannabis plants produce a unique family of terpeno-phenolic compounds called cannabinoids. Cannabinoids, terpenoids, and other compounds are secreted by glandular trichomes that occur most abundantly on the floral calyxes and bracts of female plants. As a drug it usually comes in the form of dried flower buds (marijuana), resin (hashish), or various extracts collectively known as hashish oil. There are at least 483 identifiable chemical constituents known to exist in the cannabis plant (Rudolf Brenneisen, 2007, Chemistry and Analysis of Phytocannabinoids (cannabinoids produced by cannabis) and other Cannabis Constituents, In Marijuana and the Cannabinoids, ElSohly, ed.; incorporated herein by reference) and at least 85 different cannabinoids have been isolated from the plant (El-Alfy, Abir T, et al., 2010, “Antidepressant-like effect of delta-9-tetrahydrocannabinol and other cannabinoids isolated from Cannabis sativa L”, Pharmacology Biochemistry and Behavior 95 (4): 434-42; incorporated herein by reference). The two cannabinoids usually produced in greatest abundance are cannabidiol (CBD) and/or Δ9-tetrahydrocannabinol (THC). THC is psychoactive while CBD is not. See, ElSohly, ed. (Marijuana and the Cannabinoids, Humana Press Inc., 321 papers, 2007), which is incorporated herein by reference in its entirety, for a detailed description and literature review on the cannabinoids found in marijuana.

Cannabinoids accumulate at the highest levels in the trichomes of cannabis inflorescences. However, cannabinoids have been detected in nearly all cannabis organs (see John K. Hemphill et al, “Cannabinoid Content of Individual Plant Organs From Different Geographical Strains of Cannabis Sativa L” Journal of Natural Products, Vol 43, No. 1 January-February, 1980). Applicant has similarly detected terpenes in non-inflorescence parts of cannabis plants. Thus, in some embodiments, the plant cells of the present disclosure are terpene and cannabinoid producing cells.

Cannabinoids are the most studied group of secondary metabolites in cannabis. Most exist in two forms, as acids and in neutral (decarboxylated) forms. The acid form is designated by an “A” at the end of its acronym (i.e. THCA). The phytocannabinoids are synthesized in the plant as acid forms, and while some decarboxylation does occur in the plant, it increases significantly post-harvest and the kinetics increase at high temperatures. (Sanchez and Verpoorte 2008). The biologically active forms for human consumption are the neutral forms.

As discussed above, all cannabinoids in their acid forms (those ending in “−A”) can be converted to their non-acidic forms through a process called decarboxylation. Decarboxylation is usually achieved by thorough drying of the plant material followed by heating it, often by either combustion, vaporization, or heating or baking in an oven. Cannabinoid compositions can similarly be decarboxylated by being exposed to heat.

In order to find the total amount of cannabinoids in a sample (e.g., total amount of active non-acidic cannabinoid), the total measured content of acid cannabinoid variants forms should be adjusted to account for the loss of the carboxyl group. In some embodiments, this adjustment can be made by multiplying the molar content of the acidic cannabinoid forms by the molecular weight of the corresponding decarboxylated cannabinoid. Other shorthand conversions are also available for quickly converting acidic cannabinoid content to active cannabinoid content.

For example, in some embodiments, THCA can be converted to active THC using the formula: THCA×0.877=THC. When using this approach, the maximum THC for the sample is: THCmax=(THCA×0.877)+THC. This method has been validated according to the principles of the International Conference on Harmonization. Similarly, CBDA can be converted to active CBD and the yield is determined using the yield formula: CBDA×0.877=CBD. Also, the maximum amount of CBD yielded, i.e. max CBD for the sample is: CBDmax=(CBDA×0.877)+CBD. Additionally, CBGA can be converted to active CBG by multiplying CBGA by 0.878 (CBGmax=(CBGA×0.878)+CBG). THCVA and CBDVA can be converted to THCV and CBDV, respectively by multiplying their acidic contents by 0.8668 (THCVmax=(THCVA×0.8668)+THCV; CBDVmax=(CBDVA×0.8668)+CBDV). CBGVA can be converted to CBGV by multiplying CBGVA by 0.8676 (CBGVmax=(CBGVA×0.8676)+CBGV).

Unless otherwise noted, references to cannabinoids in a plant, plant part, extract, or composition of the present disclosure includes both the acidic and decarboxylated versions of the compound (e.g., THCmax as determined by the conversion guidelines described above, and understood by those skilled in the art). References to a cannabinoid content (however it is measured) in a claim should be understood as representing theoretical maximums of decarboxylated “active” cannabinoid contents, plus converted contents of acidic versions of the same cannabinoid, unless otherwise indicated.

The cannabinoids in the Specialty Cannabis plants, plant parts, extracts and compositions of the present disclosure include, but are not limited to, Δ9-Tetrahydrocannabinol (Δ9-THC), Δ8-Tetrahydrocannabinol (Δ8-THC), Cannabichromene (CBC), Cannabicyclol (CBL), Cannabidiol (CBD), Cannabielsoin (CBE), Cannabigerol (CBG), Cannabinidiol (CBND), Cannabinol (CBN), Cannabitriol (CBT), and their propyl homologs, including, but are not limited to cannabidivarin (CBDV), Δ9-Tetrahydrocannabivarin (THCV), cannabichromevarin (CBCV), and cannabigerovarin (CBGV), and their acidic variants. See Holley et al. (Constituents of Cannabis sativa L. XI Cannabidiol and cannabichromene in samples of known geographical origin, J. Pharm. Sci. 64:892-894, 1975) and De Zeeuw et al. (Cannabinoids with a propyl side chain in Cannabis, Occurrence and chromatographic behavior, Science 175:778-779), each of which is herein incorporated by reference in its entirety for all purposes.

Non-CBG cannabinoids can be collectively referred to as “NGCs”, wherein a NGC can be one or more of THC, THCV, CBD, CBDV, CBC, CBCV, CBN, and Δ 8THC (aka D8THC) cannabinoids, and their acidic variants. Notably, NGCs do not include CBGV, which is considered for the purposes of this disclosure to be a CBG cannabinoid. Thus, reference to 5% NGC content will be understood as referring to a 5% content of the additive content of THC, THCV, CBD, CBDV, CBC, CBCV, CBN, and D8THC cannabinoids, and their acidic variants.

Brief descriptions and chemical structures for several of the major cannabinoids are provided below.

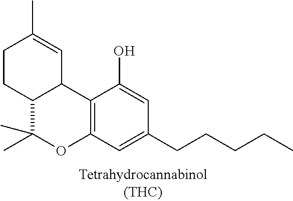

Known as delta-9-tetrahydrocannabinol (Δ9-THC), THC is the principal psychoactive constituent (or cannabinoid) of the cannabis plant. The initially synthesized and accumulated form in plant is THC acid (THCA).

THC has mild to moderate analgesic effects, and cannabis can be used to treat pain by altering transmitter release on dorsal root ganglion of the spinal cord and in the periaqueductal gray. Other effects include relaxation, alteration of visual, auditory, and olfactory senses, fatigue, and appetite stimulation. THC has marked antiemetic properties, and may also reduce aggression in certain subjects (Hoaken (2003). “Drugs of abuse and the elicitation of human aggressive behavior”. Addictive Behaviors 28: 1533-1554).

The pharmacological actions of THC result from its partial agonist activity at the cannabinoid receptor CB1, located mainly in the central nervous system, and the CB2 receptor, mainly expressed in cells of the immune system (Pertwee, 2006, “The pharmacology of cannabinoid receptors and their ligands: An overview”. International Journal of Obesity 30: S13-S18.) The psychoactive effects of THC are primarily mediated by its activation of CB1G-protein coupled receptors, which result in a decrease in the concentration of the second messenger molecule cAMP through inhibition of adenylate cyclase (Elphick et al., 2001, “The neurobiology and evolution of cannabinoid signaling”. Philosophical Transactions of the Royal Society B: Biological Sciences 356 (1407): 381-408.) It is also suggested that THC has an anticholinesterase action, which may implicate it as a potential treatment for Alzheimer's and Myasthenia (Eubanks et al., 2006, “A Molecular Link Between the Active Component of Marijuana and Alzheimer's Disease Pathology”. Molecular Pharmaceutics 3 (6): 773-7.)

In the cannabis plant, THC occurs mainly as tetrahydrocannabinolic acid (THCA, 2-COOH-THC). Geranyl pyrophosphate and olivetolic acid react, catalyzed by an enzyme to produce cannabigerolic acid, which is cyclized by the enzyme THC acid synthase to give THCA. Over time, or when heated, THCA is decarboxylated producing THC. The pathway for THCA biosynthesis is similar to that which produces the bitter acid humulone in hops. See Fellermeier et al., (1998, “Prenylation of olivetolate by a hemp transferase yields cannabigerolic acid, the precursor of tetrahydrocannabinol”. FEBS Letters 427 (2): 283-5); de Meijer et al. I, II, II, and IV (I: 2003, Genetics, 163:335-346; II: 2005, Euphytica, 145:189-198; II: 2009, Euphytica, 165:293-311; and IV: 2009, Euphytica, 168:95-112.)

Non-limiting examples of THC variants include:

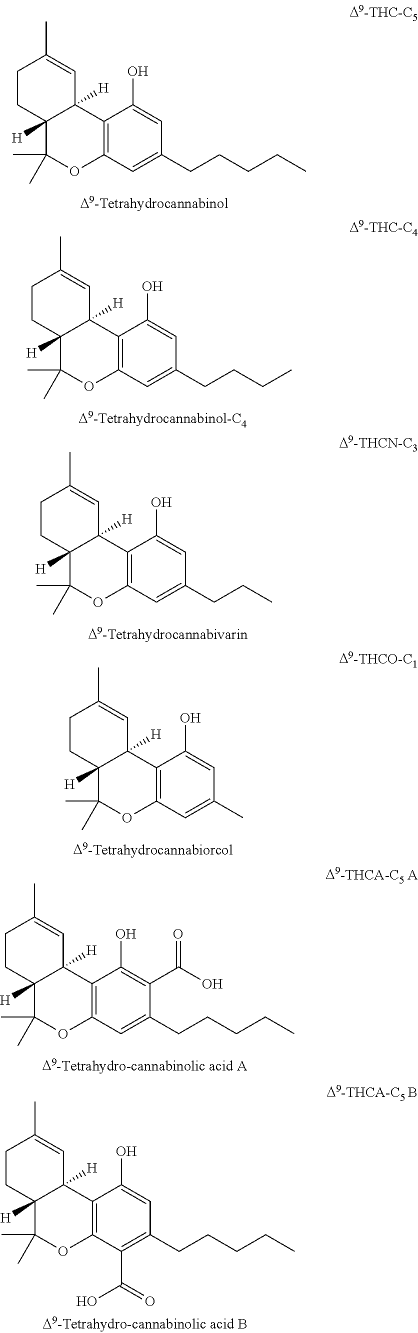

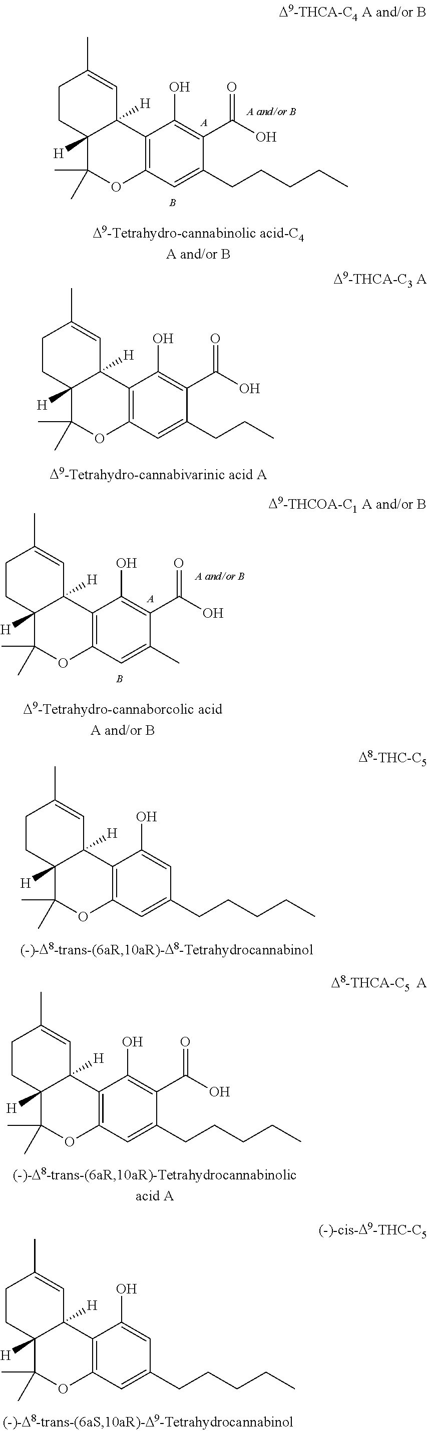

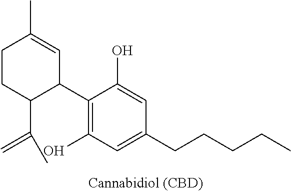 [SMILES: C=C(C)C1CCC(C)=CC1c1c(O)cc(CCCCC)cc1O]

CBD is a cannabinoid found in cannabis. Cannabidiol has displayed sedative effects in animal tests (Pickens, 1981, “Sedative activity of cannabis in relation to its delta'-trans-tetrahydrocannabinol and cannabidiol content”. Br. J. Pharmacol. 72 (4): 649-56). Some research, however, indicates that CBD can increase alertness, and attenuate the memory-impairing effect of THC. (Nicholson et al., June 2004, “Effect of Delta-9-tetrahydrocannabinol and cannabidiol on nocturnal sleep and early-morning behavior in young adults” J Clin Psychopharmacol 24 (3): 305-13; Morgan et al., 2010, “Impact of cannabidiol on the acute memory and psychotomimetic effects of smoked cannabis: naturalistic study, The British Journal of Psychiatry, 197:258-290). It may decrease the rate of THC clearance from the body, perhaps by interfering with the metabolism of THC in the liver. Medically, it has been shown to relieve convulsion, inflammation, anxiety, and nausea, as well as inhibit cancer cell growth (Mechoulam, et al., 2007, “Cannabidiol—recent advances”. Chemistry & Biodiversity 4 (8): 1678-1692.) Recent studies have shown cannabidiol to be as effective as atypical antipsychotics in treating schizophrenia (Zuardi et al., 2006, “Cannabidiol, a Cannabis sativa constituent, as an antipsychotic drug” Braz. J. Med. Biol. Res. 39 (4): 421-429.). Studies have also shown that it may relieve symptoms of dystonia (Consroe, 1986, “Open label evaluation of cannabidiol in dystonic movement disorders”. The International journal of neuroscience 30 (4): 277-282). CBD reduces growth of aggressive human breast cancer cells in vitro and reduces their invasiveness (McAllister et al., 2007, “Cannabidiol as a novel inhibitor of Id-1 gene expression in aggressive breast cancer cells”. Mol. Cancer Ther. 6 (11): 2921-7.)

Cannabis produces CBD-carboxylic acid through the same metabolic pathway as THC, until the last step, where CBDA synthase performs catalysis instead of THCA synthase. See Marks et al. (2009, “Identification of candidate genes affecting Δ9-tetrahydrocannabinol biosynthesis in Cannabis sativa”. Journal of Experimental Botany 60 (13): 3715-3726.) and Meijer et al. I, II, III, and IV. Non-limiting examples of CBD variants include:

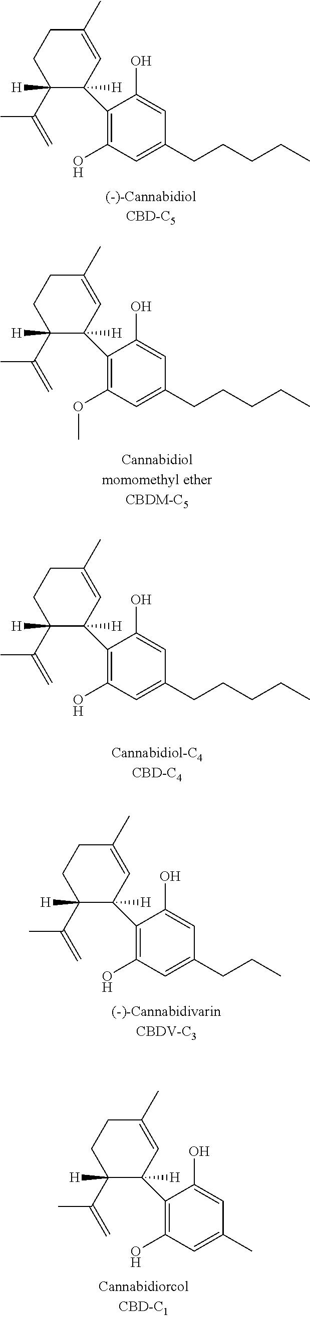

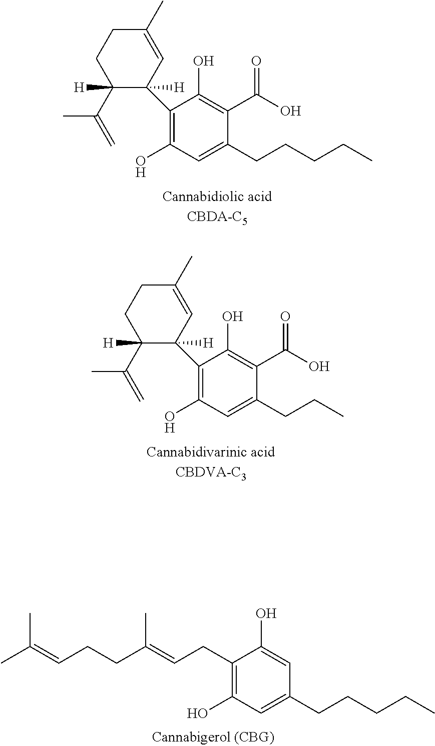

CBG is a non-psychoactive cannabinoid found in the Cannabis genus of plants. Cannabigerol is found in higher concentrations in hemp rather than in varieties of Cannabis cultivated for high THC content and their corresponding psychoactive properties. Cannabigerol has been found to act as a high affinity α2-adrenergic receptor agonist, moderate affinity 5-HT1A receptor antagonist, and low affinity CB1 receptor antagonist. It also binds to the CB2 receptor. Cannabigerol has been shown to relieve intraocular pressure, which may be of benefit in the treatment of glaucoma (Craig et al. 1984, “Intraocular pressure, ocular toxicity and neurotoxicity after administration of cannabinol or cannabigerol” Experimental eye research 39 (3):251-259). Cannabigerol has also been shown to reduce depression in animal models (U.S. patent application Ser. No. 11/760,364). In particular CBG has been shown to have significant potential applications in the treatment of glaucoma, depression, Huntington's disease, MRSA, cachexia, and cancer (Craig et al. 1984, “Intraocular pressure, ocular toxicity and neurotoxicity after administration of cannabinol or cannabigerol” Experimental eye research 39 (3):251-259; U.S. Pat. No. 8,481,085; Valdeolivas et al. 2015 “Neuroprotective properties of cannabigerol in Huntington's disease; studies in R6/2 mice and 30nitropropionate-lesioned mice.” Neurotherapeutics January 12(1):185-99; Appendino G et al., 2008 “Antibacterial cannabinoids from Cannabis sativa: a structure-activity study” J. Nat Prod. August:71(8):1427-30; Borrelli F et al. 2013 “Beneficial effect of the non-psychotropic plant cannabinoid cannabigerol on experimental inflammatory bowel disease” Biochem Pharmacol May 1:85(9):1306-16; Borrelli F. et al. 2014 “Colon carcinogenesis is inhibited by the TRPM8 antagonist cannabigerol, a Cannabis-derived non-psychotropic cannabinoid” Carcinogenesis December: 35(12):2787-97) Non-limiting examples of CBG variants include:

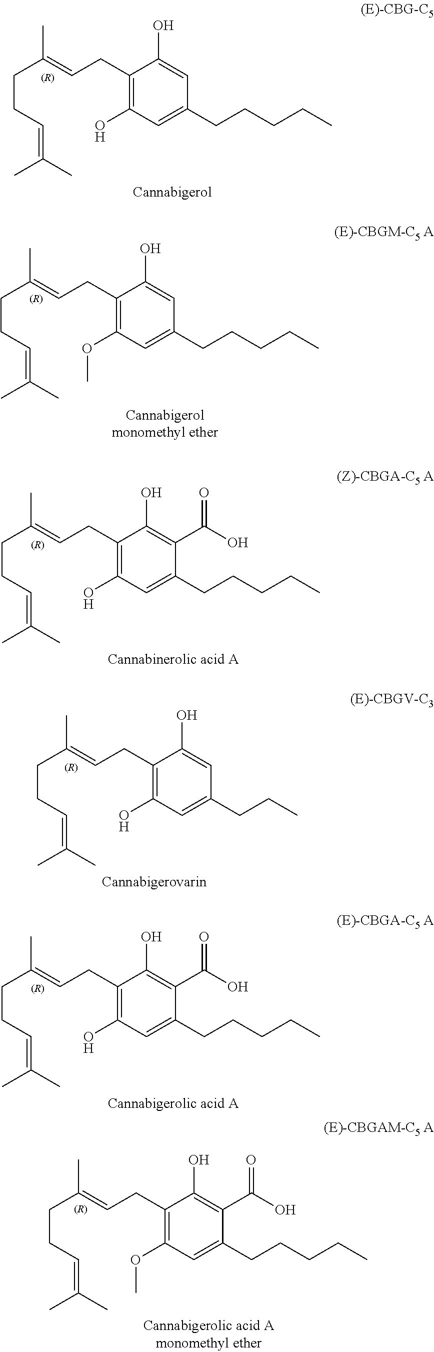

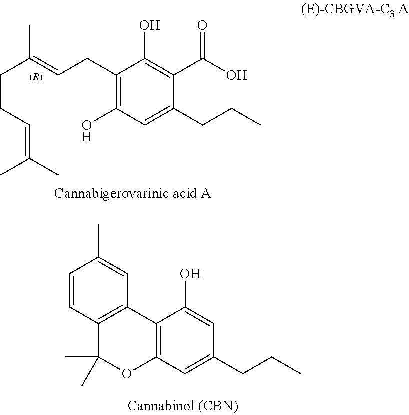

CBN is a mildly to non-psychoactive substance cannabinoid found in Cannabis sativa and Cannabis indica/afghanica. It is also a metabolite of tetrahydrocannabinol (THC). CBN acts as a weak agonist of the CB1 and CB2 receptors, with lower affinity in comparison to THC. Non-limiting examples of CBN variants include

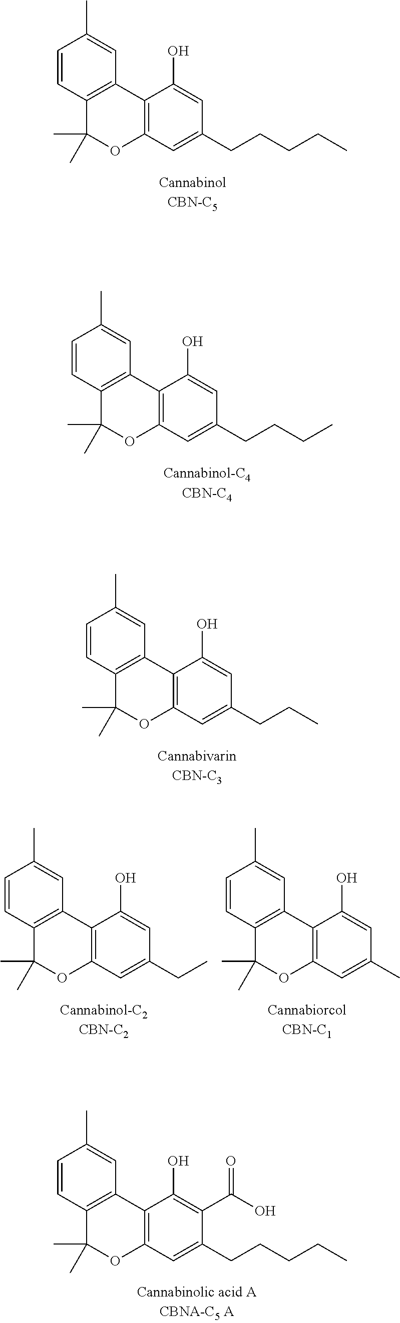

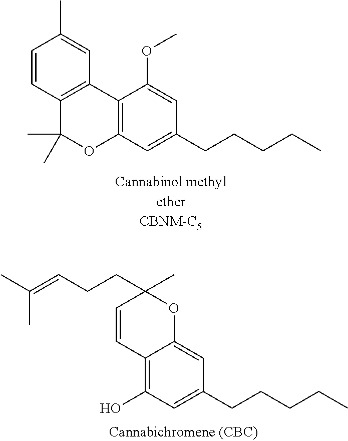

CBC bears structural similarity to the other natural cannabinoids, including tetrahydrocannabinol, tetrahydrocannabivarin, cannabidiol, and cannabinol, among others. Evidence has suggested that it may play a role in the anti-inflammatory and anti-viral effects of cannabis, and may contribute to the overall analgesic effects of cannabis. Non-limiting examples of CBC variants include:

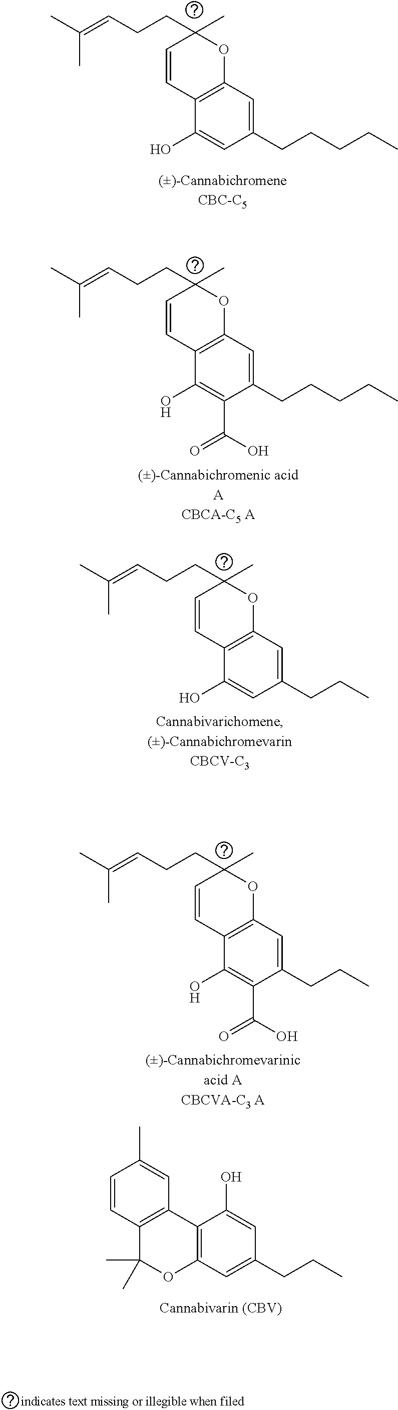

Cannabivarin, also known as cannabivarol or CBV, is a non-psychoactive cannabinoid found in minor amounts in the hemp plant Cannabis sativa. It is an analog of cannabinol (CBN) with the side chain shortened by two methylene bridges (—CH2—). CBV is an oxidation product of tetrahydrocannabivarin (THCV, THV).

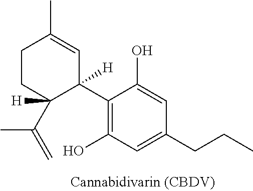 [SMILES: C=C(C)[C@@H]1CCC(C)=C[C@H]1c1c(O)cc(CCC)cc1O]

CBDV is a non-psychoactive cannabinoid found in Cannabis. It is a homolog of cannabidiol (CBD), with the side-chain shortened by two methylene bridges (CH2 units). Cannabidivarin has been found reduce the number and severity of seizures in animal models (U.S. patent application Ser. No. 13/075,873). Plants with relatively high levels of CBDV have been reported in feral populations of C. indica (=C. sativa ssp. indica var. kafiristanica) from northwest India, and in hashish from Nepal.

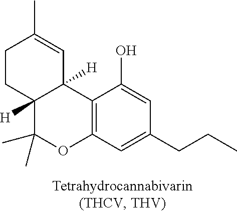 [SMILES: CCCc1cc(O)c2c(c1)OC(C)(C)[C@@H]1CCC(C)=C[C@@H]21]

THCV, or THV is a homologue of tetrahydrocannabinol (THC) having a propyl (3-carbon) side chain. This terpeno-phenolic compound is found naturally in Cannabis, sometimes in significant amounts. Plants with elevated levels of propyl cannabinoids (including THCV) have been found in populations of Cannabis sativa L. ssp. indica (=Cannabis indica Lam.) from China, India, Nepal, Thailand, Afghanistan, and Pakistan, as well as southern and western Africa. THCV has been shown to be a CB1 receptor antagonist, i.e. it blocks the effects of THC. Tetrahydrocannabinol has been shown to increase metabolism, help weight loss and lower cholesterol in animal models (U.S. patent application Ser. No. 11/667,860)

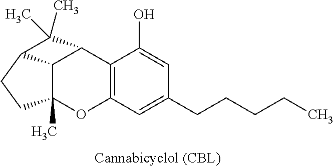

Cannabicyclol (CBL) is a non-psychotomimetic cannabinoid found in the Cannabis species. CBL is a degradative product like cannabinol. Light converts cannabichromene to CBL. Non-limiting examples of CBL variants include:

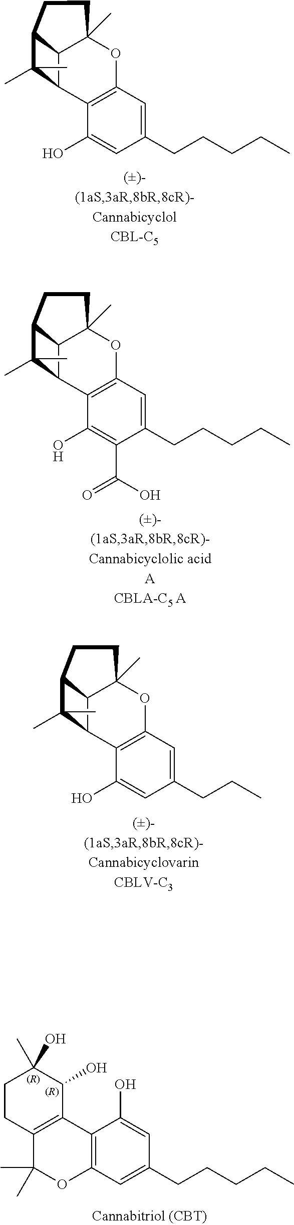

Non-limiting examples of CBT variants include:

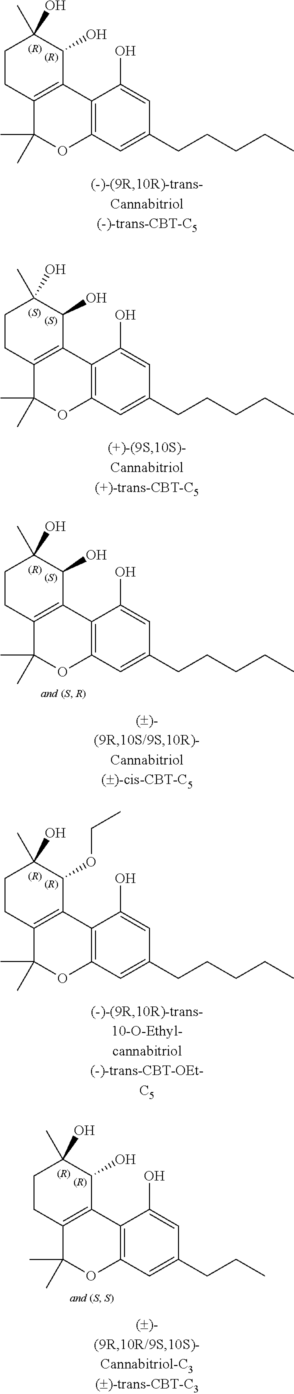

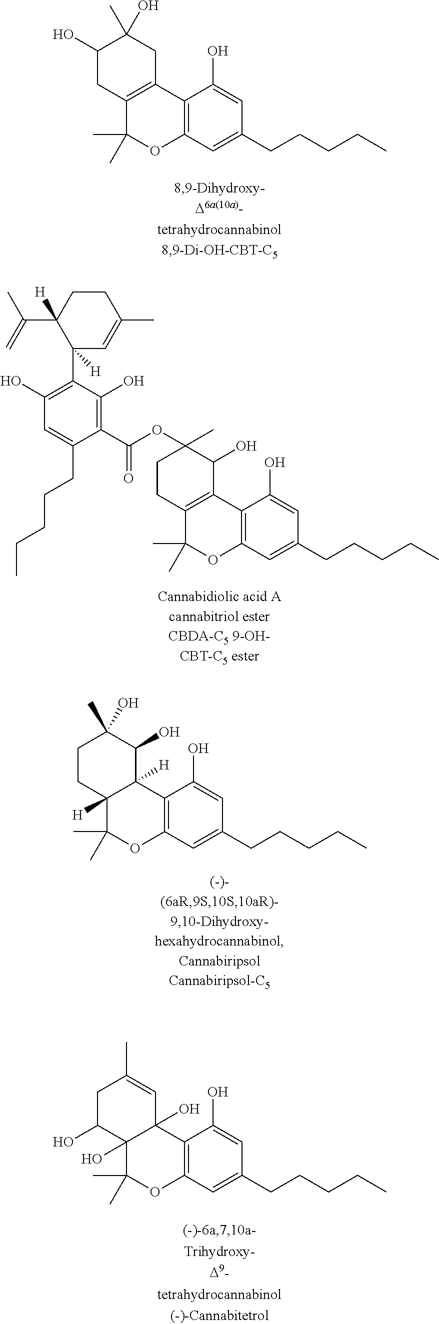

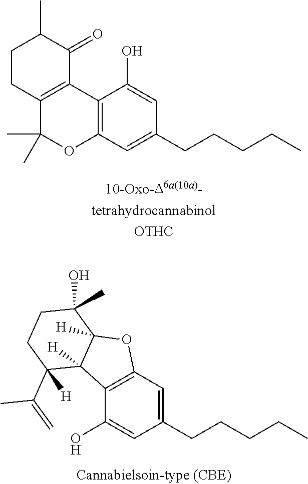

Non-limiting examples of CBE variants include:

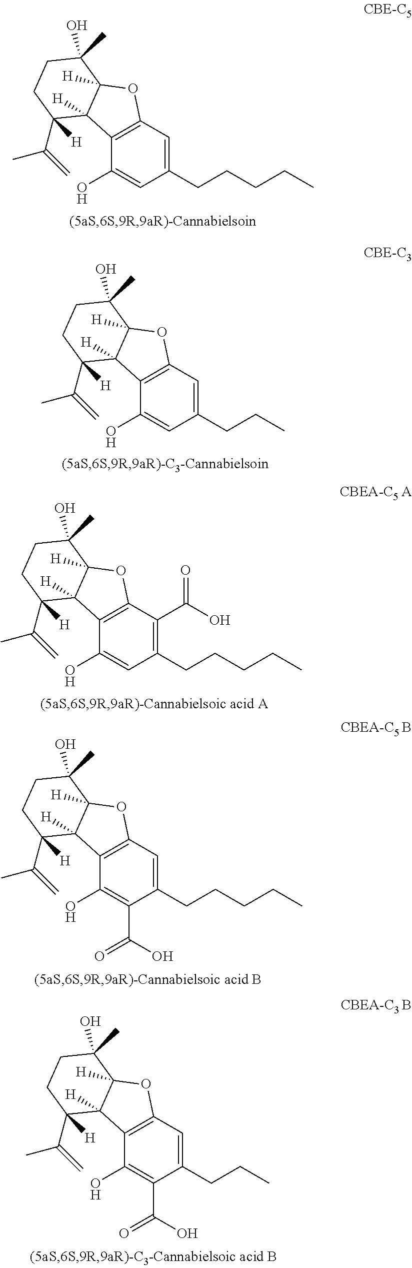

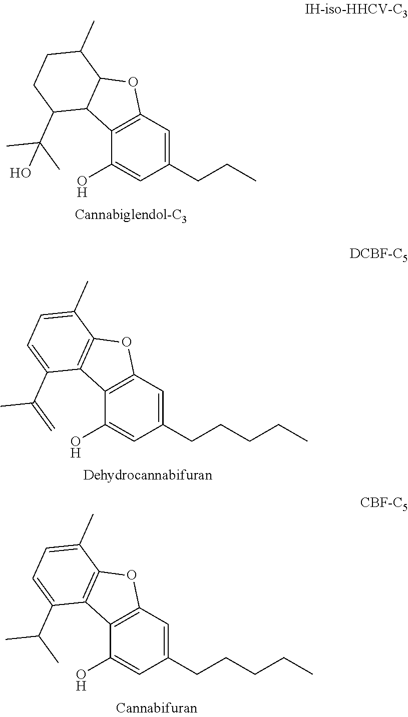

More details of cannabinoids synthesis and the properties and uses of these cannabinoids are described in Russo (2011, Taming THC: potential cannabis synergy and phytocannabinoid-terpenoid entourage effects, British Journal of Pharmacology, 163:1344-1364), Russo et al. (2006, A tale of two cannabinoids: the therapeutic rationale for combining tetrahydrocannabinol and cannabidiol, Medical Hypothesis, 2006, 66:234-246), Celia et al. (Impact of cannabidiol on the acute memory and psychotomimetic effects of smoked cannabis: naturalistic study, The British Journal of Psychiatry, 201, 197:285-290), de Mello Schier et al., (Cannabidiol, a Cannabis sativa constituent, as an anxiolytic drug, Rev. Bras. Psiquiatr, 2012, 34(S1):5104-5117), and Zhornitsky et al. (Cannabidiol in Humans—the Quest for Therapeutic Targets, Pharmaceuticals, 2012, 5:529-552), each of which is herein incorporated by reference in its entirety for all purposes. Please see Table 1 for a non-limiting list of medical uses for cannabinoids.

TABLE 1
Non-limiting list of medical uses for cannabinoids.

| | MEDICAL | | |
|---|---|---|---|
| | USES | CANNABINOID | REFERENCES |
| 1 | Dystonia, | CBD | (a) Consroe, 1986, The |
| | Akathisia | | International journal of |
| | (Anti | | neuroscience 30 (4): 277-282 |
| | convulsant) | | (b) Snider et al., 1985, |
| | | | Neurology, (Suppl 1): 201. |
| 2 | Glaucoma | CBD | (a) Colasanti et al, Exp. Eye Res, |
| | | | 30:251-259, 1984 |
| | (lowers | CBG | (b) Gen. Pharmac. 15:479-484, |
| | | | 1984 |
| | intraocular | | (c) Craig et al. 1984, |
| | pressure) | | Experimental eye research 39 |
| | | | (3):251-259 |
| 3 | Ischemic | CBD | (a) U.S. Pat. No. 6,630,507 |
| | disease | | (b) Snider et al., 1985, |
| | (Alzheimer's, | | “Beneficial and Adverse Effects |
| | Parkinson's, | | of Cannabidiol in a Parkinson |
| | Down | | Patient with Sinemet-Induced |
| | Syndrome, | | Dystonic Dyskinesia”. |
| | HIV, | | Neurology, (Suppl 1): 201. |
| | Dementia) | | |
| 4 | Good for | CBD | (a) U.S. Pat. No. 6,630,507 |
| | patients | | |
| | treated with | | |
| | oxidant- | | |
| | inducing | | |
| | agents for | | |
| | chemotherapy, | | |
| | radiation. | | |
| 5 | Motion | CBD | U.S. Pat. No. 8,034,843 GW |
| | Sickness | | Pharma experiments on Shrews |
| | (Anti-emetic) | | (b) Mechoulam, et al., 2007, |
| | | | Chemistry & Biodiversity 4 (8): |
| | | | 1678-1692. |
| 6 | Pain-Brachial | THC | (a) US 20060135599 GW |
| | plexus | THC:CBD | Pharma |
| | avulsion | | |
| 7 | Pain and | CBD:THC | (a) US20080139667 |
| | inflammation- | | (b) Mechoulam, et al., 2007, |
| | Arthritis | | Chemistry & Biodiversity 4 (8): |
| | | | 1678-1692. |
| 8 | Anti Cancer- | CBD:THC | (a) US20080262099 |
| | cell movement | CBD | (b) Mechoulam, et al., 2007, |
| | | | Chemistry & Biodiversity 4 (8): |
| | | | 1678-1692. |
| | | | (c) McAllister et al., 2007, Mol. |
| | | | Cancer Ther. 6 (11): 2921-7. |
| 9 | Anti | CBDV | (a) US20120004251 |
| | Convulsant | CBD | (b) US20120165402 |
| | (against | | (c) Mechoulam, et al., 2007, |
| | seizures) | | Chemistry & Biodiversity 4 (8): |
| | | | 1678-1692. |
| | | | (d) Carlini et al., J. Clin. |
| | | | Pharmacol. 21:417S-427S, 1981 |
| | | | (e) Karler et al., J. Clin. |
| | | | Pharmacol. 21:437S-448S, 1981 |
| | | | (f) Consroe et al., J. Clin |
| | | | Pharmacol. 21:428S-436S, 1981 |
| 10 | Neurological | THC:CBD | (a) US20100035978 |
| | Pain (MS | | |
| | related) | | |
| 11 | Weight loss | THCV | (a) US20090306221 |
| | | | (b) US20080119544 |
| 12 | Anti- | CBG | (a) US20080031977 |
| | Depressant | | (b) US 60/813,814 |
| 13 | Irritable Bowel | THC:CBD | (a) EP 1361864 |
| | Syndrome | | (b) EP 1542657 |
| | (Crohn's) | | (c) US20100286098 |
| 14 | Type II | THCV:CBD | (a) US20110082195 |
| | diabetes | | |
| 15 | Anti-Psychotic | THCV:CBD | (a) US20110038958 |
| | | | (b) Zuardi et al., 2006, Braz. J. |
| | | | Med. Biol. Res. 39 (4): 421-429. |
| 16 | Cancer Pain | THC:CBD | (a) US20110230549 |
| 17 | Anxiety | CBD | (a) Mecnoulam, et al., 2007, |
| | Reduction | | Chemistry & Biodiversity 4 (8): |
| | | | 1678-1692. |
| | | | (b) Bergamaschi et al., 2003, |
| | | | Neuropsychopharmacology 36 |
| | | | (6): 1219-1226 |

### Cannabinoid Biosynthetic Pathways

The biosynthetic pathway of cannabinoids has been studied in great detail. See de Meijer et al. I, II, III, and IV (I: 2003, Genetics, 163:335-346; II: 2005, Euphytica, 145:189-198; III: 2009, Euphytica, 165:293-311; and IV: 2009, Euphytica, 168:95-112), each of which is herein incorporated by reference in its entirety for all purposes. According to the current model, phenolic precursors such as geranyl pyrophosphate (GPP) and polyketide, olivetolic acid (OA) are condensed by geranyl pyrophosphate olivetolate geranyl transferase (GOT) to form cannabigerolic acid (CBGA). Alternatively, GPP and divarinic acid can be condensed by GOT to form cannabigerovarinic acid (CBGVA). CBGA or CBGVA are considered to be the “primary cannabinoids” from which others can be produced.

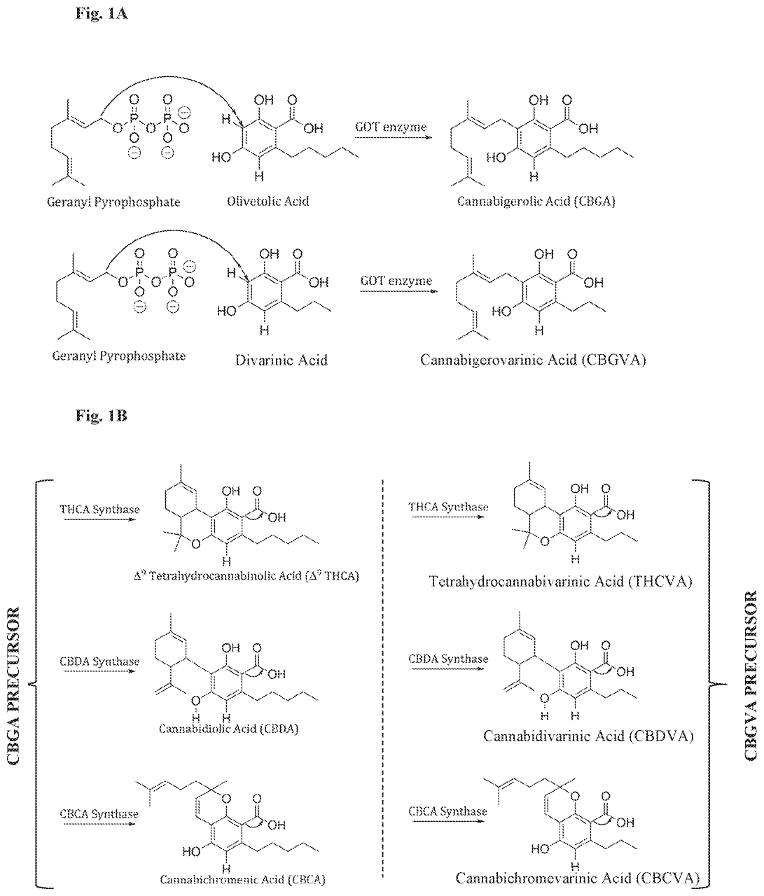

CBGA/CBGVA is quickly transformed in plants into, for example: (1) CBCA/CBCVA by CBCA synthase; (2) THCA/THCVA by THCA synthase; or (3) CBDA/CBDVA by CBDA synthase. See FIGS. 1A and B for a visual representation of the current model of cannabinoid biosynthesis. The genes coding for THCA synthase and CBDA synthase are found on the same B locus. Thus cannabis plants can be categorized into THC-CBD chemotypes based on the state of the B locus BT/BT (THC producing, chemotype I), BD % BD (CBD producing, chemotype III), and BT/BD (producing both THC and CBD, chemotype II). Additional information on the genetic regulation of cannabinoids can be found in de Meijer et al. I, II, III, and IV (1: 2003, Genetics, 163:335-346; II: 2005, Euphytica, 145:189-198; III: 2009, Euphytica, 165:293-311; and IV: 2009, Euphytica, 168:95-112). The BT and BD alleles are known, and can be easily detected using methods known to those skilled in the art, including Northerns, PCR, sequencing, or Westerns. A representative sequence of THCA synthase is available at GenBank ID AB057805.1. A representative sequence of the CBDA synthase is available at GenBank ID AB292682.1.

Cannabis Chemistry—Terpenes and Terpenoids, and other Volatiles

In some embodiments, the specialty plants and compositions of the present disclosure comprise novel Terpene Profiles. Terpenes are a large and diverse class of organic compounds, produced by a variety of plants. They are often strong smelling and thus may have had a protective function. Terpenes are derived biosynthetically from units of isoprene, which has the molecular formula C₅H₈. The basic molecular formulae of terpenes are multiples of (C₅H₈)ₙ where n is the number of linked isoprene units. The isoprene units may be linked together “head to tail” to form linear chains or they may be arranged to form rings. Non-limiting examples of terpenes include Hemiterpenes, Monoterpenes, Sesquiterpenes, Diterpenes, Sesterterpenes, Triterpenes, Sesquarterpenes, Tetraterpenes, Polyterpenes, and Norisoprenoids.

In addition to cannabinoids, cannabis also produces over 120 different terpenes (Russo 2011, Taming THC: potential cannabis synergy and phytocannabinoid-terpenoid entourage effects, British Journal of Pharmacology, 163:1344-1364). Within the context and verbiage of this document the terms ‘terpenoid’ and ‘terpene’ are used interchangeably.

Cannabinoids are odorless, so terpenoids are responsible for the unique odor of cannabis, and each variety has a slightly different profile that can potentially be used as a tool for identification of different varieties or geographical origins of samples (Hillig 2004. “A chemotaxonomic analysis of terpenoid variation in Cannabis” Biochem System and Ecology 875-891). Indeed, recent studies have concluded that terpene production in cannabis plants is strongly inherited, and is little influenced by environmental factors. (Casano et al 2011. “Variations in terpene profiles of different strains of Cannabis sativa” Acta Horticulturae 925:115-121). Accordingly, the development of novel terpene profiles requires the development of new genetics though traditional breeding or other techniques for genetic manipulation.

Terpenes also provide a unique and complex organoleptic profile for each variety that is appreciated by both novice users and connoisseurs. Critical differences between many popular commercial cannabis strains can be largely attributed to differences in Terpene Profiles, which provide each line with their distinctive aroma and pharmacological effects. The popular “cookies” strain of cannabis, is noted by its myrcene and limonene. In addition to many circulatory and muscular effects, some terpenes interact with neurological receptors. A few terpenes produced by cannabis plants also bind weakly to cannabinoid receptors. Some terpenes can alter the permeability of cell membranes and allow in either more or less THC, while other terpenes can affect serotonin and dopamine chemistry as neurotransmitters. Terpenoids are lipophilic, and can interact with lipid membranes, ion channels, a variety of different receptors (including both G-protein coupled odorant and neurotransmitter receptors), and enzymes. Some are capable of absorption through human skin and passing the blood brain barrier.

Generally speaking, terpenes are considered to be pharmacologically relevant when present in concentrations of at least 0.05% in plant material (Hazekamp and Fischedick 2010. “Metabolic fingerprinting of Cannabis sativa L., cannabinoids and terpenoids for chemotaxonomic and drug standardization purposes” Phytochemistry 2058-73; Russo 2011, Taming THC: potential cannabis synergy and phytocannabinoid-terpenoid entourage effects, British Journal of Pharmacology, 163:1344-1364). Thus, although there are an estimated 120 different terpenes, only a few are produced at high enough levels to be detectable, and fewer still which are able to reach organoleptic or pharmacologically relevant levels.

Terpenoids can be extracted from the plant material by steam distillation (giving you essential oil) or vaporization, however the yield varies greatly by plant tissue, type of extraction, age of material, and other variables (McPartland and Russo 2001 “Cannabis and Cannabis Extracts: Greater Than the Sum of Their Parts?” Hayworth Press). In some embodiments, the present disclosure teaches methods for extracting cannabinoids and terpenes. Other methods for producing reproducible and quantifiable cannabinoid and terpene measurements are known to persons having skill in the art. Typically, the yield of terpenoids in cannabis inflorescences is less than 2% by weight on analysis; however, it is thought that they may comprise up to 10% of the trichome content. A few of the most recognized terpenes and non-terpene volatiles in cannabis are discussed below.

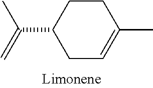

D-Limonene, also known as limonene, is a monoterpenoid that is widely distributed in nature and often associated with citrus. It has strong anxiolytic properties in both mice and humans, apparently increasing serotonin and dopamine in mouse brain. D-limonene has potent antidepressant activity when inhaled. It is also under investigation for a variety of different cancer treatments, with some focus on its hepatic metabolite, perillic acid. There is evidence for activity in the treatment of dermatophytes and gastro-oesophageal reflux, as well as having general radical scavenging properties (Russo 2011, Taming THC: potential cannabis synergy and hvtocannabinoid-terpenoid entourage effects British Journal of Pharmacology, 163:1344-1364).

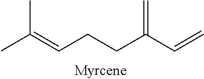

β-Myrcene, also known as myrcene, is a monoterpenoid also found in cannabis, and has a variety of pharmacological effects. It is often associated with a sweet fruit like taste. It reduces inflammation, aids sleep, and blocks hepatic carcinogenesis, as well as acting as an analgesic and muscle relaxant in mice. When f-myrcene is combined with Δ9-THC it could intensify the sedative effects of Δ9-THC, causing the well-known “couch-lock” effect that some cannabis users experience (Russo 2011, Taming THC: potential cannabis synergy and phytocannabinoid-terpenoid entourage effects, British Journal of Pharmacology, 163:1344-1364).

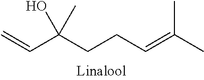

D-Linalool, also known as linalool, is a monoterpenoid with very well-known anxiolytic effects. It is often associated with lavender, and frequented used in aromatherapy for its sedative impact. It acts as a local anesthetic and helps to prevent scarring from burns, is anti-nociceptive in mice, and shows anti-glutamatergic and anticonvulsant activity. Its effects on glutamate and GABA neurotransmitter systems are credited with giving it its sedative, anxiolytic, and anticonvulsant activities (Russo 2011, Taming THC: potential cannabis synergy and phytocannabinoid-terpenoid entourage effects, British Journal of Pharmacology, 163:1344-1364).

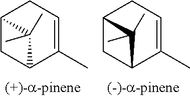

α-Pinene is a monoterpene common in nature, also with a plethora of effects on mammals and humans. It acts as an acetylcholinesterase inhibitor, which aids memory and counteracts the short-term memory loss associated with Δ₉-THC intoxication, is an effective antibiotic agent, and shows some activity against MRSA. In addition, a-pinene is a bronchodilator in humans and has anti-inflammatory properties via the prostaglandin E-1 pathway (Russo 2011, Taming THC: potential cannabis synergy and phytocannabinoid-terpenoid entourage effects, British Journal of Pharmacology, 163:1344-1364).

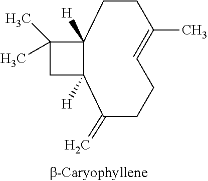

β-Caryophyllene is often the most predominant sesquiterpenoid in cannabis. It is less volatile than the monoterpenoids, thus it is found in higher concentrations in material that has been processed by heat to aid in decarboxylation. It is very interesting in that it is a selective full agonist at the CB₂ receptor, which makes it the only phytocannabinoid found outside the cannabis genus. In addition, it has anti-inflammatory and gastric cytoprotective properties, and may even have anti-malarial activity.

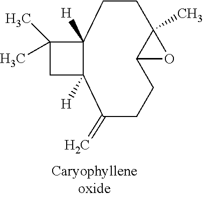

Caryophyllene oxide is another sesquiterpenoid found in cannabis, which has antifungal and anti-platelet aggregation properties. As an aside, it is also the molecule that drug-sniffing dogs are trained to find (Russo 2011, Taming THC: potential cannabis synergy and phytocannabinoid-terpenoid entourage effects, British Journal of Pharmacology, 163:1344-1364).

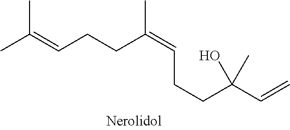 [SMILES: C=CC(C)(O)CC/C=C(/C)CCC=C(C)C]

Nerolidol is a sesquiterpene that is often found in citrus peels that exhibits a range of interesting properties. It acts as a sedative, inhibits fungal growth, and has potent anti-malarial and antileishmanial activity. It also alleviated colon adenomas in rats (Russo 2011, Taming THC: potential cannabis synergy and phytocannabinoid-terpenoid entourage effects, British Journal of Pharmacology, 163:1344-1364). Phytol is a diterpene often found in cannabis extracts. It is a degradation product of chlorophyll and tocopherol. It increases GABA expression and therefore could be responsible the relaxing effects of green tea and wild lettuce. It also prevents vitamin-A induced teratogenesis by blocking the conversion of retinol to its dangerous metabolite, all-trans-retinoic acid (Russo 2011, Taming THC: potential cannabis synergy and phytocannabinoid-terpenoid entourage effects, British Journal of Pharmacology, 163:1344-1364).

Some of the most commonly found terpenoids in cannabis are summarized in Table 2, with their individual organoleptic properties as well as their basic pharmacology.

TABLE 2
A non-limiting list of the medical effects of some of the most common
terpenes found in cannabis

| | Odor | | Suggested |
|---|---|---|---|
| Terpenoid | Description | Flavor Description | Pharmacology |
| a-pinene | Herbal, | Woody, piney, | Anti-inflammatory, |
| | piney | camphoraceous | bronchodilator, |
| | | | stimulant |
| camphene | Woody, | Camphoraceous, | Reduces plasma |
| | piney | cooling, minty | cholesterol and |
| | | | triglycerides, |
| | | | Antioxidant and free |
| | | | radical scavenger |
| b-pinene | Herbal, | Fresh, piney, | Strong antimicrobial |
| | cooling, | woody | |
| | piney | | |
| myrcene | Spicy, | Woody, | Anti-inflammatory, |
| | herbaceous | vegetative, citrus | sedative, antibiotic, |
| | | | analgesic |
| a- | Terpenic, | Terpenic, citrus, | Antinociceptive |
| phellandrene | citrus | lime | |
| carene | Citrus, | None given | CNS depressant, |
| | sweet | | anti---inflammatory |
| a-terpinene | Woody, | Terpenic, woody, | Antioxidant |
| | citrus, | piney | |
| | medicinal | | |
| limonene | Citrus, | Sweet, orange, | Anxiolytic, |
| | fresh | citrus | antidepressant, |
| | | | immunostimulant |
| b-ocimene | Floral, | Green, tropical, | Possible anti---bacterial |
| | green | woody | |
| g-terpinene | Terpenic, | Terpenic, citrus, | Antioxidant |
| | woody | lime-like | |
| terpinolene | Herbal, | Sweet, fresh, | Comforting, calming, |
| | woody | piney, citrus | anti-oxidant, antifungal |
| linalool | Floral, | Citrus, orange, | Sedative, anxiolytic, |
| | citrus | lemon, floral | immunostimulant |
| fenehol | Camphor, | Fresh, piney | Possible stimulant |
| | piney | | |
| a-terpineol | Floral, | None given | Sedative, AChE |
| | piney | | inhibitor, antioxidant |
| b- | Spicy, | Spicy, clove, | Selective agonist of |
| caryophyllene | woody | rosemary | CB2 receptor, anti- |
| | | | inflammatory, |
| | | | antimalarial |
| a-humulene | Woody | None given | Anti-inflammatory |
| caryophyllene | Woody, | None given | Antifungal, stimulant |
| oxide | sweet | | |

### Analysis of Cannabinoids and Terpenes

As reported herein, the absolute cannabinoid and terpene contents of a plant are calculated based on weight of cannabinoid or terpene present in a sample divided by the dried weight of the dried trimmed inflorescence. Dried inflorescences refer to harvested inflorescence tissue dried to ˜10% moisture level. Where specifically indicated, terpene and cannabinoid contents are further adjusted to account for any remaining moisture content, by removing the weight of any remaining moisture from the measured weight of the inflorescence. Moisture content of a flower can be determined by a variety of analytical methods. Persons having skill in the art will be familiar with methods for measuring moisture content. In some embodiments, the present disclosure teaches the use of FTIR analysis for calculating moisture content of inflorescences. In other embodiments, the present disclosure teaches the use of additional drying steps in desiccant chambers to calculate remaining moisture contents.

The term trimmed inflorescence as used herein refers to inflorescences with sun (sugar) leaves cut off such that only the calyx and reproductive buds remain. Trimming can be performed manually, through careful manicuring of harvested tissue, or via automated mechanical methods.

In some embodiments, the present disclosure also teaches methods of pre-screening grown seeds for specific cannabinoid contents. For example, the types of cannabinoids produced by a cannabis inflorescence can also be determined in the field via thin layer chromatography (TLC) analysis (see “Cannabis Inflorescence & Leaf QC” from The American Herbal Pharmacopeia 2013).

The present disclosure will often refer to Specialty Cannabis comprising a selected cannabinoid or terpene content. In some instances, the present disclosure will refer to Specialty Cannabis that produces inflorescences comprising a selected cannabinoid or terpene content. It will be understood that both of these statements are interchangeable, and that references to the cannabinoid or terpene contents of a Specialty Cannabis refer to the contents of the inflorescences those plants produce.

### Specialty Cannabis

In some embodiments, the present disclosure provides Specialty Cannabis with novel cannabinoid profiles. The presently disclosed inventions are based in part on the instant inventors' discovery that cannabis plants could be bred to produce high levels of secondary cannabinoids, while also accumulating primary cannabinoid compounds. The presently disclosed cannabinoid profiles were highly unexpected, and run contrary to previously accepted limitations of cannabinoid biosynthetic models. The novelty of the presently disclosed invention is discussed in more detail below.

For the past several hundred years, cannabis cultivation and breeding dogmas have focused on the development of high THC-producing plants, often at the expense of most other cannabinoids. Only recently, has this single-minded approach been challenged, as emerging medical research has started to uncover the medicinal benefits of other non-THC cannabinoids. Seeking to capitalize on these results, breeders have started to develop plants with selected cannabinoids, such as CBD, THCV, and CBC. (See e.g. U.S. Pat. No. 9,095,554; US. Patent Publication No. US 2016/0360721, both of which are hereby incorporated by reference). Despite some early success, several cannabinoid combinations have proved elusive to breeders.

One of the most challenging cannabinoids to produce in cannabis plants is CBG. Because of its location in the cannabinoid biosynthetic pathway, CBG is typically only found in very low trace quantities, the vast majority of the compound having been converted to other cannabinoids by downstream enzymes in the plant. The only cannabis plants to have accumulated any appreciable CBG content were reported by De Meijer in 2005 (De Meijer and Hammond 2005, “The Inheritance of chemical phenotype in Cannabis sativa (II): Cannabigerol predominant plants” Euphytica 145:189-198). These plants were reported as having a B0/BO genotype, in which both the THCA and CBDA synthase enzymes had been rendered non-functional. De Meijer's plants accumulated as high as 6% CBGA (not max) quantities, but did so at the expense of all other cannabinoids, save for small 1-2% CBDA (not max) accumulation attributed to residual activity in the B0 allele.

De Meijer reported however, that the presence of any functional THCA synthase or CBDA synthase allele in F2 and F3 plants completely obliterated the CBG phenotype, resulting in the accumulation of only THCA or CBDA, respectively. These shortcomings in existing cannabis varieties posed serious hurdles to recreational consumers and medical patients that sought to the benefits of administering CBG together with one or more other secondary cannabinoids (e.g., CBG-THC, CBG-CBD, CBG-THCV, CBG-CBDV combinations).

In some embodiments, the Specialty Cannabis plants of the present disclosure represent a new category of cannabis plants in which high levels of primary cannabinoids CBG or CBGV accumulate while in the presence of one or more functional THCA synthase or CBDA synthase alleles. Thus, in some embodiments, the Specialty Cannabis plants of the present disclosure solve the problems of previously existing cannabis lines, by exhibiting cannabinoid profiles comprising high (non-trace) levels of primary cannabinoids CBG/CBGV together with high (non-trace) levels of one or more non-CBG cannabinoid(s)(“NGCs”). In some embodiments, the Specialty Cannabis of the present disclosure accumulate high (>2%, >3%, >4%, >5%) CBG and or CBGV, and high (>3%, >4%, >5%) total NGCs.

In some embodiments, the Specialty Cannabis of the present disclosure produce inflorescences comprising about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% total cannabinoids by weight of the dried inflorescence, and all ranges therebetween. Thus, in some embodiments, the Specialty Cannabis of the present disclosure comprise 1-40%, 1-30%, or 1-25% cannabinoid content by weight of the dried inflorescence.

In some embodiments, the Specialty Cannabis of the present disclosure produce inflorescences comprising more than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% total cannabinoids by weight of the dried inflorescence.

In some embodiments, the Specialty Cannabis of the present disclosure produce inflorescences comprising about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% CBG and/or CBGV by weight of the dried inflorescence, and all ranges therebetween. Thus, in some embodiments, the Specialty Cannabis of the present disclosure comprise 2%-10%, 3%-30%, or 3%-25% CBG and/or CBGV content by weight of the dried inflorescence.

In some embodiments, the Specialty Cannabis of the present disclosure produce inflorescences comprising more than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% CBG and/or CBGV by weight of the dried inflorescence.

In some embodiments, the Specialty Cannabis of the present disclosure produce inflorescences comprising about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% THC by weight of the dried inflorescence, and all ranges therebetween. Thus, in some embodiments, the Specialty Cannabis of the present disclosure comprise 3%-40%, 3-30%, or 3%-25% THC content by weight of the dried inflorescence.

In some embodiments, the Specialty Cannabis of the present disclosure produce inflorescences comprising more than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% THC by weight of the dried inflorescence.

In some embodiments, the Specialty Cannabis of the present disclosure produce inflorescences comprising CBG/CBGV together with non-trace quantities of THC. Thus, in some embodiments, the Specialty Cannabis of the present disclosure produce inflorescences comprising about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% THC by weight of the dried inflorescence, and all ranges therebetween, with non-trace quantities of CBG/CBGV.

Thus, in some embodiments, the Specialty Cannabis of the present disclosure produce inflorescences comprising more than about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% THC by weight of the dried inflorescence, with non-trace quantities of CBG/CBGV.

In some embodiments, the Specialty Cannabis of the present disclosure produce inflorescences comprising about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% CBD by weight of the dried inflorescence, and all ranges therebetween. Thus, in some embodiments, the Specialty Cannabis of the present disclosure comprise 3%-40%, 3%-30%, or 3%-25% CBD content by weight of the dried inflorescence.

In some embodiments, the Specialty Cannabis of the present disclosure produce inflorescences comprising more than about 1%, 2%, 3%, 4%, 5%, 6%, 7%, 8%, 90%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% CBD by weight of the dried inflorescence.

In some embodiments, the Specialty Cannabis of the present disclosure produce inflorescences comprising CBG/CBGV together with non-trace quantities of CBD. Thus, in some embodiments, the Specialty Cannabis of the present disclosure produce inflorescences comprising greater than about 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% CBD by weight of the dried inflorescence with non-trace quantities of CBG/CBGV, and all ranges therebetween.

In some embodiments, the Specialty Cannabis of the present disclosure produce inflorescences comprising greater than about 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% CBD by weight of the dried inflorescence with non-trace quantities of CBG/CBGV.

In some embodiments, the Specialty Cannabis of the present disclosure accumulates propyl cannabinoids (e.g., THCV and CBDV). Thus, in some embodiments, the Specialty Cannabis of the present disclosure produce inflorescences comprising about 1%, 2%, 3%, 4%, 5%, 6%, 7%, 8%, 90%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% total propyl cannabinoids by weight of the dried inflorescence, and all ranges therebetween. Thus, in some embodiments, the Specialty Cannabis of the present disclosure comprise 3%-40%, 3%-30%, or 3%-25% total propyl cannabinoid content by weight of the dried inflorescence.

In some embodiments, the Specialty Cannabis of the present disclosure produce inflorescences comprising more than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% total propyl cannabinoids by weight of the dried inflorescence.

In some embodiments, the Specialty Cannabis of the present disclosure produce inflorescences comprising CBG/CBGV together with non-trace quantities of propyl cannabinoids.

Thus, in some embodiments, the Specialty Cannabis of the present disclosure produce inflorescences comprising about 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% total propyl cannabinoid by weight of the dried inflorescence, and all ranges therebetween, with non-trace quantities of CBG/CBGV.

In some embodiments, the Specialty Cannabis of the present disclosure produce inflorescences comprising more than about 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% total propyl cannabinoid by weight of the dried inflorescence, with non-trace quantities of CBG/CBGV.

In some embodiments, the Specialty Cannabis of the present disclosure accumulates CBC.

Thus, in some embodiments, the Specialty Cannabis of the present disclosure produce inflorescences comprising about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% CBC by weight of the dried inflorescence, and all ranges therebetween. Thus, in some embodiments, the Specialty Cannabis of the present disclosure comprise 3%-40%, 3%-30%, or 3%-25% CBC content by weight of the dried inflorescence.

In some embodiments, the Specialty Cannabis of the present disclosure produce inflorescences comprising more than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% CBC by weight of the dried inflorescence.

In some embodiments, the Specialty Cannabis of the present disclosure produce inflorescences comprising CBG/CBGV together with non-trace quantities of CBC. Thus, in some embodiments, the Specialty Cannabis of the present disclosure produce inflorescences comprising about 3%, 4%, 5%, 6%, 7%, 8%, 9%, 10%, 11%, 12%, 13%, 14%, 15%, 16%, 17%, 18%, 19%, or 20% CBC by weight of the dried inflorescence, and all ranges therebetween, with non-trace quantities of CBG/CBGV.

In some embodiments, the Specialty Cannabis of the present disclosure produce inflorescences comprising more than about 3%, 4%, 5%, 6%, 7%, 8%, 9%, 10%, 11%, 12%, 13%, 14%, 15%, 16%, 17%, 18%, 19%, or 20% CBC by weight of the dried inflorescence with non-trace quantities of CBG/CBGV.

In some embodiments, the Specialty Cannabis of the present disclosure produce inflorescences comprising about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% THCV by weight of the dried inflorescence, and all ranges therebetween. Thus, in some embodiments, the Specialty Cannabis of the present disclosure comprise 3%-40%, 3%-30%, or 3%-25% THCV content by weight of the dried inflorescence.

In some embodiments, the Specialty Cannabis of the present disclosure produce inflorescences comprising more than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% THCV by weight of the dried inflorescence.

In some embodiments, the Specialty Cannabis of the present disclosure produce inflorescences comprising CBG/CBGV together with non-trace quantities of THCV. Thus, in some embodiments, the Specialty Cannabis of the present disclosure produce inflorescences comprising about 2%, 3%, 4%, 5%, 6%, 7%, 8%, 9%, 10%, 11%, 12%, 13%, 14%, 15%, 16%., 17%, 18%, 19%, or 20% THCV by weight of the dried inflorescence, and all ranges therebetween, with non-trace quantities of CBG/CBGV.

In some embodiments, the Specialty Cannabis of the present disclosure produce inflorescences comprising more than about 2%, 3%, 4%, 5%, 6%, 7%, 8%, 9%, 10%, 11%, 12%, 13%, 14%, 15%, 16%, 17%, 18%, 19%, or 20% THCV by weight of the dried inflorescence with non-trace quantities of CBG/CBGV.

In some embodiments, the Specialty Cannabis of the present disclosure produce inflorescences comprising about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% CBDV by weight of the dried inflorescence, and all ranges therebetween. Thus, in some embodiments, the Specialty Cannabis of the present disclosure comprise 3%-40%, 3%-30%, or 3%-25% CBDV content by weight of the dried inflorescence.

In some embodiments, the Specialty Cannabis of the present disclosure produce inflorescences comprising more than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% CBDV by weight of the dried inflorescence.

In some embodiments, the Specialty Cannabis of the present disclosure produce inflorescences comprising CBG/CBGV together with non-trace quantities of CBDV. Thus, in some embodiments, the Specialty Cannabis of the present disclosure produce inflorescences comprising about 2%, 3%, 4%, 5%, 6%, 7%, 8%, 9%, 10%, 11%, 12%, 13%, 14%, 15%, 16%, 17%, 18%, 19%, or 20% CBDV by weight of the dried inflorescence, and all ranges therebetween, with non-trace quantities of CBG/CBGV.

In some embodiments, the Specialty Cannabis of the present disclosure produce inflorescences comprising more than about 2%, 3%, 4%, 5%, 6%, 7%, 8%, 9%, 10%, 11%, 12%, 13%, 14%, 15%, 16%, 17%, 18%, 19%, or 20% CBDV by weight of the dried inflorescence with non-trace quantities of CBG/CBGV.

In some embodiments, the Specialty Cannabis of the present disclosure produce inflorescences comprising about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% CBCV by weight of the dried inflorescence, and all ranges therebetween. Thus, in some embodiments, the Specialty Cannabis of the present disclosure comprise 3%-40%, 3%-30%, or 3%-25% CBCV content by weight of the dried inflorescence.

In some embodiments, the Specialty Cannabis of the present disclosure produce inflorescences comprising more than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% CBCV by weight of the dried inflorescence.

In some embodiments, the Specialty Cannabis of the present disclosure produce inflorescences comprising CBG/CBGV together with non-trace quantities of CBCV. Thus, in some embodiments, the Specialty Cannabis of the present disclosure produce inflorescences comprising about 2%, 3%, 4%, 5%, 6%, 7%, 8%, 9%, 10%, 11%, 12%, 13%, 14%, 15%, 16%, 17%, 18%, 19%, or 20% CBCV by weight of the dried inflorescence, and all ranges therebetween, with non-trace quantities of CBG/CBGV.

In some embodiments, the Specialty Cannabis of the present disclosure produce inflorescences comprising more than about 2%, 3%, 4%, 5%, 6%, 7%, 8%, 9%, 10%, 11%, 12%, 13%, 14%, 15%, 16%, 17%, 18%, 19%, or 20% CBCV by weight of the dried inflorescence with non-trace quantities of CBG/CBGV.

Indeed, in some embodiments, the Specialty Cannabis of the present disclosure produce inflorescences comprising about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% of total NGCs by weight of the dried inflorescence, and all ranges therebetween.

Another important aspect of cannabis breeding is the Terpene Profile of a plant. In some embodiments, the present invention teaches the preference for cannabis plant material with novel Terpene Profiles. In some embodiments, the Specialty Cannabis of the present disclosure produce inflorescences comprising organoleptically pleasing Terpene Profiles.

In some embodiments, the Specialty Cannabis of the present invention has an absolute content of any one of the 17 terpenes in the Terpene Profile that is 0%, 0.01%, 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99%, 1%, 1.01%, 1.02%, 1.03%, 1.04%, 1.05%, 1.06%, 1.07%, 1.08%, 1.09%, 1.1%, 1.11%, 1.12%, 1.13%, 1.14%, 1.15%, 1.16%, 1.17%, 1.18%, 1.19%, 1.2%, 1.21%, 1.22%, 1.23%, 1.24%, 1.25%, 1.26%, 1.27%, 1.28%, 1.29%, 1.3%, 1.31%, 1.32%, 1.33%, 1.34%, 1.35%, 1.36%, 1.37%, 1.38%, 1.39%, 1.4%, 1.41%, 1.42%, 1.43%, 1.44%, 1.45%, 1.46%, 1.47%, 1.48%, 1.49%, 1.5%, 1.51%, 1.52%, 1.53%, 1.54%, 1.55%, 1.56%, 1.57%, 1.58%, 1.59%, 1.6%, 1.61%, 1.62%, 1.63%, 1.64%, 1.65%, 1.66%, 1.67%, 1.68%, 1.69%, 1.7%, 1.71%, 1.72%, 1.73%, 1.74%, 1.75%, 1.76%, 1.77%, 1.78%, 1.79%, 1.8%, 1.81%, 1.82%, 1.83%, 1.84%, 1.85%, 1.86%, 1.87%, 1.88%, 1.89%, 1.9%, 1.91%, 1.92%, 1.93%, 1.94%, 1.95%, 1.96%, 1.97%, 1.98%, 1.99%, 2%, 2.01%, 2.02%, 2.03%, 2.04%, 2.05%, 2.06%, 2.07%, 2.08%, 2.09%, 2.1%, 2.11%, 2.12%, 2.13%, 2.14%, 2.15%, 2.16%, 2.17%, 2.18%, 2.19%, 2.2%, 2.21%, 2.22%, 2.23%, 2.24%, 2.25%, 2.26%, 2.27%, 2.28%, 2.29%, 2.3%, 2.31%, 2.32%, 2.33%, 2.34%, 2.35%, 2.36%, 2.37%, 2.38%, 2.39%, 2.4%, 2.41%, 2.42%, 2.43%, 2.44%, 2.45%, 2.46%, 2.47%, 2.48%, 2.49%, 2.5%, 2.51%, 2.52%, 2.53%, 2.54%, 2.55%, 2.56%, 2.57%, 2.58%, 2.59%, 2.6%, 2.61%, 2.62%, 2.63%, 2.64%, 2.65%, 2.66%, 2.67%, 2.68%, 2.69%, 2.7%, 2.71%, 2.72%, 2.73%, 2.74%, 2.75%, 2.76%, 2.77%, 2.78%, 2.79%, 2.8%, 2.81%, 2.82%, 2.83%, 2.84%, 2.85%, 2.86%, 2.87%, 2.88%, 2.89%, 2.9%, 2.91%, 2.92%, 2.93%, 2.94%, 2.95%, 2.96%, 2.97%, 2.98%, 2.99%, 3%, 3.2%, 3.4%, 3.6%, 3.8%, 4%, 4.2%, 4.3%, 4.4%, 4.6%, 4.8%, 5%, 5.2%, 5.4%, 5.6%, 5.8%, 6%, 6.2%, 6.4%, 6.6%, 6.8%, 7%, 7.2%, 7.4%, 7.6%, 7.8%, 8%, or greater based on dry weight of inflorescence, including all ranges therebetween. Thus in some embodiments the absolute content of any one of the terpenes is between about 0.05% and about 0.85%. This paragraph is intended to be read as applying to any specific terpene(s) in a Terpene Profile, such that the name of any one or two or more of these terpenes as specifically referred to elsewhere herein (e.g., linalool) can replace the phrase “any one of the 17 terpenes in the Terpene Profile.”

In some embodiments, the Specialty Cannabis of the present invention has an absolute content of any one of the 17 terpenes in the Terpene Profile that is greater than 0%, 0.01%, 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%0, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99%, 1%, 1.01%, 1.02%, 1.03%, 1.04%, 1.05%, 1.06%, 1.07%, 1.08%, 1.09%, 1.1%, 1.11%, 1.12%, 1.13%, 1.14%, 1.15%, 1.16%, 1.17%, 1.18%, 1.19%, 1.2%, 1.21%, 1.22%, 1.23%, 1.24%, 1.25%, 1.26%, 1.27%, 1.28%, 1.29%, 1.3%, 1.31%, 1.32%, 1.33%, 1.34%, 1.35%, 1.36%, 1.37%, 1.38%, 1.39%, 1.4%, 1.41%, 1.42%, 1.43%, 1.44%, 1.45%, 1.46%, 1.47%, 1.48%, 1.49%, 1.5%, 1.51%, 1.52%, 1.53%, 1.54%, 1.55%, 1.56%, 1.57%, 1.58%, 1.59%, 1.6%, 1.61%, 1.62%, 1.63%, 1.64%, 1.65%, 1.66%, 1.67%, 1.68%, 1.69%, 1.7%, 1.71%, 1.72%, 1.73%, 1.74%, 1.75%, 1.76%, 1.77%, 1.78%, 1.79%, 1.8%, 1.81%, 1.82%, 1.83%, 1.84%, 1.85%, 1.86%, 1.87%, 1.88%, 1.89%, 1.9%, 1.91%, 1.92%, 1.93%, 1.94%, 1.95%, 1.96%, 1.97%, 1.98%, 1.99%, 2%, 2.01%, 2.02%, 2.03%, 2.04%, 2.05%, 2.06%, 2.07%, 2.08%, 2.09%, 2.1%, 2.11%, 2.12%, 2.13%, 2.14%, 2.15%, 2.16%, 2.17%, 2.18%, 2.19%, 2.2%, 2.21%, 2.22%, 2.23%, 2.24%, 2.25%, 2.26%, 2.27%, 2.28%, 2.29%, 2.3%, 2.31%, 2.32%, 2.33%, 2.34%, 2.35%, 2.36%, 2.37%, 2.38%, 2.39%, 2.4%, 2.41%, 2.42%, 2.43%, 2.44%, 2.45%, 2.46%, 2.47%, 2.48%, 2.49%, 2.5%, 2.51%, 2.52%, 2.53%, 2.54%, 2.55%, 2.56%, 2.57%, 2.58%, 2.59%, 2.6%, 2.61%, 2.62%, 2.63%, 2.64%, 2.65%, 2.66%, 2.67%, 2.68%, 2.69%, 2.7%, 2.71%, 2.72%, 2.73%, 2.74%, 2.75%, 2.76%, 2.77%, 2.78%, 2.79%, 2.8%, 2.81%, 2.82%, 2.83%, 2.84%, 2.85%, 2.86%, 2.87%, 2.88%, 2.89%, 2.9%, 2.91%, 2.92%, 2.93%, 2.94%, 2.95%, 2.96%, 2.97%, 2.98%, 2.99/c, 3%, 3.2%, 3.4%, 3.6%, 3.8%, 4%, 4.2%, 4.3%, 4.4%, 4.6%, 4.8%, 5%, 5.2%, 5.4%, 5.6%, 5.8%, 6%, 6.2%, 6.4%, 6.6%, 6.8%, 7%, 7.2%, 7.4%, 7.6%, 7.8%, or 8% based on dry weight of inflorescence.

This paragraph is intended to be read as applying to any specific terpene(s) in a Terpene Profile, such that the name of any one or two or more of these terpenes as specifically referred to elsewhere herein (e.g., linalool) can replace the phrase “any one of the 17 terpenes in the Terpene Profile.”

A limonene dominant terpene is used to refer to Terpene Profiles in which limonene is the most abundant terpene in the Terpene Profile (i.e., limonene relative or absolute content is >content of any single one of the 16 other terpenes in the Terpene Profile). Reference to other dominant terpenes is similarly based on said terpene being the most abundant within the Terpene Profile.

In some embodiments, the Specialty Cannabis of the present invention comprises 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8% terpene essential oil content by dry weight, including all ranges therebetween. Thus in some embodiments the essential oil content of the Specialty Cannabis varieties of the present invention is between about 0.5% and about 8% by dry weight. In other embodiments the essential oil contents of the Specialty Cannabis varieties of the present invention is between about 1.0% and about 5% by dry weight.

In some embodiments, the Specialty Cannabis of the present invention has greater than 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, or 8% terpene essential oil content by weight of the dried inflorescence.

In some embodiments, the terpene content of the Specialty Cannabis of the present disclosure is described in relative terms as a percentage composition of the total Terpene Profile. Thus for example a Specialty Cannabis with 1.2% absolute terpinolene content and 1.2% limonene content and no other terpenes in the Terpene Profile would said to have 50% terpinolene and 50% limonene relative content.

In some embodiments, the Specialty Cannabis of the present invention has a relative content of any one of the 17 terpenes in the Terpene Profile that is greater than or less than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 79%, 79%, 80%, 81%, 82%, 83%, 84%, 85%, 86%, 87%, 88%, 89%, or 90%, 91%, 92%, 93%, 94%, 95%, 96%, 97%, 98%, 99%, or 100%, including any ranges therebetween. Thus in some embodiments the relative content of any one of the terpenes is between 0% and 100%. This paragraph is intended to be read as applying to any specific terpene(s) in a Terpene Profile, such that the name of any of one or two or more these terpenes as specifically referred to elsewhere herein (e.g., linalool) can replace the phrase “any one of the 17 terpenes in the Terpene Profile.”

In some embodiments, the Specialty Cannabis of the present disclosure produce female inflorescences. In some embodiments, the Specialty Cannabis of the present disclosure have been feminized to produce female seed. In some embodiments, the supporting seed deposits referenced in the present disclosure are feminized. Persons having skill in the art will be familiar with techniques to feminize cannabis seeds, including breeding through treatment with silver thiosulfate, colloidal silver, hormones, and rodelization method.

Another important breeding phenotype is flower color. The accumulation of anthocyanins, carotenoids, or other color-producing compounds in leaves and flowers of cannabis can have an effect on consumer visual appeal and flavor. Iconic examples of the appeal of color are the popular “Purple Kush”, “Purple Haze”, and “Purple Trainwreck” varieties that express anthocyanins in their late maturation stages to produce dark purple leaves. Color selections can also be based on (but not limited to) unique coloration of stem, leaf, inflorescence, calyx, stamen, trichome bodies and finished products including extracts and hash.

Yield is another important factor in breeding. Cannabis yield is measured by pounds (lbs), grams (g) or kilograms (Kg) of dried (e.g., ˜10% moisture) trimmed flowers. Yield can be expressed in terms of yield per plant, yield per watt of light, and yield per square meter of growing area among others. Cannabis yield is also dependent on the growing environment. For example, yields for a particular cannabis strain will vary between outdoor growth long season, outdoor growth short season, or indoor growth. Yield may also be affected by growing conditions such as type of lighting, soil, fertilizer use, size of growing pot, etc.

In some embodiments, the Specialty Cannabis of the present disclosure produce 0.1 g, 0.2 g, 0.3 g, 0.4 g, 0.5 g, 0.6 g, 0.7 g, 0.8 g, 0.9 g, 1.0 g, 1.1 g, 1.2 g, 1.3 g, 1.4 g, 1.5 g, 1.6 g, 1.7 g, 1.8 g, 1.9 g, 2.0 g, 2.1 g, 2.2 g, 2.3 g, 2.4 g, 2.5 g, 2.6 g, 2.7 g, 2.8 g, 2.9 g, 3.0 g, 3.1 g, 3.2 g, 3.3 g, 3.4 g, 3.5 g, 3.6 g, 3.7 g, 3.8 g, 3.9 g, 4.0 g, 4.1 g, 4.2 g, 4.3 g, 4.4 g, 4.5 g, 4.6 g, 4.7 g, 4.8 g, 4.9 g, or 5.0 g of dried flowers per watt of light, including all ranges therebetween. In some embodiments, the Specialty Cannabis of the present invention produces 10 g, 15 g, 20 g, 25 g, 30 g, 35 g, 40 g, 45 g, 50 g, 55 g, 60 g, 65 g, 70 g, 75 g, 80 g, 85 g, 90 g, 95 g, 100 g, 105 g, 110 g, 115 g, 120 g, 125 g, 130 g, 135 g, 140 g, 145 g, 150 g, 155 g, 160 g, 165 g, 170 g, 175 g, 180 g, 185 g, 190 g, 195 g, 200 g, 210 g, 220 g, 230 g, 240 g, 250 g, 260 g, 270 g, 280 g, 290 g, 300 g, 310 g, 320 g, 330 g, 340 g, 350 g, 360 g, 370 g, 380 g, 390 g, 400 g, 410 g, 420 g, 430 g, 440 g, 450 g, 460 g, 470 g, 480 g, 490 g, 500 g, 550 g, 600 g, 650 g, 700 g, 750 g, 800 g, 850 g, 900 g, 950 g, 1000 g, 2000 g, 3000 g, or 5000 g of dried flowers per plant, including all ranges therebetween.

Other desirable yield phenotypes that can be used in the breeding programs of the present disclosure include:

High Yield Natural Light Production Long Season—Selection based on yield performance for early ripening varieties during long seasons.

High Yield Natural Light Production Short Season—Selection based on yield performance of late ripening varieties during long season and/or yield of plants that ripen in winter months and at low light levels.

High Yield Indoor Production—Selection based solely on plant yield performance in artificial lighting (e.g., HID). Another important phenotype that is important for cannabis production is structural features for easy harvesting.

Structure for Manual Trim/Market—Selections are based on the relative ratio by weight of finished flower. This usually is directly related to dense trichome morphology with very few sun leaves.

Structure for Automated Trimming—Selection based on flower morphology that is more kola (continuous long bud) with many sun leaves protruding from large inflorescences. Overall flower size is typically large, but trichomes are less densely packed and overall inflorescence is less dense than what is traditionally selected for manual trim.

Root Structure—Positive root selection is marked by overall root vigor and adventitious root growth, ease of transplant, rate of root development on clonal propagations, and root shooting from tissue culture samples. Root selections can also be based on resistance to soil and hydroponic pathogens including Pythium.

Vigor—Selection for plant vigor are marked by tremendous grow rates and robust stem/stalk infrastructure. Often times, selection display morphologies that are very much enlarged compared to sibling progeny.

Fungal Resistance—Selections based on plant that exhibit immunity or partial immunity to fungal diseases and pathogens including but not limited to powdery mildew, botrytis, downy mildew among others.

Harvesting by Combine—Selections based on plant ideotypes that are better suited for large-scale, outdoor, field production and harvesting. Examples of applicable traits include stem lodging resistance, stems suitable for machine cutting, resistance to prevalent pests in field production (e.g., corn borers), suitable height for machine combining, etc.

For a non-limiting list of cannabinoid phenotypes, please see Marijuana Botany, An Advanced study: The Propagation and Breeding of Distinctive Cannabis by Robert Connell Clarke.

The present invention also relates to variants, mutants and modifications of the seeds, plant parts and/or whole plants of the cannabis plants of the present invention. Variants, mutants and trivial modifications of the seeds, plants, plant parts, plant cells of the present invention can be generated by methods well known and available to one skilled in the art, including but not limited to, mutagenesis (e.g., chemical mutagenesis, radiation mutagenesis, transposon mutagenesis, insertional mutagenesis, signature tagged mutagenesis, site-directed mutagenesis, and natural mutagenesis), knock-outs/knock-ins, antisense and RNA interference. For more information of mutagenesis in plants, such as agents, protocols, see Acquaah et al. (Principles of plant genetics and breeding, Wiley-Blackwell, 2007, ISBN 1405136464, 9781405136464, which is herein incorporated by reference in its entity).

The present invention also relates to a mutagenized population of the cannabis plants of the present invention, and methods of using such populations. In some embodiments, the mutagenized population can be used in screening for new cannabis lines that comprise one or more or all of the morphological, physiological, biological, and/or chemical characteristics of cannabis plants of the present invention. In some embodiments, the new cannabis plants obtained from the screening process comprise one or more or all of the morphological, physiological, biological, and/or chemical characteristics of cannabis plants of the present invention, and one or more additional or different new morphological, physiological, biological, and/or chemical characteristic.

The mutagenized population of the present invention can be used in Targeting Induced Local Lesions in Genomes (TILLING) screening method, which combines a standard and efficient technique of mutagenesis with a chemical mutagen (e.g., Ethyl methanesulfonate (EMS)) with a sensitive DNA screening-technique that identifies single base mutations (also called point mutations) in a target gene. Detailed description on methods and compositions on TILLING® can be found in Till et al. (Discovery of induced point mutations in maize genes by TILLING, BMC Plant Biology 2004, 4:12), Weil et al., (TILLING in Grass Species, Plant Physiology January 2009 vol. 149 no. 1 158-164), Comai, L. and S. Henikoff (“TILLING: practical single-nucleotide mutation discovery.” Plant J 45(4): 684-94), McCallum et al., (Nature Biotechnology, 18: 455-457, 2000), McCallum et al., (Plant Physiology, 123: 439-442, 2000), Colbert et al., (Plant Physiol. 126(2): 480-484, 2001), U.S. Pat. No. 5,994,075, U.S. Patent Application Publication No. 2004/0053236A, and International Patent Application Publication Nos. WO 2005/055704 and WO 2005/048692, each of which is hereby incorporated by reference for all purposes.

### Cannabis Breeding Methods

In some embodiments, the plants of the present invention can be used to produce new plant varieties. In some embodiments, the plants are used to develop new, unique and superior varieties or hybrids with desired phenotypes.

In some embodiments, selection methods, e.g., molecular marker assisted selection, can be combined with breeding methods to accelerate the process. Additional breeding methods have been known to one of ordinary skill in the art, e.g., methods discussed in Chahal and Gosal (Principles and procedures of plant breeding: biotechnological and conventional approaches, CRC Press, 2002, ISBN 084931321X, 9780849313219), Taji et al. (In vitro plant breeding, Routledge, 2002, ISBN 156022908X, 9781560229087), Richards (Plant breeding systems, Taylor & Francis US, 1997, ISBN 0412574500, 9780412574504), Hayes (Methods of Plant Breeding, Publisher: READ BOOKS, 2007, ISBN1406737062, 9781406737066), each of which is incorporated by reference in its entirety for all purposes. Cannabis genome has been sequenced recently (van Bakel et al., The draft genome and transcriptome of Cannabis sativa, Genome Biology, 12(10):R102, 2011). Molecular makers for cannabis plants are described in Datwyler et al. (Genetic variation in hemp and marijuana (Cannabis sativa L.) according to amplified fragment length polymorphisms, J Forensic Sci. 2006 March; 51(2):371-5.), Pinarkara et al., (RAPD analysis of seized marijuana (Cannabis sativa L.) in Turkey, Electronic Journal of Biotechnology, 12(1), 2009), Hakki et al., (Inter simple sequence repeats separate efficiently hemp from marijuana (Cannabis sativa L.), Electronic Journal of Biotechnology, 10(4), 2007), Datwyler et al., (Genetic Variation in Hemp and Marijuana (Cannabis sativa L.) According to Amplified Fragment Length Polymorphisms, J Forensic Sci, March 2006, 51(2):371-375), Gilmore et al. (Isolation of microsatellite markers in Cannabis sativa L. (marijuana), Molecular Ecology Notes, 3(1):105-107, March 2003), Pacifico et al., (Genetics and marker-assisted selection of chemotype in Cannabis sativa L.), Molecular Breeding (2006) 17:257-268), and Mendoza et al., (Genetic individualization of Cannabis sativa by a short tandem repeat multiplex system, Anal Bioanal Chem (2009) 393:719-726), each of which is herein incorporated by reference in its entirety for all purposes.

In some embodiments, molecular markers are designed and made, based on the genome of the plants of the present application. In some embodiments, the molecular markers are selected from Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs). Amplified Fragment Length Polymorphisms (AFLPs), and Simple Sequence Repeats (SSRs) which are also referred to as Microsatellites, etc. Methods of developing molecular markers and their applications are described by Avise (Molecular markers, natural history, and evolution, Publisher: Sinauer Associates, 2004, ISBN 0878930418, 9780878930418), Srivastava et al. (Plant biotechnology and molecular markers, Publisher: Springer, 2004, ISBN1402019114, 9781402019111), and Vienne (Molecular markers in plant genetics and biotechnology, Publisher: Science Publishers, 2003), each of which is incorporated by reference in its entirety for all purposes.

The molecular markers can be used in molecular marker assisted breeding. For example, the molecular markers can be utilized to monitor the transfer of the genetic material. In some embodiments, the transferred genetic material is a gene of interest, such as genes that contribute to one or more favorable agronomic phenotypes when expressed in a plant cell, a plant part, or a plant.

Details of existing cannabis plants varieties and breeding methods are described in Potter et al. (2011, World Wide Weed: Global Trends in Cannabis Cultivation and Its Control), Holland (2010, The Pot Book: A Complete Guide to Cannabis, Inner Traditions/Bear & Co, ISBN1594778981, 9781594778988), Green I (2009, The Cannabis Grow Bible: The Definitive Guide to Growing Marijuana for Recreational and Medical Use, Green Candy Press, 2009, ISBN 1931160589, 9781931160582), Green II (2005, The Cannabis Breeder's Bible: The Definitive Guide to Marijuana Genetics, Cannabis Botany and Creating Strains for the Seed Market, Green Candy Press, 1931160279, 9781931160278), Starks (1990, Marijuana Chemistry: Genetics, Processing & Potency, ISBN 0914171399, 9780914171393), Clarke (1981, Marijuana Botany, an Advanced Study: The Propagation and Breeding of Distinctive Cannabis, Ronin Publishing, ISBN 091417178X, 9780914171782), Short (2004, Cultivating Exceptional Cannabis: An Expert Breeder Shares His Secrets, ISBN 1936807122, 9781936807123), Cervantes (2004, Marijuana Horticulture: The Indoor/Outdoor Medical Grower's Bible, Van Patten Publishing, ISBN 187882323X, 9781878823236), Franck et al. (1990, Marijuana Grower's Guide, Red Eye Press, ISBN 0929349016, 9780929349015), Grotenhermen and Russo (2002, Cannabis and Cannabinoids: Pharmacology, Toxicology, and Therapeutic Potential, Psychology Press, ISBN 0789015080, 9780789015082), Rosenthal (2007, The Big Book of Buds: More Marijuana Varieties from the World's Great Seed Breeders, ISBN 1936807068, 9781936807062), Clarke, RC (Cannabis: Evolution and Ethnobotany 2013 (In press)), King, J (Cannabible Vols 1-3, 2001-2006), and four volumes of Rosenthal's Big Book of Buds series (2001, 2004, 2007, and 2011), each of which is herein incorporated by reference in its entirety for all purposes.

Classical breeding methods can be included in the present invention to introduce one or more recombinant expression cassettes of the present invention into other plant varieties, or other close-related species that are compatible to be crossed with the transgenic plant of the present invention.

In some embodiments, said method comprises (i) crossing any one of the plants of the present invention comprising the expression cassette as a donor to a recipient plant line to create a F1 population, (ii) selecting offspring that have expression cassette. Optionally, the offspring can be further selected by testing the expression of the gene of interest.

In some embodiments, complete chromosomes of the donor plant are transferred. For example, the transgenic plant with the expression cassette can serve as a male or female parent in a cross pollination to produce offspring plants, wherein by receiving the transgene from the donor plant, the offspring plants have the expression cassette.

In a method for producing plants having the expression cassette, protoplast fusion can also be used for the transfer of the transgene from a donor plant to a recipient plant. Protoplast fusion is an induced or spontaneous union, such as a somatic hybridization, between two or more protoplasts (cells in which the cell walls are removed by enzymatic treatment) to produce a single bi- or multi-nucleate cell. The fused cell that may even be obtained with plant species that cannot be interbred in nature is tissue cultured into a hybrid plant exhibiting the desirable combination of traits. More specifically, a first protoplast can be obtained from a plant having the expression cassette. A second protoplast can be obtained from a second plant line, optionally from another plant species or variety, preferably from the same plant species or variety, that comprises commercially desirable characteristics, such as, but not limited to disease resistance, insect resistance, valuable grain characteristics (e.g., increased seed weight and/or seed size) etc. The protoplasts are then fused using traditional protoplast fusion procedures, which are known in the art to produce the cross.

Alternatively, embryo rescue may be employed in the transfer of the expression cassette from a donor plant to a recipient plant. Embryo rescue can be used as a procedure to isolate embryos from crosses wherein plants fail to produce viable seed. In this process, the fertilized ovary or immature seed of a plant is tissue cultured to create new plants (see Pierik, 1999, In vitro culture of higher plants, Springer, ISBN 079235267x, 9780792352679, which is incorporated herein by reference in its entirety).

In some embodiments, the recipient plant is an elite line having one or more certain desired traits. Examples of desired traits include but are not limited to those that result in increased biomass production, production of specific chemicals, increased seed production, improved plant material quality, increased seed oil content, etc. Additional examples of desired traits includes pest resistance, vigor, development time (time to harvest), enhanced nutrient content, novel growth patterns, aromas or colors, salt, heat, drought and cold tolerance, and the like. Desired traits also include selectable marker genes (e.g., genes encoding herbicide or antibiotic resistance used only to facilitate detection or selection of transformed cells), hormone biosynthesis genes leading to the production of a plant hormone (e.g., auxins, gibberellins, cytokinins, abscisic acid and ethylene that are used only for selection), or reporter genes (e.g. luciferase, -glucuronidase, chloramphenicol acetyl transferase (CAT, etc.). The recipient plant can also be a plant with preferred chemical compositions, e.g., compositions preferred for medical use or industrial applications.

Classical breeding methods can be used to produce new varieties of cannabis according to the present invention. Newly developed F1 hybrids can be reproduced via asexual reproduction.

Open-Pollinated Populations. The improvement of open-pollinated populations of such crops as rye, many maizes and sugar beets, herbage grasses, legumes such as alfalfa and clover, and tropical tree crops such as cacao, coconuts, oil palm and some rubber, depends essentially upon changing gene-frequencies towards fixation of favorable alleles while maintaining a high (but far from maximal) degree of heterozygosity. Uniformity in such populations is impossible and trueness-to-type in an open-pollinated variety is a statistical feature of the population as a whole, not a characteristic of individual plants. Thus, the heterogeneity of open-pollinated populations contrasts with the homogeneity (or virtually so) of inbred lines, clones and hybrids.

Population improvement methods fall naturally into two groups, those based on purely phenotypic selection, normally called mass selection, and those based on selection with progeny testing. Interpopulation improvement utilizes the concept of open breeding populations; allowing genes to flow from one population to another. Plants in one population (cultivar, strain, ecotype, or any germplasm source) are crossed either naturally (e.g., by wind) or by hand or by bees (commonly Apis mellifera L. or Megachile rotundata F.) with plants from other populations. Selection is applied to improve one (or sometimes both) population(s) by isolating plants with desirable traits from both sources.

There are basically two primary methods of open-pollinated population improvement. First, there is the situation in which a population is changed en masse by a chosen selection procedure. The outcome is an improved population that is indefinitely propagatable by random-mating within itself in isolation. Second, the synthetic variety attains the same end result as population improvement but is not itself propagatable as such; it has to be reconstructed from parental lines or clones. These plant breeding procedures for improving open-pollinated populations are well known to those skilled in the art and comprehensive reviews of breeding procedures routinely used for improving cross-pollinated plants are provided in numerous texts and articles, including: Allard, Principles of Plant Breeding, John Wiley & Sons, Inc. (1960); Simmonds, Principles of Crop Improvement, Longman Group Limited (1979); Hallauer and Miranda, Quantitative Genetics in Maize Breeding, Iowa State University Press (1981); and, Jensen, Plant Breeding Methodology, John Wiley & Sons, Inc. (1988).

Mass Selection. In mass selection, desirable individual plants are chosen, harvested, and the seed composited without progeny testing to produce the following generation. Since selection is based on the maternal parent only, and there is no control over pollination, mass selection amounts to a form of random mating with selection. As stated herein, the purpose of mass selection is to increase the proportion of superior genotypes in the population.

Synthetics. A synthetic variety is produced by crossing inter se a number of genotypes selected for good combining ability in all possible hybrid combinations, with subsequent maintenance of the variety by open pollination. Whether parents are (more or less inbred) seed-propagated lines, as in some sugar beet and beans (Vicia) or clones, as in herbage grasses, clovers and alfalfa, makes no difference in principle. Parents are selected on general combining ability, sometimes by test crosses or topcrosses, more generally by polycrosses. Parental seed lines may be deliberately inbred (e.g. by selfing or sib crossing). However, even if the parents are not deliberately inbred, selection within lines during line maintenance will ensure that some inbreeding occurs. Clonal parents will, of course, remain unchanged and highly heterozygous.

Whether a synthetic can go straight from the parental seed production plot to the farmer or must first undergo one or two cycles of multiplication depends on seed production and the scale of demand for seed. In practice, grasses and clovers are generally multiplied once or twice and are thus considerably removed from the original synthetic.

While mass selection is sometimes used, progeny testing is generally preferred for polycrosses, because of their operational simplicity and obvious relevance to the objective, namely exploitation of general combining ability in a synthetic.

The numbers of parental lines or clones that enter a synthetic vary widely. In practice, numbers of parental lines range from 10 to several hundred, with 100-200 being the average. Broad based synthetics formed from 100 or more clones would be expected to be more stable during seed multiplication than narrow based synthetics.

Pedigreed varieties. A pedigreed variety is a superior genotype developed from selection of individual plants out of a segregating population followed by propagation and seed increase of self-pollinated offspring and careful testing of the genotype over several generations. This is an open pollinated method that works well with naturally self-pollinating species. This method can be used in combination with mass selection in variety development. Variations in pedigree and mass selection in combination are the most common methods for generating varieties in self-pollinated crops.

Hybrids. A hybrid is an individual plant resulting from a cross between parents of differing genotypes. Commercial hybrids are now used extensively in many crops, including corn (maize), sorghum, sugar beet, sunflower and broccoli. Hybrids can be formed in a number of different ways, including by crossing two parents directly (single cross hybrids), by crossing a single cross hybrid with another parent (three-way or triple cross hybrids), or by crossing two different hybrids (four-way or double cross hybrids).

Strictly speaking, most individuals in an out breeding (i.e., open-pollinated) population are hybrids, but the term is usually reserved for cases in which the parents are individuals whose genomes are sufficiently distinct for them to be recognized as different species or subspecies. Hybrids may be fertile or sterile depending on qualitative and/or quantitative differences in the genomes of the two parents. Heterosis, or hybrid vigor, is usually associated with increased heterozygosity that results in increased vigor of growth, survival, and fertility of hybrids as compared with the parental lines that were used to form the hybrid. Maximum heterosis is usually achieved by crossing two genetically different, highly inbred lines.

### Plant Transformation

Specialty Cannabis plants of the present invention can be further modified by introducing into the plants one or more transgenes which when expressed lead to desired phenotypes. The most common method for the introduction of new genetic material into a plant genome involves the use of living cells of the bacterial pathogen Agrobacterium tumefaciens to literally inject a piece of DNA, called transfer or T-DNA, into individual plant cells (usually following wounding of the tissue) where it is targeted to the plant nucleus for chromosomal integration. There are numerous patents governing Agrobacterium mediated transformation and particular DNA delivery plasmids designed specifically for use with Agrobacterium—for example, U.S. Pat. No. 4,536,475, EP0265556, EP0270822, WO8504899, WO8603516, U.S. Pat. No. 5,591,616, EP0604662, EP0672752, WO8603776, WO9209696, WO9419930, WO9967357, U.S. Pat. No. 4,399,216, WO8303259, U.S. Pat. No. 5,731,179, EP068730, WO9516031, U.S. Pat. Nos. 5,693,512, 6,051,757 and EP904362A1. Agrobacterium-mediated plant transformation involves as a first step the placement of DNA fragments cloned on plasmids into living Agrobacterium cells, which are then subsequently used for transformation into individual plant cells. Agrobacterium-mediated plant transformation is thus an indirect plant transformation method. Methods of Agrobacterium-mediated plant transformation that involve using vectors with no T-DNA are also well known to those skilled in the art and can have applicability in the present invention. See, for example, U.S. Pat. No. 7,250,554, which utilizes P-DNA instead of T-DNA in the transformation vector.

Direct plant transformation methods using DNA have also been reported. The first of these to be reported historically is electroporation, which utilizes an electrical current applied to a solution containing plant cells (M. E. Fromm et al., Nature, 319, 791 (1986); H. Jones et al., Plant Mol. Biol., 13, 501 (1989) and H. Yang et al., Plant Cell Reports, 7, 421 (1988). Another direct method, called “biolistic bombardment”, uses ultrafine particles, usually tungsten or gold, that are coated with DNA and then sprayed onto the surface of a plant tissue with sufficient force to cause the particles to penetrate plant cells, including the thick cell wall, membrane and nuclear envelope, but without killing at least some of them (U.S. Pat. Nos. 5,204,253, 5,015,580). A third direct method uses fibrous forms of metal or ceramic consisting of sharp, porous or hollow needle-like projections that literally impale the cells, and also the nuclear envelope of cells. Both silicon carbide and aluminum borate whiskers have been used for plant transformation (Mizuno et al., 2004; Petolino et al., 2000; U.S. Pat. No. 5,302,523 US Application 20040197909) and also for bacterial and animal transformation (Kaepler et al., 1992; Raloff, 1990; Wang, 1995). There are other methods reported, and undoubtedly, additional methods will be developed. However, the efficiencies of each of these indirect or direct methods in introducing foreign DNA into plant cells are invariably extremely low, making it necessary to use some method for selection of only those cells that have been transformed, and further, allowing growth and regeneration into plants of only those cells that have been transformed.

For efficient plant transformation, a selection method must be employed such that whole plants are regenerated from a single transformed cell and every cell of the transformed plant carries the DNA of interest. These methods can employ positive selection, whereby a foreign gene is supplied to a plant cell that allows it to utilize a substrate present in the medium that it otherwise could not use, such as mannose or xylose (for example, refer U.S. Pat. Nos. 5,767,378; 5,994,629). More typically, however, negative selection is used because it is more efficient, utilizing selective agents such as herbicides or antibiotics that either kill or inhibit the growth of nontransformed plant cells and reducing the possibility of chimeras. Resistance genes that are effective against negative selective agents are provided on the introduced foreign DNA used for the plant transformation. For example, one of the most popular selective agents used is the antibiotic kanamycin, together with the resistance gene neomycin phosphotransferase (nptII), which confers resistance to kanamycin and related antibiotics (see, for example, Messing & Vierra, Gene 19: 259-268 (1982); Bevan et al., Nature 304:184-187 (1983)). However, many different antibiotics and antibiotic resistance genes can be used for transformation purposes (refer U.S. Pat. Nos. 5,034,322, 6,174,724 and 6,255,560). In addition, several herbicides and herbicide resistance genes have been used for transformation purposes, including the bar gene, which confers resistance to the herbicide phosphinothricin (White et al., Nucl Acids Res 18: 1062 (1990), Spencer et al., Theor Appl Genet 79: 625-631(1990), U.S. Pat. Nos. 4,795,855, 5,378,824 and 6,107,549). In addition, the dhfr gene, which confers resistance to the anticancer agent methotrexate, has been used for selection (Bourouis et al., EMBO J. 2(7):1099-1104 (1983).

Genes can be introduced in a site directed fashion using homologous recombination. Homologous recombination permits site specific modifications in endogenous genes and thus inherited or acquired mutations may be corrected, and/or novel alterations may be engineered into the genome. Homologous recombination and site-directed integration in plants are discussed in, for example, U.S. Pat. Nos. 5,451,513, 5,501,967 and 5,527,695.

Methods of producing transgenic plants are well known to those of ordinary skill in the art. Transgenic plants can now be produced by a variety of different transformation methods including, but not limited to, electroporation; microinjection; microprojectile bombardment, also known as particle acceleration or biolistic bombardment; viral-mediated transformation; and Agrobacterium-mediated transformation. See, for example, U.S. Pat. Nos. 5,405,765; 5,472,869; 5,538,877; 5,538,880; 5,550,318; 5,641,664; 5,736,369 and 5,736,369; and International Patent Application Publication Nos. WO/2002/038779 and WO/2009/117555; Lu et al., (Plant Cell Reports, 2008, 27:273-278); Watson et al., Recombinant DNA, Scientific American Books (1992); Hinchee et al., Bio/Tech. 6:915-922 (1988); McCabe et al., Bio/Tech. 6:923-926 (1988); Toriyama et al., Bio/Tech. 6:1072-1074 (1988); Fromm et al., BioTech. 8:833-839 (1990); Mullins et al., Bio./Tech. 8:833-839 (1990); Hiei et al., Plant Molecular Biology 35:205-218 (1997); Ishida et al., Nature Biotechnology 14:745-750 (1996); Zhang et al., Molecular Biotechnology 8:223-231 (1997); Ku et al., Nature Biotechnology 17:76-80 (1999); and, Raineri et al., Bio/Tech. 8:33-38 (1990)), each of which is expressly incorporated herein by reference in their entirety. Other references teaching the transformation of cannabis plants and the production of callus tissue include Raharjo et al 2006, “Callus Induction and Phytochemical Characterization of Cannabis sativa Cell Suspension Cultures”, Indo. J. Chem 6 (1) 70-74; and “The biotechnology of Cannabis sativa” by Sam R. Zwenger, electronically published April, 2009.

Microprojectile bombardment is also known as particle acceleration, biolistic bombardment, and the gene gun (Biolistic® Gene Gun). The gene gun is used to shoot pellets that are coated with genes (e.g., for desired traits) into plant seeds or plant tissues in order to get the plant cells to then express the new genes. The gene gun uses an actual explosive (.22 caliber blank) to propel the material. Compressed air or steam may also be used as the propellant. The Biolistic@ Gene Gun was invented in 1983-1984 at Cornell University by John Sanford, Edward Wolf, and Nelson Allen. It and its registered trademark are now owned by E. I. du Pont de Nemours and Company. Most species of plants have been transformed using this method.

Agrobacterium tumefaciens is a naturally occurring bacterium that is capable of inserting its DNA (genetic information) into plants, resulting in a type of injury to the plant known as crown gall. Most species of plants can now be transformed using this method, including cucurbitaceous species. A transgenic plant formed using Agrobacterium transformation methods typically contains a single gene on one chromosome, although multiple copies are possible. Such transgenic plants can be referred to as being hemizygous for the added gene. A more accurate name for such a plant is an independent segregant, because each transformed plant represents a unique T-DNA integration event (U.S. Pat. No. 6,156,953). A transgene locus is generally characterized by the presence and/or absence of the transgene. A heterozygous genotype in which one allele corresponds to the absence of the transgene is also designated hemizygous (U.S. Pat. No. 6,008,437).

General transformation methods, and specific methods for transforming certain plant species (e.g., maize) are described in U.S. Pat. Nos. 4,940,838, 5,464,763, 5,149,645, 5,501,967, 6,265,638, 4,693,976, 5,635,381, 5,731,179, 5,693,512, 6,162,965, 5,693,512, 5,981,840, 6,420,630, 6,919,494, 6,329,571, 6,215,051, 6,369,298, 5,169,770, 5,376,543, 5,416,011, 5,569,834, 5,824,877, 5,959,179, 5,563,055, and 5,968,830, each of which is incorporated herein by reference in its entirety for all purposes.

Non-limiting examples of methods for transforming cannabis plants and cannabis tissue culture methods are described in Zweger (The Biotechnology of Cannabis sativa, April 2009); MacKinnon (Genetic transformation of Cannabis sativa Linn: a multipurpose fiber crop, doctoral thesis, University of Dundee, Scotland, 2003), MacKinnon et al. (Progress towards transformation of fiber hemp, Scottish Crop Research, 2000), and US 20120311744, each of which is herein incorporated by reference in its entirety for all purposes. The transformation can be physical, chemical and/or biological.

In some embodiments, the present disclosure teaches the genetic modification of Specialty Cannabis. In some embodiments, the Specialty Cannabis of the present disclosure comprise one or more transgenes, or DNA edits. Thus in some embodiments, the present disclosure teaches transformation of plants (e.g., via agrobacterium, gene gun, or other delivery mechanism). In other embodiments, the present disclosure teaches gene editing with CRISPR, as disclosed in U.S. Pat. Nos. 8,697,359, 9,790,490, and U.S. application Ser. No. 15/482,603.

### Specialty Cannabinoid Compositions

In some embodiments, the present disclosure teaches cannabinoid compositions comprising high CBG contents. In some embodiments, the compositions of the present disclosure are completely derived from cannabis extractions (i.e., all components are derived from the cannabis plant). In other embodiments, the present disclosure teaches cannabinoid compositions in which only the active cannabinoid and terpene components must be derived from the cannabis plant. In yet other embodiments, the present disclosure teaches cannabinoid compositions in which one or more components are derived from sources other than the cannabis plant (e.g., from other organisms, or chemically synthesized).

For example, the cannabinoid compositions of the present disclosure can, in some embodiments, comprise cannabinoids produced via standard chemical, biochemical, or biocatalytic methods. Persons having skill in the art will be familiar with various synthesis methods, including those of U.S. Pat. No. 9,359,625 and Taura et al. 1996, The Journal of Biological Chemistry, Vol. 271, No. 21, p. 17411-17416.

In some embodiments, the compositions of the present disclosure mimic the cannabinoid and Terpene Profiles of the Specialty Cannabis plants disclosed herein. That is, in some embodiments, the cannabinoid compositions comprise CBG/CBGV with non-trace levels of at least one other non-CBG cannabinoid. Extractions methods designed to preserve cannabinoid and Terpene Profiles are disclosed in other sections of this application.

In some embodiments, the present disclosure teaches methods of supplementing cannabis extracts with one or more cannabinoid or terpene to account for any losses of the compounds during the extraction process. In yet other embodiments, the present disclosure teaches the formulation of cannabis compositions from individual components (i.e., by mixing individual cannabinoid and terpene components obtained from the same or different sources).

In some embodiments, the cannabinoid compositions of the present disclosure are designed to mimic the organoleptic experience produced by the Specialty Cannabis.

In some embodiments, the cannabinoid compositions of the present disclosure comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% cannabinoids by weight based on the weight of the cannabinoid composition, and all ranges therebetween. Thus, in some embodiments, the cannabinoid compositions of the present disclosure comprise 1-60%, 1-80%, 1-25% cannabinoid content by weight.

In some embodiments, the cannabinoid compositions of the present disclosure comprise more than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% cannabinoids by weight based on the weight of the cannabinoid composition.

In some embodiments, the cannabinoid compositions of the present disclosure comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% CBG by weight based on the weight of the cannabinoid composition, and all ranges therebetween.

In some embodiments, the cannabinoid compositions of the present disclosure comprise more than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% CBG by weight based on the weight of the cannabinoid composition.

In some embodiments, the cannabinoid compositions of the present disclosure comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% CBGV by weight based on the weight of the cannabinoid composition, and all ranges therebetween.

In some embodiments, the cannabinoid compositions of the present disclosure comprise more than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% CBGV by weight based on the weight of the cannabinoid composition.

In some embodiments, the cannabinoid compositions of the present disclosure comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% THC by weight based on the weight of the cannabinoid composition, and all ranges therebetween.

In some embodiments, the cannabinoid compositions of the present disclosure comprise more than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% THC by weight based on the weight of the cannabinoid composition.

In some embodiments, the cannabinoid compositions of the present disclosure comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% THC by weight based on the weight of the cannabinoid composition, and all ranges therebetween, together with 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, or 93% of one or more primary cannabinoid by weight based on the weight of the cannabinoid composition, and all ranges therebetween.

In some embodiments, the cannabinoid compositions of the present disclosure comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% CBD by weight based on the weight of the cannabinoid composition, and all ranges therebetween.

In some embodiments, the cannabinoid compositions of the present disclosure comprise more than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% CBD by weight based on the weight of the cannabinoid composition.

In some embodiments, the cannabinoid compositions of the present disclosure comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% CBD by weight based on the weight of the cannabinoid composition, and all ranges therebetween, together with 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49/c, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, or 93% of one or more primary cannabinoid by weight based on the weight of the cannabinoid composition, and all ranges therebetween.

In some embodiments, the cannabinoid compositions of the present disclosure comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 5r/, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% propyl cannabinoids by weight based on the weight of the cannabinoid composition, and all ranges therebetween.

In some embodiments, the cannabinoid compositions of the present disclosure comprise more than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% propyl cannabinoids by weight based on the weight of the cannabinoid composition.

In some embodiments, the cannabinoid compositions of the present disclosure comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% propyl cannabinoids by weight based on the weight of the cannabinoid composition, and all ranges therebetween, together with 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, or 93% of one or more primary cannabinoid by weight based on the weight of the cannabinoid composition, and all ranges therebetween.

In some embodiments, the cannabinoid compositions of the present disclosure comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% CBC by weight based on the weight of the cannabinoid composition, and all ranges therebetween.

In some embodiments, the cannabinoid compositions of the present disclosure comprise more than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% CBC by weight based on the weight of the cannabinoid composition.

In some embodiments, the cannabinoid compositions of the present disclosure comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% CBC by weight based on the weight of the cannabinoid composition, and all ranges therebetween, together with 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 860, 87%, 88%, 89%, 90%, 91%, 92%, or 93% of one or more primary cannabinoid by weight based on the weight of the cannabinoid composition, and all ranges therebetween.

In some embodiments, the cannabinoid compositions of the present disclosure comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 5r/, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% THCV by weight based on the weight of the cannabinoid composition, and all ranges therebetween.

In some embodiments, the cannabinoid compositions of the present disclosure comprise more than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% THCV by weight based on the weight of the cannabinoid composition.

In some embodiments, the cannabinoid compositions of the present disclosure comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% THCV by weight based on the weight of the cannabinoid composition, and all ranges therebetween, together with 1%, 2%, 3%, 4%, 5%, 6%, 7%, 98%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, or 93% of one or more primary cannabinoid by weight based on the weight of the cannabinoid composition, and all ranges therebetween.

In some embodiments, the cannabinoid compositions of the present disclosure comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% CBDV by weight based on the weight of the cannabinoid composition, and all ranges therebetween.

In some embodiments, the cannabinoid compositions of the present disclosure comprise more than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% CBDV by weight based on the weight of the cannabinoid composition.

In some embodiments, the cannabinoid compositions of the present disclosure comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39/c, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% CBDV by weight based on the weight of the cannabinoid composition, and all ranges therebetween, together with 1%, 2%, 3%, 4%, 5%, 6%, 7%, 8%, 9%, 10%, 11%, 12%, 13%, 14%, 15%, 16%, 17%, 18%, 19/, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, or 93% of one or more primary cannabinoid by weight based on the weight of the cannabinoid composition, and all ranges therebetween.

In some embodiments, the cannabinoid compositions of the present disclosure comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% CBCV by weight based on the weight of the cannabinoid composition, and all ranges therebetween.

In some embodiments, the cannabinoid compositions of the present disclosure comprise more than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%0, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% CBCV by weight based on the weight of the cannabinoid composition.

In some embodiments, the cannabinoid compositions of the present disclosure comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% CBCV by weight based on the weight of the cannabinoid composition, and all ranges therebetween, together with 1%, 2%, 3%, 4%, 5%, 6%, 7%, 98%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%¹, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, or 93% of one or more primary cannabinoid by weight based on the weight of the cannabinoid composition, and all ranges therebetween.

In some embodiments, the cannabinoid compositions of the present disclosure comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% NGCs by weight based on the weight of the cannabinoid composition, and all ranges therebetween.

In some embodiments, the cannabinoid compositions of the present disclosure comprise more than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90°, 91%, 92%, 93%, 94%, 95%, 96%, 97%, 98%, or 99% NGCs by weight based on the weight of the cannabinoid composition.

In some embodiments, the cannabinoid compositions of the present disclosure comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39/c, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% NGCs by weight based on the weight of the cannabinoid composition, and all ranges therebetween, together with 1%, 2%, 3%, 4%, 5%, 6%, 7%, 98%, 9%, 10%, 11%, 12%, 13%, 14%, 15%, 16%, 17%, 18%, 19/, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%¹, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, or 93% of one or more primary cannabinoid by weight based on the weight of the cannabinoid composition, and all ranges therebetween.

In some embodiments, the cannabinoid compositions of the present disclosure produce comprise organoleptically pleasing Terpene Profiles.

In some embodiments, the cannabinoid compositions of the present invention has an absolute content of any one of the 17 terpenes in the Terpene Profile that is about 0%, 0.01%, 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66/, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99%, 1%, 1.01%, 1.02%, 1.03%, 1.04%, 1.05%, 1.06%, 1.07%, 1.08%, 1.09%, 1.1%, 1.11%, 1.12%, 1.13%, 1.14%, 1.15%, 1.16%, 1.17%, 1.18%, 1.19%, 1.2%, 1.21%, 1.22%, 1.23%, 1.24%, 1.25%, 1.26%, 1.27%, 1.28%, 1.29%, 1.3%, 1.31%, 1.32%, 1.33%, 1.34%, 1.35%, 1.36%, 1.37%, 1.38%, 1.39%, 1.4%, 1.41%, 1.42%, 1.43%, 1.44%, 1.45%, 1.46%, 1.47%, 1.48%, 1.49%, 1.5%, 1.51%, 1.52%, 1.53%, 1.54%, 1.55%, 1.56%, 1.57%, 1.58%, 1.59%, 1.6%, 1.61%, 1.62%, 1.63%, 1.64%, 1.65%, 1.66%, 1.67%, 1.68%, 1.69%, 1.7%, 1.71%, 1.72%, 1.73%, 1.74%, 1.75%, 1.76%, 1.77%, 1.78%, 1.79%, 1.8%, 1.81%, 1.82%, 1.83%, 1.84%, 1.85%, 1.86%, 1.87%, 1.88%, 1.89%, 1.9%, 1.91%, 1.92%, 1.93%, 1.94%, 1.95%, 1.96%, 1.97%, 1.98%, 1.99%, 2%, 2.01%, 2.02%, 2.03%, 2.04%, 2.05%, 2.06%, 2.07%, 2.08%, 2.09%, 2.1%, 2.11%, 2.12%, 2.13%, 2.14%, 2.15%, 2.16%, 2.17%, 2.18%, 2.19%, 2.2%, 2.21%, 2.22%, 2.23%, 2.24%, 2.25%, 2.26%, 2.27%, 2.28%, 2.29%, 2.3%, 2.31%, 2.32%, 2.33%, 2.34%, 2.35%, 2.36%, 2.37%, 2.38%, 2.39%, 2.4%, 2.41%, 2.42%, 2.43%, 2.44%, 2.45%, 2.46%, 2.47%, 2.48%, 2.49%, 2.5%, 2.51%, 2.52%, 2.53%, 2.54%, 2.55%, 2.56%, 2.57%, 2.58%, 2.59%, 2.6%, 2.61%, 2.62%, 2.63%, 2.64%, 2.65%, 2.66%, 2.67%, 2.68%, 2.69%, 2.7%, 2.71%, 2.72%, 2.73%, 2.74%, 2.75%, 2.76%, 2.77%, 2.78%, 2.79%, 2.8%, 2.81%, 2.82%, 2.83%, 2.84%, 2.85%, 2.86%, 2.87%, 2.88%, 2.89%, 2.9%, 2.91%, 2.92%, 2.93%, 2.94%, 2.95%, 2.96%, 2.97%, 2.98%, 2.99%, 3%, 3.2%, 3.4%, 3.6%, 3.8%, 4%, 4.2%, 4.3%, 4.4%, 4.6%, 4.8%, 5%, 5.2%, 5.4%, 5.6%, 5.8%, 6%, 6.2%, 6.4%, 6.6%, 6.8%, 7%, 7.2%, 7.4%, 7.6%, 7.8%, 8%, 9, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 660, 670, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 900, 91%, 92%, 93%, 94%, 95%, 96%, 97%, 98%, or 99% by weight of the cannabinoid composition and all ranges therebetween. Thus in some embodiments the absolute content of any one of the terpenes is between about 0.05% and about 5%. This paragraph is intended to be read as applying to any specific terpene(s) in a Terpene Profile, such that the name of any one or two or more of these terpenes as specifically referred to elsewhere herein (e.g., linalool) can replace the phrase “any one of the 17 terpenes in the Terpene Profile.”

In some embodiments, the cannabinoid compositions of the present invention has an absolute content of any one of the 17 terpenes in the Terpene Profile that is more than about 0%0, 0.01%, 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99%, 1%, 1.01%, 1.02%, 1.03%, 1.04%, 1.05%, 1.06%, 1.07%, 1.08%, 1.09%, 1.1%, 1.11%, 1.12%, 1.13%, 1.14%, 1.15%, 1.16%, 1.17%, 1.18%, 1.19%, 1.2%, 1.21%, 1.22%, 1.23%, 1.24%, 1.25%, 1.26%, 1.27%, 1.28%, 1.29%, 1.3%, 1.31%, 1.32%, 1.33%, 1.34%, 1.35%, 1.36%, 1.37%, 1.38%, 1.39%, 1.4%, 1.41%, 1.42%, 1.43%, 1.44%, 1.45%, 1.46%, 1.47%, 1.48%, 1.49%, 1.5%, 1.51%, 1.52%, 1.53%, 1.54%, 1.55%, 1.56%, 1.57%, 1.58%, 1.59%, 1.6%, 1.61%, 1.62%, 1.63%, 1.64%, 1.65%, 1.66%, 1.67%, 1.68%, 1.69%, 1.7%, 1.71%, 1.72%, 1.73%, 1.74%, 1.75%, 1.76%, 1.77%, 1.78%, 1.79%, 1.8%, 1.81%, 1.82%, 1.83%, 1.84%, 1.85%, 1.86%, 1.87%, 1.88%, 1.89%, 1.9%, 1.91%, 1.92%, 1.93%, 1.94%, 1.95%, 1.96%, 1.97%, 1.98%, 1.99%, 2%, 2.01%, 2.02%, 2.03%, 2.04%, 2.05%, 2.06%, 2.07%, 2.08%, 2.09%, 2.1%, 2.11%, 2.12%, 2.13%, 2.14%, 2.15%, 2.16%, 2.17%, 2.18%, 2.19%, 2.2%, 2.21%, 2.22%, 2.23%, 2.24%, 2.25%, 2.26%, 2.27%, 2.28%, 2.29%, 2.3%, 2.31%, 2.32%, 2.33%, 2.34%, 2.35%, 2.36%, 2.37%, 2.38%, 2.39%, 2.4%, 2.41%, 2.42%, 2.43%, 2.44%, 2.45%, 2.46%, 2.47%, 2.48%, 2.49%, 2.5%, 2.51%, 2.52%, 2.53%, 2.54%, 2.55%, 2.56%, 2.57%, 2.58%, 2.59%, 2.6%, 2.61%, 2.62%, 2.63%, 2.64%, 2.65%, 2.66%, 2.67%, 2.68%, 2.69%, 2.7%, 2.71%, 2.72%, 2.73%, 2.74%, 2.75%, 2.76%, 2.77%, 2.78%, 2.79%, 2.8%, 2.81%, 2.82%, 2.83%, 2.84%, 2.85%, 2.86%, 2.87%, 2.88%, 2.89%, 2.9%, 2.91%, 2.92%, 2.93%, 2.94%, 2.95%, 2.96%, 2.97%, 2.98%, 2.99%/, 3%, 3.2%, 3.4%, 3.6%, 3.8%, 4%, 4.2%, 4.3%, 4.4%, 4.6%, 4.8%, 5%, 5.2%, 5.4%, 5.6%, 5.8%, 6%, 6.2%, 6.4%, 6.6%, 6.8%, 7%, 7.2%, 7.4%, 7.6%, 7.8%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60% 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight of the cannabinoid composition and all ranges therebetween. This paragraph is intended to be read as applying to any specific terpene(s) in a Terpene Profile, such that the name of any one or two or more of these terpenes as specifically referred to elsewhere herein (e.g., linalool) can replace the phrase “any one of the 17 terpenes in the Terpene Profile.”

In some embodiments, the present invention teaches cannabinoid compositions with high terpene essential oil contents. In some embodiments, the cannabinoid compositions of the present invention comprise about 1%, 2%, 3%, 4%, 5%, 6%, 7%, 8%, 9%, 10%, 11%, 12%, 13%, 14%, 15%, 16%, 17%, 18%, 19%, 20%, 210%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, or 71% terpene essential oil content by weight of the cannabinoid composition, including all ranges therebetween. Thus in some embodiments the essential oil content of the cannabinoid compositions of the present disclosure are between about 0.5% and about 10% by weight. In other embodiments the essential oil contents of the cannabinoid compositions of the present invention is between about 1.0% and about 30% by weight.

In some embodiments, the present invention teaches cannabinoid compositions with high terpene essential oil contents. In some embodiments, the cannabinoid compositions of the present invention comprise more than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, or 71% terpene essential oil content by weight of the cannabinoid composition.

In some embodiments the terpene content of the cannabinoid compositions of the present disclosure are described in relative terms as a percentage composition of the total Terpene Profile. Thus for example a cannabinoid composition with 1.2% absolute terpinolene content and 1.2% limonene content and no other terpenes in the Terpene Profile would said to have 50% terpinolene and 50% limonene relative content. In some embodiments, the cannabinoid compositions of the present disclosure comprise a relative content of any one of the 17 terpenes in the Terpene Profile that is greater than or less than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 79%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, including any ranges therebetween. Thus in some embodiments the relative content of any one of the terpenes is between 0% and 100%.

In some embodiments, additional components are optionally added to the cannabinoid compositions of the present disclosure to improve the taste and/or physical properties of the composition (such as stability, viscosity, appearance of smoke as it is inhaled, etc.). Such additional components include, but are not limited to, sweeteners, natural flavorants, artificial flavorants, colorants, antioxidants, preservatives, chelating agents, viscomodulators, tonicifiers, odorants, opacifiers, suspending agents, binders, thickeners, carriers and mixtures thereof, including, but not limited to, xanthum gum, carboxymethylcellulose, carboxyethylcellulose, hydroxypropylcellulose, methylcellulose, microcrystalline cellulose, starches, dextrins, maltodextrins, other polyols (including sugar alcohols, such as sorbitol, lactitol or mannitol), carbohydrates (e.g., lactose), propylene glycol alginate, gellan gum, guar, pectin, tragacanth gum, gum acacia, locust bean gum, gum arabic, mannitol, sucralose, silicon dioxide, stearic acid, hydroxypropyl methylcellulose, mono-, di- and triglycerides (acyl glycerols), ether and sugar acetates or other acid esters such as dimethyl acetate, ethyl acetate, isopropyl acetate, ethylhexyl acetate, butyl acetate, triethyl citrate, dimethyl butyrate and the like.

In some embodiments, the cannabinoid compositions of the present disclosure comprise one or more medium chain length triglycerides (MCTs). MCTs are triglycerides whose fatty acids have an aliphatic tail of 6-12 carbon atoms. In certain embodiments, the MCT is one or more of caproic acid, caprylic acid, capric acid, lauric acid and mixtures thereof. Suitable sources of MCTs are known to those skilled in the art and include, for example, coconut oil and palm kernel oil.

In some embodiments, the cannabinoid compositions of the present disclosure comprise one or more polyesterdiols. The polyesterdiol may be a linear two to ten units polymer (also referred to as (ester)2-10 glycol), that is derived from natural or non-natural sources such as vegetables, fruits, bacteria, yeast, algae, or manufactured by chemical processes.

For example, in some embodiments, the polyesterdiol is 1) a polypropylene glycol such as: dipropylene glycol; tripropylene glycol, including tetra-, penta-, hexa-, hepta-, octa-, nona- and decapropylene glycol and other derivatives thereof; 2) a polybutylene glycol such as: dibutylene glycol, tributylene glycol, including tetra-, penta-, hexa-, hepta-, octa-, nona- and deabutylene glycol, and other derivatives thereof; 3) also including 2-10 unit polymers of rare organic ester types such as pentylene, octylene, terpentylene, nonylene, linalylene, isoamylene, isobutylene, geranylene, bornylene, benzylene and allylene, caprylylene, such as, for example, polyisobutylene glycol such as diisobutylene glycol; and 4) triethylene glycol, including tetra-, penta-, hexa-, hepta-, octa-, nona- and decaethylene glycols and other derivatives thereof such as acid or sugar conjugates, and esters or ether or alcohol derivatives.

In some embodiments, the cannabinoid compositions of the present disclosure comprise a linear polyesterdiol selected from the group consisting of: (ethylene)3-10 glycol; (propylene)2-10 glycol; (butylene)2-10 glycol; (pentylene)2-10 glycol; (octylene)2-10 glycol; (terpentylene)2-10 glycol; (nonylene)2-10 glycol; (linalylene)2-10 glycol; (isoamylene)2-10 glycol; (isobutylene)2-10 glycol; (geranylene)2-10 glycol; (bornylene)2-10 glycol; (benzylene)2-10 glycol; (allylene)2-10 glycol; and (caprylylene)2-10 glycol; acid or sugar conjugates thereof, and ester or ether or alcohol derivatives thereof.

In some embodiments, the cannabinoid compositions of the present disclosure comprises a carrier selected from the group consisting of: triethylene glycol; tetraethyleneglycol, pentaethylenglycol, hexaethyleneglycol, heptaethyleneglycol, octaethyleneglycol, nonaethylenglycol; decaethylene glycol; dipropylene glycol; tripropylene glycol; tetrapropylene glycol, pentapropylene glycol, hexapropylene glycol, heptapropylene glycol, octapropylene glycol, nonapropylene glycol; decapropylene glycol; dibutylene glycol, tributylene glycol; tetrabutylene glycol, pentabutylene glycol, hexabutylene glycol, heptabutylene glycol, octabutylene glycol, nonabutylene glycol, decabutylene glycol and diisobutylene glycol.

In some embodiments, the carrier is selected from the group consisting of borneol, camphor, 1,8-Cineole, citral, geraniol, indomethacin, limonene, linalool, linalyl acetate, p-myrcene, myrcenol, 1-menthol, menthone, neomenthol, nerol, nerolidol, a-pinene, peppermint oil, pulegone, phytol, terpineol, terpinen-4-ol, thymohydroquinone, thymol, and thymoquinone

In some embodiments, the compositions of the present disclosure comprise one or more of propylene glycol, glycerine, vegetable glycerine, and/or water.

### Cannabis Extracts

In some embodiments, the present disclosure provides for extracts from Specialty Cannabis plants. Cannabis extracts or products or the present disclosure include:

Kief—refers to trichomes collected from cannabis. The trichomes of cannabis are the areas of cannabinoid and terpene accumulation. Kief can be gathered from containers where cannabis flowers have been handled. It can be obtained from mechanical separation of the trichomes from inflorescence tissue through methods such as grinding flowers, or collecting and sifting through dust after manicuring or handling cannabis. Kief can be pressed into hashish for convenience or storage.

Hash—sometimes known as hashish, is often composed of preparations of cannabis trichomes. Hash pressed from kief is often solid.

Bubble Hash—sometimes called bubble melt hash can take on paste-like properties with varying hardness and pliability. Bubble hash is usually made via water separation in which cannabis material is placed in a cold water bath and stirred for a long time (around 1 hour). Once the mixture settles it can be sifted to collect the hash.

Solvent reduced oils—also sometimes known as hash oil, honey oil, or full melt hash among other names. This type of cannabis oil is made by soaking plant material in a chemical solvent. After separating plant material, the solvent can be boiled or evaporated off, leaving the oil behind. Butane Hash Oil is produced by passing butane over cannabis and then letting the butane evaporate. Budder or Wax is produced through isopropyl extraction of cannabis. The resulting substance is a wax like golden brown paste. Another common extraction solvent for creating cannabis oil is CO2. Persons having skill in the art will be familiar with CO2 extraction techniques and devices, including those disclosed in US 20160279183, US 2015/01505455, U.S. Pat. No. 9,730,911, and US 2018/0000857. Other guidance on CO2 extractions of cannabinoids and terpenes can be found in Perrotin-Brunel et al. “Solubility of non-psychoactive cannabinoids in supercritical carbon dioxide and comparison with psychoactive cannabinoids” The Journal of Supercritical Fluids, 55(2010) 603-608; Rovetto and Aieta, “Supercritical carbon dioxide extraction of cannabinoids from Cannabis sativa L.” The Journal of Supercritical Fluids 129 (2017) 16-27; Porto and Natolino. “Separation of aroma compounds from industrial hemp inflorescences (Cannabis sativa L.) by supercritical CO2 extraction and on-line fractionation” Industrial Crops and Products 58 (2014) 99-103; U.S. Pat. Nos. 6,403,126; 7,700,368; and US20030050334.

Tinctures—are alcoholic extracts of cannabis. These are usually made by mixing cannabis material with high proof ethanol and separating out plant material.

E-juice—are cannabis extracts dissolved in either propylene glycol, vegetable glycerin, or a combination of both. Some E-juice formulations will also include polyethylene glycol and flavorings. E-juice tends to be less viscous than solvent reduced oils and is commonly consumed on e-cigarettes or pen vaporizers.

Rick Simpson Oil (ethanol extractions)—are extracts produced by contacting cannabis with ethanol and later evaporating the vast majority of ethanol away to create a cannabinoid paste. In some embodiments, the extract produced from contacting the cannabis with ethanol is heated so as to decarboxylate the extract.

While these types of extracts have become a popular form of consuming cannabis, the extraction methods often lead to material with little or no Terpene Profile. That is, the harvest, storage, handling, and extraction methods produce an extract that is rich in cannabinoids, but often devoid of terpenes.

In some embodiments, the Specialty Cannabis of the present invention is extracted via methods that preserve the cannabinoid and terpenes. In other embodiments, said methods can be used with any cannabis plants. The extracts of the present invention are designed to produce products for human or animal consumption via inhalation (via combustion, vaporization and nebulization), buccal absorption within the mouth, oral administration, and topical application delivery methods. The present invention teaches an optimized method at which we extract compounds of interest, by extracting at the point when the drying harvested plant has reached 15% water weight, which minimizes the loss of terpenes and plant volatiles of interest. Stems are typically still ‘cool’ and ‘rubbery’ from evaporation taking place. This timeframe (or if frozen at this point in process) allow extractor to minimize terpene loss to evaporation. There is a direct correlation between cool/slow/dry and preservation of essential oils. Thus, there is a direct correlation to EO loss in flowers that dry too fast, or too hot conditions or simply dry out too much (<10% H2O).

The chemical extraction of Specialty Cannabis can be accomplished employing polar and non-polar solvents in various phases at varying pressures and temperatures to selectively or comprehensively extract terpenes, cannabinoids and other compounds of flavor, fragrance or pharmacological value for use individually or combination in the formulation of our products. The extractions can be shaped and formed into single or multiple dose packages, e.g., dabs, pellets and loads. The solvents employed for selective extraction of our cultivars may include water, carbon dioxide, 1,1,1,2-tetrafluoroethane, butane, propane, ethanol, isopropyl alcohol, hexane, and limonene, in combination or series. We can also extract compounds of interest mechanically by sieving the plant parts that produce those compounds. Measuring the plant part, i.e. trichome gland head, to be sieved via optical or electron microscopy can aid the selection of the optimal sieve pore size, ranging from 30 to 130 microns, to capture the plant part of interest. The chemical and mechanical extraction methods of the present invention can be used to produce products that combine chemical extractions with plant parts containing compounds of interest. The extracts of the present invention may also be combined with pure compounds of interest to the extractions, e.g. cannabinoids or terpenes to further enhance or modify the resulting formulation's fragrance, flavor or pharmacology.

In some embodiments, the extractions are supplemented with terpenes or cannabinoids to adjust for any loss of those compounds during extraction processes. In some embodiments, the cannabis extracts of the present invention mimic the chemistry of the cannabis flower material. In some embodiments, the cannabis extracts of the present invention will about the same cannabinoid and Terpene Profile of the dried flowers of the Specialty Cannabis of the present invention.

Extracts of the present invention can be used for vaporization, production of e-juice or tincture for e-cigarettes, or for the production of other consumable products such as edibles or topical spreads.

### Use of Specialty Cannabis and Cannabinoid Compositions in Edibles

Cannabis edibles such as candy, brownies, and other foods are a popular method of consuming cannabis for medicinal and recreational purposes. In some embodiments, the Specialty Cannabis of the present disclosure and/or the cannabinoid compositions of the present disclosure can be used to make cannabis edibles. Most cannabis edible recipes begin with the extraction of cannabinoids and terpenes, which are then used as an ingredient in various edible recipes.

In one embodiment, the cannabis extract used to make edibles out of the Specialty Cannabis of the present invention is cannabis butter. Cannabis butter is made by melting butter in a container with cannabis and letting it simmer for about half an hour, or until the butter turns green. The butter is then chilled and used in normal recipes. Other extraction methods for edibles include extraction into cooking oil, milk, cream, flour (grinding cannabis and blending with flour for baking). Lipid rich extraction mediums/edibles are believed to facilitate absorption of cannabinoids into the blood stream. THC absorbed by the body is converted by the liver into 11-hydroxy-THC. This modification increases the ability of the THC molecule to bind to the CB1 receptor and also facilitates crossing of the brain blood barrier thereby increasing the potency and duration of its effects. For additional information on various edibles that can be produced with the Specialty Cannabis of the present invention, please see (Sarah Conrique “The Vegan Stoner Cookbook: 100 easy Vegan Recipes to Much” ISBN 1607744643; “Official High Times Cannabis Cookbook” ASIN B00HB7YI8U; Bliss Cameron “Marijuana Cooking: Good Medicine Made Easy” ISBN 1931160325; Tim Pilcher “The Cannabis Cookbook: Over 35 Tasty Recipes for Meals, Munchies, and More” ISBN 0762430907).

Thus, in some embodiments, the present disclosure teaches edibles produced from the Specialty Cannabis and/or cannabinoid compositions disclosed herein.

This invention is further illustrated by the following examples, which should not be construed as limiting. The contents of all references, patents and published patent applications cited throughout this application, as well as the Figures and the Sequence Listing, are incorporated herein by reference.

### EXAMPLES

### Example 1. Chemical Analysis of Cannabinoids and Terpenes

Chemical analyses of the parental and progeny Specialty Cannabis varieties of the present disclosure, and of the cannabinoid compositions of the present disclosure, were each carried out using standard chemical separation and detection techniques well known to those skilled in the arts. Qualitative identification of cannabinoids and terpenes was carried out by GCMS, while quantitative analysis was done by GC-FID and/or HPLC-PDA (Photo Diode Array). Initial field analyses of cannabinoids was performed using thin layer chromatography as described in (“Cannabis Inflorescence & Leaf QC” from The American Herbal Pharmacopeia 2013). The in-house assays for cannabinoids included orthogonal methods of GC-FID and HPLC for the highest level of accuracy.

Plant inflorescence samples were prepared by grinding ˜5 g of dried cannabis flower material in a coffee grinder. From this homogenized material, 1000±20 mg was placed in a 50 mL falcon tube with ˜1 g of 2 mm beads and 15 mL of working solution. Each sample was placed in the bead beater (1600 MiniG from Spex Sample Prep) and homogenized on high for 6 minutes. Then approximately 2 mL of each sample were transferred to 2 mL centrifuge vials and centrifuged at 10000 g for 5 minutes. For samples suspected of having higher or lower concentrations of analytes the mass to volume ratio of the extraction could be adjusted. The neat sample was placed in a GC vial for terpene analysis without dilution. The supernatant was also diluted with working solution for GC and HPLC analysis. A 1:96 dilution provided the appropriate concentration for analysis of cannabinoids present at concentrations above 2.3%, while a 1:6 dilution allowed for analysis of cannabinoids below this level.

i. Terpenoids by Gas Chromatography-Flame Ionization Detector (GC-FID)

Terpenes were quantified by a method developed on a GC-FID instrument from Perkin Elmer (Waltham, Mass.). It is recognized among analytical scientists that terpene measurements conducted via HPLC are unreliable, as HPLC is not effective at measuring volatiles, such as terpenes. This method separates and quantifies 17 different terpenoids commonly found in cannabis plant tissue. The terpenoids are each quantified by their own individual calibration curves generated with analytical reference standards (Sigma Aldrich) and all use n-nonane as the internal standard.

The instrumentation includes a Clarus 680 gas chromatograph (GC) equipped with an autosampler, an Elite-5 column (Perkin Elmer (Waltham, Mass.), 30 m length, 0.25 mm internal diameter, 0.25 μm thickness film diameter) and a flame ionization detector (FID). Instrument control and data acquisition and analyses was accomplished by TotalChrom software version 1.2.3.4 (Perkin Elmer, Waltham, Mass.).

Calibration curves were generated by injecting each standard in triplicate and the RSDs provided the measure of precision while the absolute accuracy was determined by comparing the concentrations of the standards predicted by the calibration curve to their “known” values determined by dilution ratios. AOAC International standards for accuracy and precision were used as quality guidelines for every calibration. Check standards were run at the start, middle, and end of every analysis, and recalibration was performed when they varied more than +/−5% of their initial average response. Levels that failed the acceptance criteria and analytes were not quantified at those levels until recalibration of the instrument corrected the deficiency. Most of the curves were linear to nearly two orders of magnitude and based on the sample mass extracted (500 mg) and the two possible extraction volumes (3×3 mL or 3×5 mL), this provided quantitation of terpene levels from 0.01-0.9% or 0.02-1.5% (typical) in the plant matrix.

ii. Cannabinoids by GC-FID

Cannabinoids were quantified by an analytical method developed and run on a Perkin Elmer (Waltham, Mass.) GC-FID instrument as well. This method was developed to separate six neutral cannabinoids, CBD, CBG, CBN, THC, Δ8-THC, and CBC. The cannabinoids are each quantified by their own individual calibration curves generated with analytical reference standards (Restek) and all use tricosane as the internal standard. The retention time of THCV was determined by analyzing THV01 (vide infra) by GCMS, however since analytical standards were not available it was “quantified” by referencing the calibration curve for THC.

There was no need to consider chromatographic separation of acidic forms of the cannabinoids due to their immediate conversion to neutral form in the heated injector of the instrument, although a thorough study of the conversion efficiency of THCA was performed and is discussed in section iv. (orthogonal analyses of all samples).

The instrumentation includes a Clarus 680 gas chromatograph (GC) equipped with an autosampler, an Elite-1 column (Perkin Elmer (Waltham, Mass.), 30 m length, 0.25 mm internal diameter, 0.25 μm thickness film diameter) and a flame ionization detector (FID). Instrument control and data acquisition and analyses was accomplished by TotalChrom software version 1.2.3.4 (Perkin Elmer, Waltham, Mass.).

Calibration curves were generated by injecting each standard in triplicate and the RSDs provided the measure of precision while the absolute accuracy was determined by comparing the concentrations of the standards predicted by the calibration curve to their “known” values determined by dilution ratios. AOAC International standards for accuracy and precision were used as quality guidelines for every calibration. Check standards were run at the start, middle, and end of every analysis, and recalibration was performed when they varied more than +/−5% of their initial average response. Levels that failed the acceptance criteria and analytes were not quantified at those levels until recalibration of the instrument corrected the deficiency.

Due to the very linear nature of the FID detector, the GC-FID cannabinoid assay generally provided satisfactory results over nearly two orders of magnitude (up to 1.0 mg/mL), however in order to use the same calibration solutions and “validation” procedures for both GC and HPLC the range was reduced to that of the HPLC method. Based on the sample mass extracted (500 mg) and a 3×3 mL extraction (low oil samples), a 1:3 dilution provided quantitation of cannabinoid levels from 0.09-1.35% and the 1:40 dilution from 1.15-18% in the plant matrix. A 3×5 mL extraction (high oil samples, typical), a 1:3 dilution provided quantitation of cannabinoid levels from 0.14-2.25% and the 1:40 dilution from 1.9-30% in the plant matrix.

iii. Cannabinoids by High Performance Liquid Chromatography—Photo Diode Array Detector (HPLC-PDA)

An HPLC-PDA (also known as HPLC-DAD, or simply HPLC) assay was developed as an orthogonal method to GC-FID for cannabinoid analyses. This method quantifies six neutral cannabinoids (CBD, CBG, CBN, THC, Δ8-THC, and CBC) as well as the acid cannabinoids THCA, CBDA and CBGA amongst other acidic cannabinoids, based on calibration curves generated with analytical standards and an internal reference standard (ibuprofen).

All HPLC analyses were performed using a Agilent 1290 System (Agilent Technologies, Santa Clara, Calif.). The HPLC system comprised G4212A diode array detector, a G1316C temperature controlled column compartment, a G4226A autosampler, and a G4204A quaternary pump. Separation of the cannabinoids was achieved on a Poroshell 120 EC-C18 column (2.7μ, 150 mm×2.1 mm i.d., PN 693775-902) with a Poroshell 120 EC-C18 guard column (2.7μ, 5 mm×2.1 mm i.d., PN 821725-911) in place (Agilent Technologies, Santa Clara, Calif.). Instrument control, data acquisition and integration was achieved with OpenLab CDS ChemStation Rev C.01.06 software (Agilent Technologies, Santa Clara, Calif.).

Calibration was achieved by performing a five-point calibration curve (0.016-0.25 mg/mL for each analyte) followed by linear regression analysis. This analysis was performed with Microsoft Excel (Redmond, Wash.) software. The calibration curves were generated by injecting each standard in triplicate and the RSDs provided the measure of precision while the absolute accuracy was determined by comparing the concentrations of the standards predicted by the calibration curve to their “known” values determined by dilution ratios. AOAC International standards for accuracy and precision were used as quality guidelines for every calibration. Check standards were run at the start, middle, and end of every analysis, and recalibration was performed when they varied more than +/−5% of their initial average response.

iv. Orthogonal Analyses of all Samples

The cannabinoid content was quantified by both GC-FID and HPLC. The main difference between GC and HPLC is that GC involves thermal stress and mainly resolves analytes by boiling points while HPLC does not involve heat and mainly resolves analytes by polarity. There are several reasons that this orthogonal approach to analyses is desirable for highly accurate and reproducible results in determining chemotype. The first reason is related to the difference between the cannabinoids produced naturally by the plant (the acidic cannabinoids) and those that are bioactive (the neutral cannabinoids). Cannabis biosynthesizes all the cannabinoids in their relatively unstable acidic forms, and these forms are generally not bioactive in the traditional sense. The application of heat (flame, vaporizer, oven, etc.) causes a loss of the carboxylic acid group and generates the neutral forms of the cannabinoids, which are generally the bioactive forms that are sought after, however this process is highly variable and not quantitative. If one wants to know the native phytochemical profile of the plant then HPLC should be used since this assay does not involve heat. If one wants to know the possible available amount of bioactive cannabinoids, then GC should be used since conversion to these forms in the injector of the GC is an inherent part of the analytical method.

The second reason is also related to the difference between the acidic and neutral cannabinoids, but has to do with the availability of analytical standards to calibrate the instruments. While all of the neutral cannabinoids (THC, CBG, CBC, CBD, and CBN) are available as analytical standards, THCA is the only acidic cannabinoid available as an analytical standard and the instruments were only calibrated for quantification using actual analytical standards. Technically the HPLC assay could characterize the naturally occurring chemotypes, but the acidic analytes are not available as standards, so this quantification is approximate and considered for information only. The acidic analytes are all quantified by reference to the calibration curve for THCA, and this is not an unreasonable assumption as many of them have approximately the same spectral properties. The GC assay is calibrated with analytical standards, but these are the neutral cannabinoids and their formation from the naturally occurring acidic cannabinoids in the GC injector is not quantitative, which complicates exact characterization of the naturally occurring chemotype.

The final reason is simply to have an internal crosscheck of our results by using orthogonal testing methods. Each type of assay (GC and HPLC) has its strengths and weaknesses, and by using both methods, one can compare results and ensure that both the identification and quantitation of the components are accurate. A caveat to this, as mentioned above, is that the conversion of the acidic forms to the neutral forms is not quantitative due to thermal degradation. Under the highly optimized conditions of a GC injector, we have found conversion can vary between 75-85% (for analytical THCA standards), while cannabis samples generally have a conversion of 70-80%. Similar conversion rates are also described in literature for highly optimized analytical instruments (Dussy et al. 2004). Because of this incomplete conversion our GC results are consistently 20-30% lower than the HPLC results for cannabis samples. This same conversion efficiency can be applied to estimate the maximum availability of THC based on THCA content when smoking or vaporizing cannabis.

v. Method “Validation”

Method validation is important in establishing that a method is fit for its intended purpose, providing assurance that the results that are reported are precise, accurate, and reflective of the sample. Very few labs in the cannabis industry attempt to validate their assays and this fact, combined with inappropriate sampling have resulted in erroneous data for several varieties. In order to validate the analytical methods employed for this project, an abbreviated protocol similar to Single Laboratory Validation (SLV) was carried out. Assay “validation” was carried out by spiking blank matrix with the analytes at low, med, and high concentrations and carrying out the assay procedure in replicate (n=5). While some analytes provided better results than others the analyte RSDs, recoveries, and precisions at these concentrations satisfied AOAC guidance (based on mg/mL). In general, the RSDs for the terpenes at the low, medium, and high concentrations (varied by terpene but generally 0.016, 0.125, and 1.0 mg/mL) were less than 5%, 4%, and 3% respectively. The absolute bias for these analytes was generally less than 10%, 4%, and 2%. In general the RSDs for the cannabinoids by both GC and HPLC at the low, medium, and high concentrations (0.016, 0.61, and 0.250 mg/mL) were less than 2%, 2%, and 1% respectively. The absolute bias for these analytes was generally less than 10%, 2%, and 2%. The assays all provided satisfactory S/N ratios at the lowest level and this was initially taken as the LOQ. After subsequent re-calibrations (n=3 at each level), the LOQ was taken as the lowest level of the calibration curve that provided acceptable accuracy (<10% error) determined by comparing the known concentration levels (determined by dilution ratios) to the predicted levels (obtained from the signal and calibration curve).

The error between the known and measured values establishes the accuracy of the method and verifies that real samples do not present any matrix effects that influence the resulting measurements. The precision, or closeness of individual measurements, of the method is also determined by carrying out all analyses in replicate (n=5). Guidance for acceptable values was taken from publications provided by the AOAC.

The in-house validation revealed that the above-described chemical analysis methods were accurate and reliable, and the use of orthogonal methods of analyses provided an internal check on the assays as well as an understanding of the use of GC to analyze thermally unstable molecules. Using multiple dilution ratios kept samples in the linear ranges of the assays, and method validation verified that precise and accurate results were obtained.

Similar methods for analyzing cannabinoids and terpenes are also discussed I Giese et al. “Development and Validation of a Reliable and Robust Method for the Analysis of Cannabinoids and Terpenes in Cannabis” Journal of AOAC International Vol. 98 No. 6, 2015, incorporated herein by reference. See also U.S. patent application Ser. No. 15/539,344, which is hereby incorporated by reference.

### Example 2. Volunteer Trials Using CBG Enhanced Cannabis. (PROPHETIC)

In order to further demonstrate the added utility of the CBG Specialty Cannabis varieties of the present invention, volunteer comparison trials will conducted. During these trials, volunteers will be provided with cannabis blends with varying terpene and cannabinoid profiles to determine the effect of Specialty Cannabis with higher CBG content.

The volunteer trial for CBG will be conducted over 2 weeks. Volunteers will be split into six groups (1-6). Each volunteer in the group will be given two samples (a control and a comparator blend). In this trial, the control comparator blends will be prepared to contain nearly identical levels of a non-CBG cannabinoid (e.g. THC, and/or CBD), but each week the comparator will be formulated so as to include different levels of CBG added (e.g., either 2%, 5%, or 7.5% CBG added in).

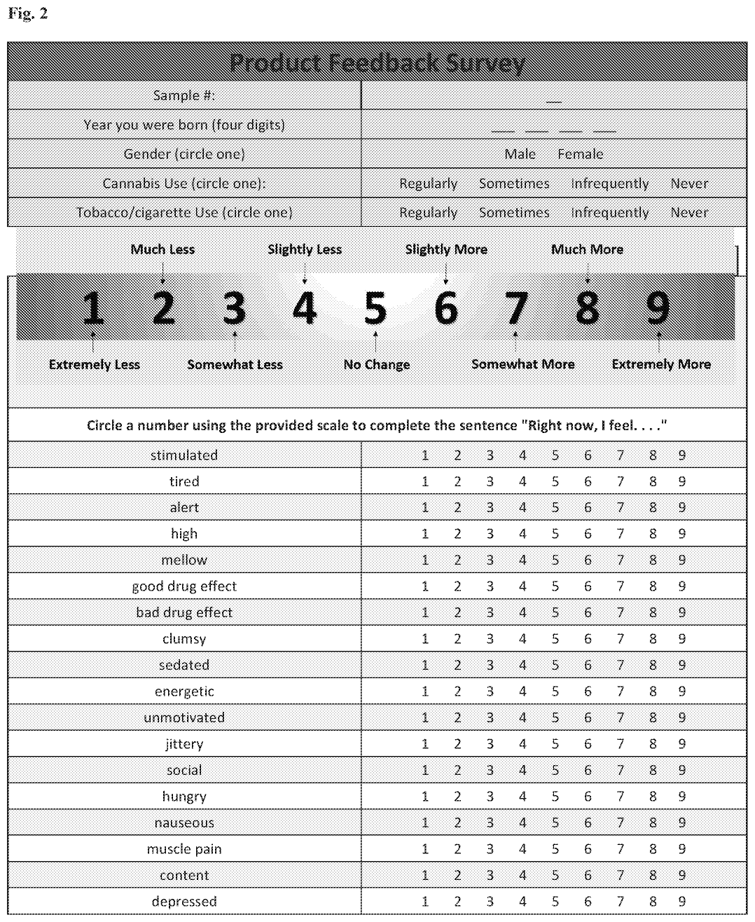

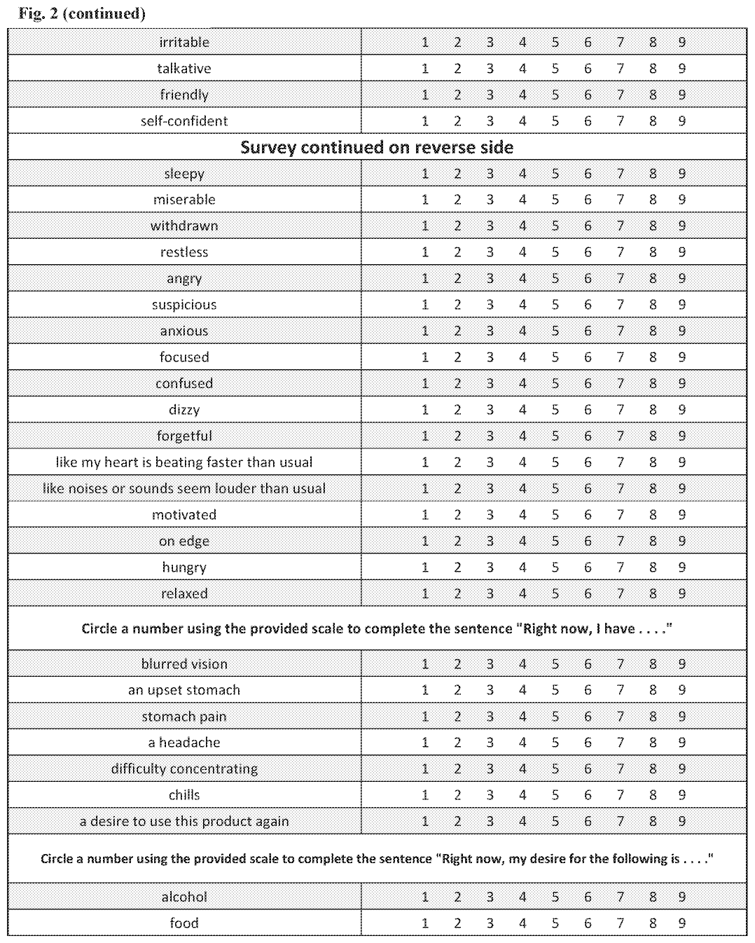

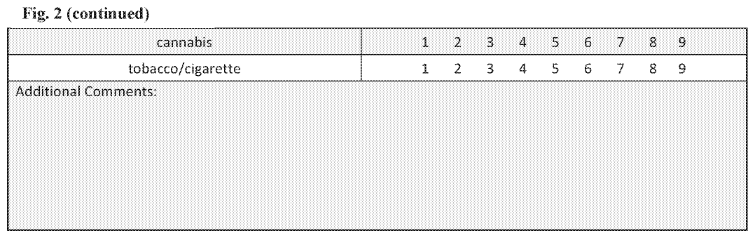

Thirty volunteers will be recruited and asked to fill out surveys inquiring about the experience of smoking/vaporizing each sample. Surveys will also ask volunteers questions related to their physiological response to the sample. An example of the type of questionnaire that will be used is shown in FIG. 2.

### Example 3. Volunteer Trials Using CBG Enhanced Cannabinoid Compositions. (PROPHETIC)

Volunteer comparison trials will be conducted to further determine the effect of increased CBG content in cannabinoid compositions. Volunteer trials will be conducted in similar fashion to those of Example 2.

Briefly, each volunteer in the group will be given two composition samples (a control and a comparator blend). The samples will be provided in single-use e-cigarettes or in tinctures or other formulations designed to be vaporized or administered to the mucosa/swallowed, respectively. In this trial, the control comparator blends will be prepared to contain nearly identical levels of a non-CBG cannabinoid (e.g. THC, and/or CBD), but each week the comparator will be formulated so as to include different levels of CBG added (e.g., either 2%, 5%, or 7.5% CBG added in).

Thirty volunteers will be recruited and asked to fill out surveys inquiring about the experience of smoking each sample. Surveys will also ask volunteers questions related to their physiological response to the sample. An example of the type of questionnaire that will be used is shown in FIG. 2.

### Example 4 Analysis of CBG Parental Varieties

One objective of the inventions of the present disclosure was to develop cannabis varieties accumulating non-trace or high levels of CBG and/or CBGV (“CBG/CBGv”), together with non-trace or high levels of non-CBG cannabinoids. This goal was achieved through a multi-pronged cannabis breeding program that utilized existing public and proprietary cannabis lines to produce novel cannabis germplasms exhibiting high levels of CBG/CBGv across varied genetic and phenotypic backgrounds.

As an initial step, the cannabinoid profiles of each CBG parental variety were determined using HPLC as described in Example 1. The resulting measurements of initial parental lines GRN01, BLK03, ORA03, RED08, SLV09, GLD02, PUR01, and CBD05 are summarized in Table 3.

All of the initial parental lines exhibited less than 1.65% of total CBG/CBGv max contents, with the majority of parental lines exhibiting less than 1.0% total CBG/CBGv max contents. Parental lines that comprised functional B_{D} alleles and accumulated non-trace levels of CBD, exhibited even lower CBG contents. Parental line CBD05 for example, only accumulated 0.26% CBG max.

Table 3 also reports the cannabinoid contents of intermediate filial generations generated during each breeding scheme that were used as parents for the final progeny lines.

TABLE 3
Cannabinoid Contents of CBG Parental Lines

| Parental | | | | | | | | | | |
|---|---|---|---|---|---|---|---|---|---|---|
| Name | THCA | CBDA | CBGA | CBCA | THCVA | CBDVA | CBGVA | THC | CBD | CBG |
| GRN01 | 15.76% | 0.00% | 1.37% | 0.00% | 0.13% | 0.00% | 0.00% | 0.54% | 0.00% | 0.08% |
| BLK03 | 12.18% | 0.10% | 1.23% | 0.00% | 0.00% | 0.00% | 0.00% | 0.35% | 0.00% | 0.00% |
| GN1.B3.24 | 20.84% | 0.00% | 2.41% | 0.00% | 0.17% | 0.00% | 0.00% | 0.37% | 0.00% | 0.12% |
| ORA03 | 9.74% | 0.00% | 0.10% | 0.00% | 0.16% | 0.00% | 0.00% | 0.30% | 0.00% | 0.00% |
| O3.J21.52 | 16.69% | 0.00% | 1.38% | 0.00% | 0.00% | 0.00% | 0.00% | 4.59% | 0.00% | 0.21% |
| SLV09 | 14.91% | 0.00% | 1.07% | 0.00% | 0.00% | 0.00% | 0.00% | 0.00% | 0.00% | 0.00% |
| R8.S9.02 | 9.42% | 0.41% | 0.82% | 0.17% | 0.00% | 0.00% | 0.00% | 0.18% | 0.00% | 0.00% |
| R8.S9.03 | 10.47% | 0.00% | 0.92% | 0.21% | 0.00% | 0.00% | 0.00% | 0.24% | 0.00% | 0.00% |
| GLD02 | 24.53% | 0.00% | 0.88% | 0.00% | 0.29% | 0.00% | 0.00% | 1.70% | 0.00% | 0.11% |
| PUR01 | 14.74% | 0.00% | 0.24% | N/A | N/A | N/A | N/A | 0.31% | 0.00% | 0.00% |
| CBD05 | 5.24% | 10.77% | 0.30% | N/A | N/A | N/A | N/A | 0.20% | 0.14% | 0.00% |
| P1.C5.10 | 0.49% | 13.44% | 1.84% | 0.00% | 0.00% | 0.00% | 0.00% | 0.00% | 0.58% | 0.24% |
| P1.C5.04 | 0.40% | 10.53% | 0.74% | 0.00% | 0.00% | 0.00% | 0.00% | 0.00% | 0.48% | 0.13% |
| PP03 | 0.50% | 13.49% | 1.11% | 0.71% | 0.00% | 0.00% | 0.00% | 0.00% | 0.19% | 0.18% |
| P08 | 6.79% | 6.23% | 0.26% | 0.00% | 0.00% | 0.00% | 0.00% | 0.07% | 0.00% | 0.00% |
| RED08 | 10.77% | 0.00% | 1.78% | 0.00% | 0.00% | 0.00% | 0.00% | 0.38% | 0.00% | 0.08% |

| | | | | | | | | Total max |
|---|---|---|---|---|---|---|---|---|
| Parental | | | | | | | TOTAL | Non-CBG |
| Name | CBC | THCV | CBDV | CBGV | CBN | D8-THC | CBG/CBGVmax* | Cannabinoids* |
| GRN01 | 0.00% | 0.00% | 0.00% | 0.00% | 0.00% | 0.00% | 1.28% | 14.48% |
| BLK03 | 0.00% | 0.00% | 0.00% | 0.00% | 0.00% | 0.00% | 1.08% | 11.12% |
| GN1.B3.24 | 0.00% | 0.00% | 0.00% | 0.00% | 0.00% | 0.00% | 2.24% | 18.81% |
| ORA03 | 0.00% | 0.00% | 0.00% | 0.00% | 0.00% | 0.00% | 0.08% | 8.98% |
| O3.J21.52 | 0.00% | 0.00% | 0.00% | 0.00% | 0.00% | 0.00% | 1.42% | 19.22% |
| SLV09 | 0.00% | 0.00% | 0.00% | 0.00% | 0.00% | 0.00% | 0.94% | 13.08% |
| R8.S9.02 | 0.00% | 0.00% | 0.00% | 0.00% | 0.00% | 0.00% | 0.72% | 8.95% |
| R8.S9.03 | 0.00% | 0.00% | 0.00% | 0.00% | 0.00% | 0.00% | 0.81% | 9.60% |
| GLD02 | 0.00% | 0.00% | 0.00% | 0.00% | 0.00% | 0.00% | 0.89% | 23.47% |
| PUR01 | N/A | N/A | N/A | N/A | N/A | N/A | 0.21% | 13.24% |
| CBD05 | N/A | N/A | N/A | N/A | N/A | N/A | 0.26% | 14.38% |
| P1.C5.10 | 0.00% | 0.00% | 0.00% | 0.00% | 0.00% | 0.00% | 1.86% | 12.80% |
| P1.C5.04 | 0.00% | 0.00% | 0.00% | 0.00% | 0.00% | 0.00% | 0.78% | 10.06% |
| PP03 | 0.00% | 0.00% | 0.00% | 0.00% | 0.00% | 0.00% | 1.16% | 13.08% |
| P08 | 0.00% | 0.00% | 0.00% | 0.00% | 0.00% | 0.00% | 0.22% | 11.49% |
| RED08 | 0.00% | 0.00% | 0.00% | 0.00% | 0.00% | 0.00% | 1.64% | 9.83% |

*All Max values were calculated based on theoretical maximum formulas disclosed in this application. N/A indicates that the cannabinoid was not tested.

### Example 5 Breeding of Specialty Cannabis

One of the objectives of the breeding programs of the present disclosure was to produce plants that expressed non-trace levels of CBG/CBGV with high levels of non-CBG cannabinoids NGCs. In some embodiments, the Specialty Cannabis varieties of the present invention were additionally selected for their ability to produce terpenes that are appealing to patients and that may also provide a pharmacological activity that modifies, enhances or ameliorates the effects of the cannabinoids. Thus, a secondary objective of the breeding programs of the present disclosure was to produce plants with high terpene oil content and diverse Terpene Profiles.

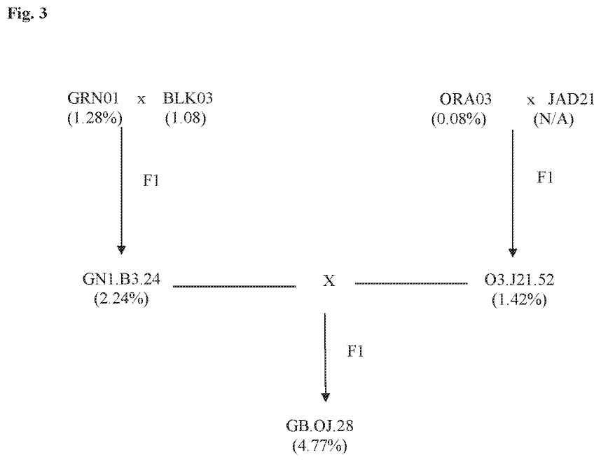

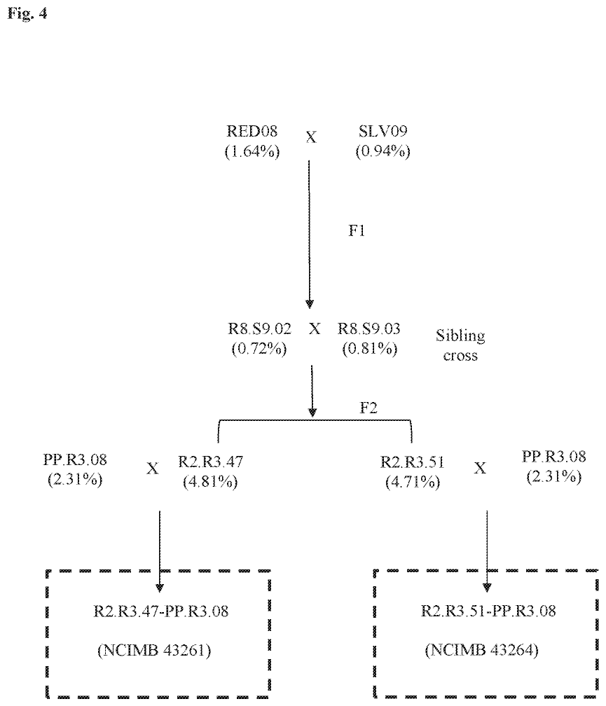

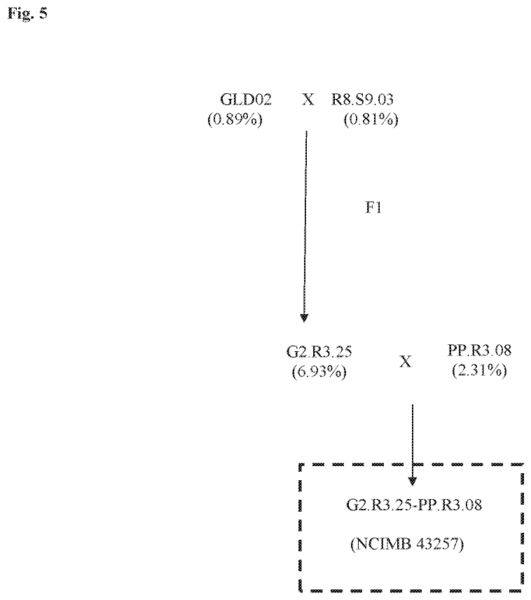

In order to achieve these objectives the parental cannabis varieties of Example 4 were incorporated into a multi-pronged cannabis breeding program to develop Specialty Cannabis plants and varieties. FIGS. 3-7 depict the breeding schemes for five exemplary independently produced high CBG varieties of the present disclosure.

The breeding schemes of FIGS. 3-5 were designed to increase CBG production in B_{T}/B_{T} cannabis lines. These schemes resulted in novel cannabis varieties exhibiting three to eight-fold increases in CBG max contents. In some instances, the resulting progeny also exhibited high oil contents and varied terpene phenotypes.

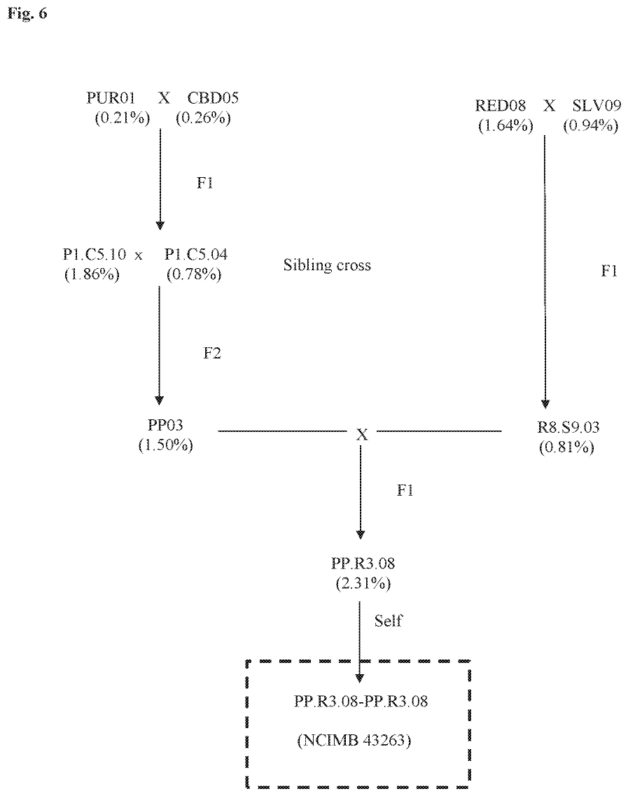

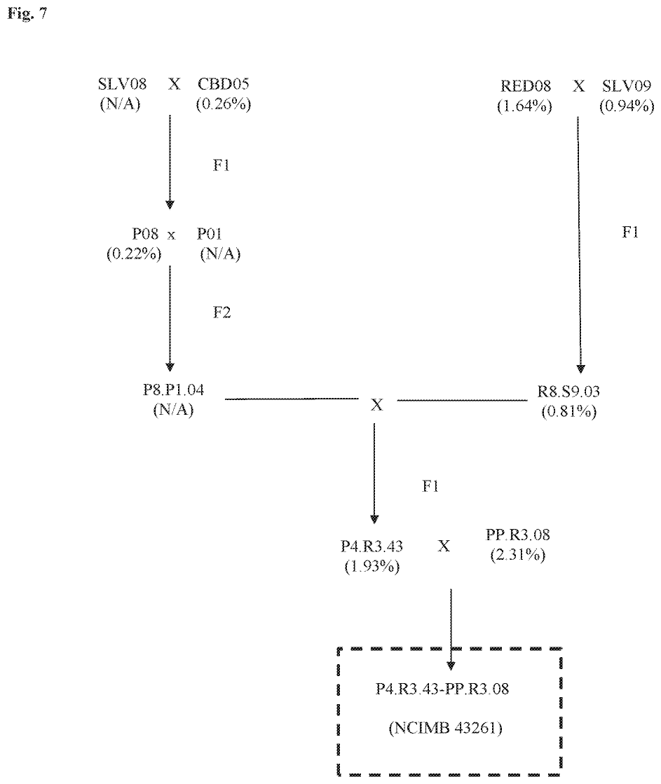

The breeding schemes of FIGS. 6-7 were designed to increase CBG production in B_{T}/B_{D} or B_{D}/B_{D} genotypes. Traditionally, plants comprising even a single B_{D} allele had exhibited little to no CBG or CBGV. Without wishing to be bound by any single theory, the present inventors believe that the lack of CBG/CBGv accumulation in plants with B_{D} allele(s) is due to the high productivity of CBDA synthase enzymes, which deplete the source of CBG/CBGv precursors.

The present disclosure envisions further crosses from those described in FIGS. 3-7. In one representative version of this breeding regime the resultant F1 progeny from the crosses of any one of FIGS. 3-7, or Tables 5-16 can be selfed to bulk up F2 seed. In some embodiments, the F2 seed will be used for biological deposit.

F2 seed can further be grown to produce F2 progeny. Selection for desirable phenotypes and/or genotypes can be conducted within the F1, F2, or subsequent progeny since the selections can be maintained (i.e., fixed) via asexual reproduction. Alternatively, the F2 progeny can be crossed among themselves to produce a bulked F3 population from which desired progeny can be selected and/or further generations of crossing can be conducted. Again, selected F2 progeny can be maintained (i.e., fixed) via asexual reproduction. In another embodiment, the resultant F1 progeny can by backcrossed to high CBG parent or the NGC variety to further reinforce the traits of other parent. In yet another representative version of this breeding scheme F1, F2, or subsequent progeny may also be crossed to additional NGCs varieties to create even more complex cannabinoid combinations.

For example, regardless of the exact crossing/selection procedure, selected lines can be chosen so as to have a total CBG/CBGV content of >2.5%, or >3.0%, or >5.0%, and a total NGCs content >3.0%, >6.0%, >9.0%. In some embodiments, the selected lines are bred to produce desirable aroma and flavor profiles.

According to the present invention, the lines can also be further selected for a specific content of certain other cannabinoids and/or of certain terpenes/terpenoids, and/or for additional phenotypic and genotypic characteristics. Desirable phenotypic characteristics include but are not limited to larger plant size (i.e., greater bulk or biomass), higher production of flower buds, larger flowers, more trichomes, shorter plant stature, ability to tolerate lower and/or higher growing temperatures, greater germination percentage, greater seedling vigor, more efficient water usage, disease resistance, pest resistance, and other desirable agronomic and production traits. For an overview of diseases and pests of importance to cannabis production see Clarke et al. (2000) Hemp Diseases and Pests: Management and Biological Control: An Advanced Treatise (CABI Publishing).

The progeny resulting from any selection stage of either the crosses described in FIGS. 3-7, or any of the described selfing, sib crosses, or backcrossing versions of the breeding regimes of the present invention can be asexually reproduced so as to fix and maintain the desirable content, CBG content, NGCs content, the aroma and flavor(s) typical of the desired class, and the other desirable phenotypic and/or genotypic characteristics. The resultant selected lines will be designated as Specialty Cannabis Varieties.

The resultant Specialty Cannabis plants of the present invention also generally have more terpene essential oil content per plant than contemporary marijuana varieties. More essential oil per plant means less plant matter is required per treatment/administration, thereby also further minimizing any health risks for medical and recreational cannabis smokers. This would also further increase production efficiency.

### Example 6. Chemical Analysis of Specialty Cannabis

The new Specialty Cannabis varieties created through crosses as described in Example 5 were subjected to cannabinoid and terpene chemical analysis as described in Example 1. The resulting breeding schemes of Example 5produced five separate lines of high CBG cannabis germplasm (GB.OJ, R2.R3, G2.R3, PP.R3, and P4.R3). The level of cannabinoids for each of the high CBG lines was measured by HPLC, and is presented across Tables 5-9. Terpenes were measured using GC-FID, and are presented as absolute content measurements based on the percent content by weight of dry inflorescences in Tables 10-14. A summary table of representative lines from each of the high CBG lines is presented in Table 4.

TABLE 4
Representative high CBG lines of the present disclosure

| | | Non-CBG | | |
|---|---|---|---|---|
| | | Cannabinoid | | |
| | CBG/CBGv | Max (NGCs | | Dominant |
| High CBG Line | max | max) | Chemotype | Terpenes |
| GB.OJ | 4.77% | 15.99% | Type I | Beta |
| | | | | Caryophyllene |
| R2.R3 | 8.27% | 12.75% | Type I | Beta Ocimene |
| Half-sib bulked | | | | |
| seed deposited | | | | |
| as: | | | | |
| (NCIMB 43261) | | | | |
| and | | | | |
| (NCIMB 43264) | | | | |
| G2.R3 | 6.93% | 15.88% | Type I | Beta Ocimene |
| Half-sib bulked | | | | |
| seed deposited | | | | |
| as: | | | | |
| (NCIMB 43257) | | | | |
| PP.R3 | 2.31% | 18.47% | Type II | Beta |
| Selfed bulked | | | | Caryophyllene |
| seed deposited | | | | |
| as | | | | |
| (NCIMB 43263) | | | | |
| P4.R3 | 2.03% | 20.8% | Type II | Myrcene |
| Half-sib bulked | | | | |
| seed deposited | | | | |
| as: | | | | |
| (NCIMB 43261) | | | | |

TABLE 5
Cannabinoid Contents of GB.OJ high CBG line

| Variety | | | | | | | | | | |
|---|---|---|---|---|---|---|---|---|---|---|
| Name | THCA | CBDA | CBGA | CBCA | THCVA | CBDVA | CBGVA | THC | CBD | CBG |
| GB.OJ.28-1 | 10.25% | 0.00% | 1.83% | 0.37% | 0.00% | 0.00% | 0.00% | 0.00% | 0.00% | 0.07% |
| GB.OJ.28-2 | 16.92% | 0.00% | 5.27% | 0.87% | 0.19% | 0.00% | 0.00% | 0.23% | 0.00% | 0.14% |
| GB.OJ.28-3 | 17.01% | 0.00% | 2.69% | 0.18% | 0.14% | 0.00% | 0.00% | 0.42% | 0.00% | 0.16% |
| GB.OJ.28-4 | 10.19% | 0.00% | 2.85% | 0.07% | 0.00% | 0.00% | 0.00% | 0.17% | 0.00% | 0.07% |
| GB.OJ.28-5 | 13.79% | 0.00% | 3.56% | 0.19% | 0.00% | 0.00% | 0.00% | 0.26% | 0.00% | 0.09% |
| GB.OJ.28-6 | 16.69% | 0.00% | 4.22% | 0.28% | 0.17% | 0.00% | 0.00% | 0.25% | 0.00% | 0.11% |
| GB.OJ.28-7 | 19.53% | 0.00% | 3.57% | 0.64% | 0.15% | 0.00% | 0.00% | 0.18% | 0.00% | 0.11% |
| GB.OJ.28-8 | 17.08% | 0.00% | 5.09% | 0.00% | 0.00% | 0.00% | 0.00% | 0.14% | 0.00% | 0.09% |
| GB.OJ.28-9 | 15.44% | 0.00% | 5.00% | 0.00% | 0.15% | 0.00% | 0.00% | 0.60% | 0.00% | 0.15% |

| | | | | | | | | Total max |
|---|---|---|---|---|---|---|---|---|
| Variety | | | | | | | TOTAL | Non-CBG |
| Name | CBC | THCV | CBDV | CBGV | CBN | D8-THC | CBG/CBGVmax* | Cannabs* |
| GB.OJ.28-1 | 0.00% | 0.00% | 0.00% | 0.00% | 0.00% | 0.00% | 1.68% | 9.32% |
| GB.OJ.28-2 | 0.00% | 0.00% | 0.00% | 0.00% | 0.00% | 0.00% | 4.77% | 15.99% |
| GB.OJ.28-3 | 0.00% | 0.00% | 0.00% | 0.00% | 0.00% | 0.00% | 2.52% | 15.62% |
| GB.OJ.28-4 | 0.00% | 0.00% | 0.00% | 0.00% | 0.00% | 0.00% | 2.58% | 9.17% |
| GB.OJ.28-5 | 0.00% | 0.00% | 0.00% | 0.00% | 0.00% | 0.00% | 3.21% | 12.52% |
| GB.OJ.28-6 | 0.00% | 0.00% | 0.00% | 0.00% | 0.00% | 0.00% | 3.82% | 15.29% |
| GB.OJ.28-7 | 0.00% | 0.00% | 0.00% | 0.00% | 0.00% | 0.00% | 3.24% | 18.00% |
| GB.OJ.28-8 | 0.00% | 0.00% | 0.00% | 0.00% | 0.00% | 0.00% | 4.57% | 15.12% |
| GB.OJ.28-9 | 0.00% | 0.00% | 0.00% | 0.00% | 0.00% | 0.00% | 4.54% | 14.28% |

*ALL Max values were calculated based on theoretical maximum formulas disclosed in this application.

TABLE 6
Cannabinoid Contents of R2.R3 high CBG line

| Variety | | | | | | | | | | |
|---|---|---|---|---|---|---|---|---|---|---|
| Name | THCA | CBDA | CBGA | CBCA | THCVA | CBDVA | CBGVA | THC | CBD | CBG |
| R2.R3.24 | 13.99% | 0.00% | 9.16% | 0.00% | 0.15% | 0.00% | 0.00% | 0.35% | 0.00% | 0.23% |
| R2.R3.47 | 15.38% | 0.00% | 5.32% | 0.00% | 0.14% | 0.00% | 0.00% | 0.29% | 0.00% | 0.14% |
| R2.R3.08 | 17.80% | 0.00% | 5.30% | 0.00% | 0.15% | 0.00% | 0.00% | 0.29% | 0.00% | 0.16% |
| R2.R3.51 | 18.70% | 0.00% | 5.18% | 0.00% | 0.14% | 0.00% | 0.00% | 0.50% | 0.00% | 0.15% |
| R2.R3.05 | 15.22% | 0.00% | 4.98% | 0.00% | 0.00% | 0.00% | 0.00% | 0.24% | 0.00% | 0.16% |
| R2.R3.15 | 15.22% | 0.00% | 4.94% | 0.00% | 0.20% | 0.00% | 0.00% | 0.30% | 0.00% | 0.11% |
| R2.R3.70 | 11.73% | 0.00% | 4.77% | 0.00% | 0.00% | 0.00% | 0.00% | 0.42% | 0.00% | 0.10% |
| R2.R3.16 | 12.89% | 0.00% | 4.70% | 0.00% | 0.00% | 0.00% | 0.00% | 0.32% | 0.00% | 0.15% |
| R2.R3.61 | 13.04% | 0.00% | 4.08% | 0.00% | 0.16% | 0.00% | 0.00% | 0.58% | 0.00% | 0.10% |
| R2.R3.55 | 18.25% | 0.00% | 4.06% | 0.00% | 0.22% | 0.00% | 0.00% | 0.33% | 0.00% | 0.12% |
| R2.R3.49 | 21.41% | 0.08% | 3.94% | 0.00% | 0.23% | 0.00% | 0.00% | 0.38% | 0.00% | 0.14% |
| R2.R3.06 | 20.19% | 0.00% | 3.94% | 0.00% | 0.19% | 0.00% | 0.00% | 0.27% | 0.00% | 0.13% |
| R2.R3.66 | 14.18% | 0.00% | 3.82% | 0.00% | 0.16% | 0.00% | 0.00% | 0.60% | 0.00% | 0.15% |
| R2.R3.64 | 15.80% | 0.00% | 3.81% | 0.00% | 0.00% | 0.00% | 0.00% | 0.39% | 0.00% | 0.10% |
| R2.R3.42 | 16.45% | 0.00% | 3.79% | 0.00% | 0.00% | 0.00% | 0.00% | 0.46% | 0.00% | 0.11% |
| R2.R3.56 | 16.30% | 0.00% | 3.78% | 0.00% | 0.17% | 0.00% | 0.00% | 0.33% | 0.00% | 0.13% |
| R2.R3.60 | 18.08% | 0.00% | 3.72% | 0.00% | 0.17% | 0.00% | 0.00% | 0.37% | 0.00% | 0.15% |
| R2.R3.62 | 15.07% | 0.00% | 3.64% | 0.00% | 0.14% | 0.00% | 0.00% | 0.40% | 0.00% | 0.10% |
| R2.R3.39 | 17.23% | 0.00% | 3.50% | 0.00% | 0.20% | 0.00% | 0.00% | 0.28% | 0.00% | 0.12% |
| R2.R3.41 | 12.48% | 0.00% | 3.35% | 0.00% | 0.18% | 0.00% | 0.00% | 0.27% | 0.00% | 0.09% |
| R2.R3.02 | 16.39% | 0.00% | 3.21% | 0.00% | 0.17% | 0.00% | 0.00% | 1.37% | 0.00% | 0.16% |
| R2.R3.40 | 15.23% | 0.00% | 3.06% | 0.00% | 0.18% | 0.00% | 0.00% | 0.40% | 0.00% | 0.15% |
| R2.R3.21 | 17.19% | 0.00% | 3.02% | 0.00% | 0.00% | 0.00% | 0.00% | 0.36% | 0.00% | 0.12% |
| R2.R3.01 | 15.64% | 0.00% | 3.02% | 0.00% | 0.18% | 0.00% | 0.00% | 0.26% | 0.00% | 0.09% |
| R2.R3.71 | 16.79% | 0.00% | 2.99% | 0.00% | 0.17% | 0.00% | 0.00% | 0.49% | 0.00% | 0.13% |
| R2.R3.45 | 16.95% | 0.00% | 2.97% | 0.00% | 0.19% | 0.00% | 0.00% | 0.25% | 0.00% | 0.13% |
| R2.R3.30 | 14.76% | 0.00% | 2.73% | 0.00% | 0.00% | 0.00% | 0.00% | 0.35% | 0.00% | 0.15% |
| R2.R3.54 | 12.10% | 0.00% | 2.67% | 0.00% | 0.00% | 0.00% | 0.00% | 0.26% | 0.00% | 0.00% |
| R2.R3.28 | 19.11% | 0.00% | 2.60% | 0.00% | 0.21% | 0.00% | 0.00% | 0.46% | 0.00% | 0.09% |

| | | | | | | | | Total max |
|---|---|---|---|---|---|---|---|---|
| Variety | | | | | | | TOTAL | Non-CBG |
| Name | CBC | THCV | CBDV | CBGV | CBN | D8-THC | CBG/CBGVmax* | Cannabs* |
| R2.R3.24 | 0.00% | 0.00% | 0.00% | 0.00% | 0.00% | 0.00% | 8.27% | 12.75% |
| R2.R3.47 | 0.00% | 0.00% | 0.00% | 0.00% | 0.00% | 0.00% | 4.81% | 13.90% |
| R2.R3.08 | 0.00% | 0.00% | 0.00% | 0.00% | 0.00% | 0.00% | 4.81% | 16.03% |
| R2.R3.51 | 0.00% | 0.00% | 0.00% | 0.00% | 0.00% | 0.00% | 4.70% | 17.03% |
| R2.R3.05 | 0.00% | 0.00% | 0.00% | 0.00% | 0.00% | 0.00% | 4.53% | 13.58% |
| R2.R3.15 | 0.00% | 0.00% | 0.00% | 0.00% | 0.00% | 0.00% | 4.45% | 13.82% |
| R2.R3.70 | 0.00% | 0.00% | 0.00% | 0.00% | 0.00% | 0.00% | 4.29% | 10.71% |
| R2.R3.16 | 0.00% | 0.00% | 0.00% | 0.00% | 0.00% | 0.00% | 4.28% | 11.62% |
| R2.R3.61 | 0.00% | 0.00% | 0.00% | 0.00% | 0.00% | 0.00% | 3.68% | 12.16% |
| R2.R3.55 | 0.00% | 0.00% | 0.00% | 0.00% | 0.00% | 0.00% | 3.68% | 16.52% |
| R2.R3.49 | 0.00% | 0.00% | 0.00% | 0.00% | 0.00% | 0.00% | 3.60% | 19.43% |
| R2.R3.06 | 0.00% | 0.00% | 0.00% | 0.00% | 0.00% | 0.00% | 3.58% | 18.14% |
| R2.R3.66 | 0.00% | 0.00% | 0.00% | 0.00% | 0.00% | 0.00% | 3.50% | 13.17% |
| R2.R3.64 | 0.00% | 0.00% | 0.00% | 0.00% | 0.00% | 0.00% | 3.45% | 14.24% |
| R2.R3.42 | 0.00% | 0.00% | 0.00% | 0.00% | 0.00% | 0.00% | 3.44% | 14.89% |
| R2.R3.56 | 0.00% | 0.00% | 0.00% | 0.00% | 0.00% | 0.00% | 3.45% | 14.77% |
| R2.R3.60 | 0.00% | 0.00% | 0.00% | 0.00% | 0.00% | 0.00% | 3.42% | 16.38% |
| R2.R3.62 | 0.00% | 0.00% | 0.00% | 0.00% | 0.00% | 0.00% | 3.29% | 13.74% |
| R2.R3.39 | 0.00% | 0.00% | 0.00% | 0.00% | 0.00% | 0.00% | 3.19% | 15.57% |
| R2.R3.41 | 0.00% | 0.00% | 0.00% | 0.00% | 0.00% | 0.00% | 3.03% | 11.38% |
| R2.R3.02 | 0.00% | 0.00% | 0.00% | 0.00% | 0.00% | 0.00% | 2.98% | 15.89% |
| R2.R3.40 | 0.00% | 0.00% | 0.00% | 0.00% | 0.00% | 0.00% | 2.84% | 13.92% |
| R2.R3.21 | 0.00% | 0.00% | 0.00% | 0.00% | 0.00% | 0.00% | 2.77% | 15.44% |
| R2.R3.01 | 0.00% | 0.00% | 0.00% | 0.00% | 0.00% | 0.00% | 2.74% | 14.14% |
| R2.R3.71 | 0.00% | 0.00% | 0.00% | 0.00% | 0.00% | 0.00% | 2.76% | 15.36% |
| R2.R3.45 | 0.00% | 0.00% | 0.00% | 0.00% | 0.00% | 0.00% | 2.74% | 15.29% |
| R2.R3.30 | 0.00% | 0.00% | 0.00% | 0.00% | 0.00% | 0.00% | 2.54% | 13.29% |
| R2.R3.54 | 0.00% | 0.00% | 0.00% | 0.00% | 0.00% | 0.00% | 2.35% | 10.87% |
| R2.R3.28 | 0.00% | 0.00% | 0.00% | 0.00% | 0.00% | 0.00% | 2.38% | 17.41% |

*All Max values were calculated based on theoretical maximum formulas disclosed in this application.

TABLE 7
Cannabinoid Contents of G2.R3 high CBG line

| Variety | | | | | | | | | | |
|---|---|---|---|---|---|---|---|---|---|---|
| Name | THCA | CBDA | CBGA | CBCA | THCVA | CBDVA | CBGVA | THC | CBD | CBG |
| G2.R3.25 | 17.13% | 0.00% | 7.89% | 0.00% | 0.18% | 0.00% | 0.00% | 0.71% | 0.00% | 0.00% |
| G2.R3.04 | 20.04% | 0.00% | 6.03% | 0.00% | 0.21% | 0.00% | 0.00% | 0.60% | 0.00% | 0.09% |
| G2.R3.15 | 16.67% | 0.00% | 4.45% | 0.00% | 0.16% | 0.00% | 0.00% | 0.37% | 0.00% | 0.10% |
| G2.R3.11 | 17.27% | 0.00% | 4.39% | 0.00% | 0.19% | 0.00% | 0.00% | 0.29% | 0.00% | 0.00% |
| G2.R3.09 | 18.15% | 0.00% | 3.76% | 0.00% | 0.18% | 0.00% | 0.00% | 0.48% | 0.00% | 0.09% |
| G2.R3.07 | 15.18% | 0.00% | 3.54% | 0.00% | 0.00% | 0.00% | 0.00% | 0.44% | 0.00% | 0.10% |
| G2.R3.18 | 17.65% | 0.00% | 3.12% | 0.00% | 0.00% | 0.00% | 0.00% | 0.45% | 0.00% | 0.10% |
| G2.R3.25 | 10.58% | 0.00% | 2.75% | 0.00% | 0.00% | 0.00% | 0.00% | 0.00% | 0.00% | 0.00% |
| G2.R3.13 | 13.65% | 0.00% | 2.50% | 0.00% | 0.00% | 0.00% | 0.00% | 0.67% | 0.00% | 0.10% |

| | | | | | | | | Total max |
|---|---|---|---|---|---|---|---|---|
| Variety | | | | | | | TOTAL | Non-CBG |
| Name | CBC | THCV | CBDV | CBGV | CBN | D8-THC | CBG/CBGVmax* | Cannabs* |
| G2.R3.25 | 0.00% | 0.00% | 0.00% | 0.00% | 0.00% | 0.00% | 6.93% | 15.88% |
| G2.R3.04 | 0.00% | 0.00% | 0.00% | 0.00% | 0.00% | 0.00% | 5.39% | 18.36% |
| G2.R3.15 | 0.00% | 0.00% | 0.00% | 0.00% | 0.00% | 0.00% | 4.01% | 15.13% |
| G2.R3.11 | 0.00% | 0.00% | 0.00% | 0.00% | 0.00% | 0.00% | 3.85% | 15.60% |
| G2.R3.09 | 0.00% | 0.00% | 0.00% | 0.00% | 0.00% | 0.00% | 3.40% | 16.55% |
| G2.R3.07 | 0.00% | 0.00% | 0.00% | 0.00% | 0.00% | 0.00% | 3.20% | 13.76% |
| G2.R3.18 | 0.00% | 0.00% | 0.00% | 0.00% | 0.00% | 0.00% | 2.84% | 15.93% |
| G2.R3.25 | 0.00% | 0.00% | 0.00% | 0.00% | 0.00% | 0.00% | 2.42% | 9.28% |
| G2.R3.13 | 0.00% | 0.00% | 0.00% | 0.00% | 0.00% | 0.00% | 2.30% | 12.65% |

*All Max values were calculated based on theoretical maximum formulas disclosed in this application.

TABLE 8
Cannabinoid Contents of PP.R3 high CBG line

| Variety | | | | | | | | | | |
|---|---|---|---|---|---|---|---|---|---|---|
| Name | THCA | CBDA | CBGA | CBCA | THCVA | CBDVA | CBGVA | THC | CBD | CBG |
| PP.R3.08 | 7.14% | 13.60% | 2.46% | 0.00% | 0.00% | 0.00% | 0.00% | 0.18% | 0.10% | 0.15% |
| PP.R3.26 | 4.88% | 13.72% | 2.02% | 0.00% | 0.00% | 0.00% | 0.00% | 0.27% | 0.17% | 0.08% |
| PP.R3.46 | 7.42% | 17.28% | 1.86% | 0.00% | 0.00% | 0.10% | 0.00% | 0.21% | 0.15% | 0.13% |
| PP.R3.11 | 7.44% | 14.08% | 1.68% | 0.00% | 0.00% | 0.07% | 0.00% | 0.23% | 0.13% | 0.10% |
| PP.R3.33 | 5.80% | 10.77% | 1.47% | 0.00% | 0.00% | 0.00% | 0.00% | 0.17% | 0.08% | 0.10% |
| PP.R3.41 | 8.75% | 14.61% | 1.46% | 0.00% | 0.00% | 0.00% | 0.00% | 0.28% | 0.14% | 0.14% |
| PP.R3.06 | 5.88% | 13.65% | 1.46% | 0.00% | 0.00% | 0.00% | 0.00% | 0.25% | 0.13% | 0.09% |
| PP.R3.25 | 7.05% | 10.86% | 1.42% | 0.00% | 0.00% | 0.00% | 0.00% | 0.29% | 0.10% | 0.10% |
| PP.R3.40 | 7.74% | 15.19% | 1.32% | 0.00% | 0.00% | 0.00% | 0.00% | 0.33% | 0.16% | 0.17% |
| PP.R3.24 | 8.26% | 15.25% | 1.31% | 0.00% | 0.00% | 0.08% | 0.00% | 0.27% | 0.13% | 0.15% |
| PP.R3.03 | 7.23% | 14.88% | 1.29% | 0.00% | 0.00% | 0.00% | 0.00% | 0.25% | 0.12% | 0.12% |
| PP.R3.37 | 8.38% | 13.65% | 1.28% | 0.00% | 0.00% | 0.00% | 0.00% | 0.20% | 0.08% | 0.16% |
| PP.R3.13 | 8.29% | 14.25% | 1.21% | 0.00% | 0.00% | 0.00% | 0.00% | 0.26% | 0.11% | 0.18% |
| PP.R3.07 | 6.34% | 15.28% | 1.19% | 0.00% | 0.00% | 0.00% | 0.00% | 0.27% | 0.16% | 0.09% |
| PP.R3.31 | 7.29% | 15.99% | 1.17% | 0.00% | 0.00% | 0.00% | 0.00% | 0.21% | 0.10% | 0.23% |
| PP.R3.05 | 5.56% | 11.08% | 1.15% | 0.00% | 0.00% | 0.00% | 0.00% | 0.26% | 0.11% | 0.0850 |
| PP.R3.12 | 7.28% | 11.97% | 1.11% | 0.00% | 0.00% | 0.00% | 0.00% | 0.23% | 0.12% | 0.12% |
| PP.R3.04 | 6.50% | 16.09% | 1.11% | 0.00% | 0.00% | 0.07% | 0.00% | 0.24% | 0.13% | 0.12% |
| PP.R3.39 | 7.75% | 14.54% | 1.11% | 0.00% | 0.00% | 0.00% | 0.00% | 0.24% | 0.12% | 0.12% |
| PP.R3.30 | 8.23% | 12.94% | 1.07% | 0.00% | 0.00% | 0.00% | 0.00% | 0.29% | 0.11% | 0.15% |

| | | | | | | | | Total max |
|---|---|---|---|---|---|---|---|---|
| Variety | | | | | | | TOTAL | Non-CBG |
| Name | CBC | THCV | CBDV | CBGV | CBN | D8-THC | CBG/CBGVmax* | Cannabs* |
| PP.R3.08 | 0.00% | 0.00% | 0.00% | 0.00% | 0.00% | 0.00% | 2.31% | 18.47% |
| PP.R3.26 | 0.00% | 0.00% | 0.00% | 0.00% | 0.00% | 0.00% | 1.85% | 16.75% |
| PP.R3.46 | 0.00% | 0.00% | 0.00% | 0.00% | 0.00% | 0.00% | 1.76% | 22.10% |
| PP.R3.11 | 0.00% | 0.00% | 0.00% | 0.00% | 0.00% | 0.00% | 1.57% | 19.30% |
| PP.R3.33 | 0.00% | 0.00% | 0.00% | 0.00% | 0.00% | 0.00% | 1.39% | 14.78% |
| PP.R3.41 | 0.00% | 0.00% | 0.00% | 0.00% | 0.00% | 0.00% | 1.42% | 20.91% |
| PP.R3.06 | 0.00% | 0.00% | 0.00% | 0.00% | 0.00% | 0.00% | 1.37% | 17.50% |
| PP.R3.25 | 0.00% | 0.00% | 0.00% | 0.00% | 0.00% | 0.00% | 1.34% | 16.09% |
| PP.R3.40 | 0.00% | 0.00% | 0.00% | 0.00% | 0.00% | 0.00% | 1.33% | 20.60% |
| PP.R3.24 | 0.00% | 0.00% | 0.00% | 0.00% | 0.00% | 0.00% | 1.30% | 21.09% |
| PP.R3.03 | 0.00% | 0.00% | 0.00% | 0.00% | 0.00% | 0.00% | 1.26% | 19.75% |
| PP.R3.37 | 0.00% | 0.00% | 0.00% | 0.00% | 0.00% | 0.00% | 1.29% | 19.60% |
| PP.R3.13 | 0.00% | 0.00% | 0.00% | 0.00% | 0.00% | 0.00% | 1.24% | 20.14% |
| PP.R3.07 | 0.00% | 0.00% | 0.00% | 0.00% | 0.00% | 0.00% | 1.13% | 19.40% |
| PP.R3.31 | 0.00% | 0.00% | 0.00% | 0.00% | 0.00% | 0.00% | 1.26% | 20.72% |
| PP.R3.05 | 0.00% | 0.00% | 0.00% | 0.00% | 0.00% | 0.00% | 1.09% | 14.96% |
| PP.R3.12 | 0.00% | 0.00% | 0.00% | 0.00% | 0.00% | 0.00% | 1.09% | 17.24% |
| PP.R3.04 | 0.00% | 0.00% | 0.00% | 0.00% | 0.00% | 0.00% | 1.09% | 20.24% |
| PP.R3.39 | 0.00% | 0.00% | 0.00% | 0.00% | 0.00% | 0.00% | 1.09% | 19.91% |
| PP.R3.30 | 0.00% | 0.00% | 0.00% | 0.00% | 0.00% | 0.00% | 1.09% | 18.96% |

*All Max values were calculated based on theoretical maximum formulas disclosed in this application.

TABLE 9
Cannabinoid Contents of P4.R3 high CBG line

| Variety | | | | | | | | | | |
|---|---|---|---|---|---|---|---|---|---|---|
| Name | THCA | CBDA | CBGA | CBCA | THCVA | CBDVA | CBGVA | THC | CBD | CBG |
| P4.R3.74 | 5.79% | 17.22% | 2.04% | 0.00% | 0.00% | 0.08% | 0.00% | 0.31% | 0.24% | 0.24% |
| P4.R3.43 | 8.53% | 15.13% | 2.04% | 0.00% | 0.00% | 0.07% | 0.00% | 0.16% | 0.11% | 0.14% |
| P4.R3.02 | 7.47% | 15.63% | 1.36% | 0.00% | 0.00% | 0.07% | 0.00% | 0.16% | 0.11% | 0.16% |
| P4.R3.23 | 9.93% | 13.78% | 1.09% | 0.00% | 0.00% | 0.07% | 0.00% | 0.21% | 0.10% | 0.24% |
| P4.R3.34 | 9.38% | 11.10% | 1.20% | 0.00% | 0.00% | 0.00% | 0.00% | 0.16% | 0.00% | 0.14% |
| P4.R3.59 | 7.77% | 14.84% | 1.12% | 0.00% | 0.00% | 0.07% | 0.00% | 0.20% | 0.11% | 0.19% |
| P4.R3.14 | 7.01% | 15.94% | 1.20% | 0.00% | 0.00% | 0.07% | 0.00% | 0.11% | 0.10% | 0.10% |
| P4.R3.47 | 9.53% | 15.56% | 1.13% | 0.00% | 0.00% | 0.00% | 0.00% | 0.13% | 0.10% | 0.12% |
| P4.R3.45 | 8.19% | 14.32% | 0.94% | 0.00% | 0.00% | 0.00% | 0.00% | 0.12% | 0.08% | 0.21% |

| | | | | | | | | Total max |
|---|---|---|---|---|---|---|---|---|
| Variety | | | | | | | TOTAL | Non-CBG |
| Name | CBC | THCV | CBDV | CBGV | CBN | D8-THC | CBG/CBGVmax* | Cannabs* |
| P4.R3.74 | 0.00% | 0.00% | 0.00% | 0.00% | 0.00% | 0.00% | 2.03% | 20.80% |
| P4.R3.43 | 0.00% | 0.00% | 0.00% | 0.00% | 0.00% | 0.00% | 1.93% | 21.96% |
| P4.R3.02 | 0.00% | 0.00% | 0.00% | 0.00% | 0.00% | 0.00% | 1.36% | 20.58% |
| P4.R3.23 | 0.00% | 0.00% | 0.00% | 0.00% | 0.00% | 0.00% | 1.20% | 21.17% |
| P4.R3.34 | 0.00% | 0.00% | 0.00% | 0.00% | 0.00% | 0.00% | 1.19% | 18.11% |
| P4.R3.59 | 0.00% | 0.00% | 0.00% | 0.00% | 0.00% | 0.00% | 1.17% | 20.20% |
| P4.R3.14 | 0.00% | 0.00% | 0.00% | 0.00% | 0.00% | 0.00% | 1.16% | 20.40% |
| P4.R3.47 | 0.00% | 0.00% | 0.00% | 0.00% | 0.00% | 0.00% | 1.11% | 22.23% |
| P4.R3.45 | 0.00% | 0.00% | 0.00% | 0.00% | 0.00% | 0.00% | 1.03% | 19.94% |

*All Max values were calculated based on theoretical maximum formulas disclosed in this application.

TABLE 10
Terpene Contents of GB.OJ high CBG line

| Variety | | alpha | beta | | | gamma | alpha | alpha | beta | | |
|---|---|---|---|---|---|---|---|---|---|---|---|
| Name | terpinolene | phellandrene | ocimene | carene | limonene | terpinene | pinene | terpinene | pinene | fenchol | camphene |
| GB.OJ.28-1 | 0.00% | 0.00% | 0.00% | 0.00% | 0.18% | 0.00% | 0.02% | 0.00% | 0.03% | 0.02% | 0.00% |
| GB.OJ.28-2 | 0.00% | 0.00% | 0.00% | 0.00% | 0.31% | 0.00% | 0.03% | 0.00% | 0.05% | 0.02% | 0.01% |
| GB.OJ.28-3 | 0.00% | 0.00% | 0.00% | 0.00% | 0.23% | 0.00% | 0.03% | 0.00% | 0.05% | 0.03% | 0.00% |
| GB.OJ.28-4 | 0.00% | 0.00% | 0.00% | 0.00% | 0.08% | 0.00% | 0.00% | 0.00% | 0.01% | 0.00% | 0.00% |
| GB.OJ.28-5 | 0.00% | 0.00% | 0.00% | 0.00% | 0.20% | 0.00% | 0.02% | 0.00% | 0.03% | 0.02% | 0.01% |
| GB.OJ.28-6 | 0.00% | 0.00% | 0.00% | 0.00% | 0.22% | 0.00% | 0.02% | 0.00% | 0.04% | 0.02% | 0.01% |
| GB.OJ.28-7 | 0.01% | 0.00% | 0.00% | 0.00% | 0.37% | 0.00% | 0.03% | 0.00% | 0.05% | 0.03% | 0.01% |
| GB.OJ.28-8 | 0.00% | 0.00% | 0.00% | 0.00% | 0.29% | 0.00% | 0.03% | 0.00% | 0.04% | 0.03% | 0.00% |
| GB.OJ.28-9 | 0.00% | 0.00% | 0.00% | 0.00% | 0.17% | 0.00% | 0.01% | 0.00% | 0.02% | 0.02% | 0.00% |

| Variety | alpha | alpha | beta | | caryophyllene | | Terpene Oil |
|---|---|---|---|---|---|---|---|
| Name | terpineol | humulene | caryophyllene | linalool | oxide | myrcene | Content |
| GB.OJ.28-1 | 0.02% | 0.09% | 0.25% | 0.03% | 0.01% | 0.01% | 0.64% |
| GB.OJ.28-2 | 0.03% | 0.15% | 0.44% | 0.04% | 0.01% | 0.02% | 1.10% |
| GB.OJ.28-3 | 0.04% | 0.13% | 0.39% | 0.09% | 0.00% | 0.01% | 1.01% |
| GB.OJ.28-4 | 0.01% | 0.13% | 0.40% | 0.04% | 0.00% | 0.00% | 0.67% |
| GB.OJ.28-5 | 0.02% | 0.21% | 0.47% | 0.04% | 0.01% | 0.01% | 1.03% |
| GB.OJ.28-6 | 0.02% | 0.22% | 0.49% | 0.05% | 0.01% | 0.01% | 1.12% |
| GB.OJ.28-7 | 0.03% | 0.23% | 0.44% | 0.07% | 0.01% | 0.02% | 1.31% |
| GB.OJ.28-8 | 0.03% | 0.23% | 0.55% | 0.04% | 0.01% | 0.02% | 1.26% |
| GB.OJ.28-9 | 0.01% | 0.32% | 0.66% | 0.05% | 0.01% | 0.01% | 1.30% |

Bolded values indicate dominant terpene.

TABLE 11
Terpene Contents of R2.R3 high CBG line.

| Variety | | alpha | beta | | | gamma | alpha | alpha | beta | | |
|---|---|---|---|---|---|---|---|---|---|---|---|
| Name | terpinolene | phellandrene | ocimene | carene | limonene | terpinene | pinene | terpinene | pinene | fenchol | camphene |
| R2.R3.24 | 0.00% | 0.00% | 0.39% | 0.00% | 0.13% | 0.00% | 0.20% | 0.00% | 0.07% | 0.05% | 0.01% |
| R2.R3.47 | 0.00% | 0.00% | 0.16% | 0.00% | 0.06% | 0.00% | 0.16% | 0.00% | 0.05% | 0.03% | 0.01% |
| R2.R3.08 | 0.00% | 0.00% | 0.32% | 0.00% | 0.14% | 0.00% | 0.25% | 0.00% | 0.09% | 0.05% | 0.01% |
| R2.R3.51 | 0.01% | 0.00% | 0.97% | 0.00% | 0.53% | 0.00% | 0.25% | 0.00% | 0.12% | 0.07% | 0.02% |
| R2.R3.05 | 0.00% | 0.00% | 0.11% | 0.00% | 0.09% | 0.00% | 0.11% | 0.00% | 0.06% | 0.05% | 0.01% |
| R2.R3.15 | 0.00% | 0.00% | 0.28% | 0.00% | 0.10% | 0.00% | 0.19% | 0.00% | 0.07% | 0.04% | 0.01% |
| R2.R3.70 | 0.00% | 0.00% | 0.47% | 0.00% | 0.13% | 0.00% | 0.21% | 0.00% | 0.06% | 0.02% | 0.01% |
| R2.R3.16 | 0.00% | 0.00% | 0.44% | 0.00% | 0.16% | 0.00% | 0.18% | 0.00% | 0.06% | 0.04% | 0.01% |
| R2.R3.61 | 0.00% | 0.00% | 0.92% | 0.00% | 0.33% | 0.00% | 0.27% | 0.00% | 0.10% | 0.05% | 0.01% |
| R2.R3.55 | 0.00% | 0.00% | 0.44% | 0.00% | 0.28% | 0.00% | 0.21% | 0.00% | 0.09% | 0.04% | 0.01% |
| R2.R3.49 | 0.00% | 0.00% | 0.46% | 0.00% | 0.25% | 0.00% | 0.33% | 0.00% | 0.13% | 0.07% | 0.02% |
| R2.R3.06 | 0.00% | 0.00% | 0.15% | 0.00% | 0.13% | 0.00% | 0.21% | 0.00% | 0.08% | 0.05% | 0.01% |
| R2.R3.66 | 0.00% | 0.00% | 1.08% | 0.00% | 0.25% | 0.00% | 0.54% | 0.00% | 0.14% | 0.03% | 0.01% |
| R2.R3.64 | 0.00% | 0.00% | 0.41% | 0.00% | 0.17% | 0.00% | 0.22% | 0.00% | 0.08% | 0.05% | 0.01% |
| R2.R3.42 | 0.00% | 0.00% | 0.95% | 0.00% | 0.31% | 0.00% | 0.35% | 0.00% | 0.12% | 0.05% | 0.01% |
| R2.R3.56 | 0.00% | 0.00% | 0.15% | 0.00% | 0.12% | 0.00% | 0.15% | 0.00% | 0.07% | 0.05% | 0.01% |
| R2.R3.60 | 0.00% | 0.00% | 0.35% | 0.00% | 0.24% | 0.00% | 0.24% | 0.00% | 0.10% | 0.06% | 0.01% |
| R2.R3.62 | 0.00% | 0.00% | 0.74% | 0.00% | 0.39% | 0.00% | 0.27% | 0.00% | 0.10% | 0.04% | 0.01% |
| R2.R3.39 | 0.00% | 0.00% | 0.29% | 0.00% | 0.20% | 0.00% | 0.18% | 0.00% | 0.09% | 0.05% | 0.01% |
| R2.R3.41 | 0.00% | 0.00% | 0.23% | 0.00% | 0.12% | 0.00% | 0.17% | 0.00% | 0.06% | 0.04% | 0.01% |
| R2.R3.02 | 0.00% | 0.00% | 0.58% | 0.00% | 0.22% | 0.00% | 0.20% | 0.00% | 0.09% | 0.04% | 0.01% |
| R2.R3.40 | 0.00% | 0.00% | 0.38% | 0.00% | 0.26% | 0.00% | 0.18% | 0.00% | 0.10% | 0.06% | 0.01% |
| R2.R3.21 | 0.00% | 0.00% | 0.34% | 0.00% | 0.16% | 0.00% | 0.28% | 0.00% | 0.09% | 0.04% | 0.01% |
| R2.R3.01 | 0.00% | 0.00% | 0.44% | 0.00% | 0.25% | 0.00% | 0.16% | 0.00% | 0.09% | 0.06% | 0.01% |
| R2.R3.71 | 0.00% | 0.00% | 0.15% | 0.00% | 0.09% | 0.00% | 0.23% | 0.00% | 0.08% | 0.04% | 0.01% |
| R2.R3.45 | 0.00% | 0.00% | 0.35% | 0.00% | 0.24% | 0.00% | 0.17% | 0.00% | 0.09% | 0.05% | 0.01% |
| R2.R3.30 | 0.00% | 0.00% | 0.16% | 0.00% | 0.09% | 0.00% | 0.10% | 0.00% | 0.05% | 0.04% | 0.01% |
| R2.R3.54 | 0.00% | 0.00% | 0.82% | 0.00% | 0.40% | 0.00% | 0.26% | 0.00% | 0.12% | 0.05% | 0.01% |
| R2.R3.28 | 0.01% | 0.00% | 1.04% | 0.00% | 0.51% | 0.00% | 0.36% | 0.00% | 0.16% | 0.06% | 0.02% |

| Variety | alpha | alpha | beta | | caryophyllene | | Terpene Oil |
|---|---|---|---|---|---|---|---|
| Name | terpineol | humulene | caryophyllene | linalool | oxide | myrcene | Content |
| R2.R3.24 | 0.04% | 0.08% | 0.21% | 0.05% | 0.01% | 0.02% | 1.25% |
| R2.R3.47 | 0.02% | 0.06% | 0.16% | 0.03% | 0.00% | 0.01% | 0.75% |
| R2.R3.08 | 0.04% | 0.07% | 0.21% | 0.05% | 0.01% | 0.01% | 1.24% |
| R2.R3.51 | 0.06% | 0.09% | 0.23% | 0.10% | 0.00% | 0.06% | 2.51% |
| R2.R3.05 | 0.04% | 0.04% | 0.10% | 0.06% | 0.00% | 0.01% | 0.67% |
| R2.R3.15 | 0.03% | 0.04% | 0.08% | 0.04% | 0.00% | 0.01% | 0.90% |
| R2.R3.70 | 0.02% | 0.04% | 0.12% | 0.03% | 0.00% | 0.02% | 1.14% |
| R2.R3.16 | 0.03% | 0.02% | 0.06% | 0.04% | 0.00% | 0.02% | 1.05% |
| R2.R3.61 | 0.04% | 0.03% | 0.07% | 0.04% | 0.00% | 0.03% | 1.88% |
| R2.R3.55 | 0.03% | 0.05% | 0.13% | 0.05% | 0.00% | 0.03% | 1.35% |
| R2.R3.49 | 0.06% | 0.04% | 0.13% | 0.04% | 0.00% | 0.02% | 1.55% |
| R2.R3.06 | 0.05% | 0.04% | 0.10% | 0.08% | 0.00% | 0.01% | 0.90% |
| R2.R3.66 | 0.03% | 0.04% | 0.11% | 0.04% | 0.00% | 0.04% | 2.32% |
| R2.R3.64 | 0.04% | 0.08% | 0.24% | 0.06% | 0.01% | 0.02% | 1.38% |
| R2.R3.42 | 0.04% | 0.05% | 0.12% | 0.08% | 0.00% | 0.04% | 2.12% |
| R2.R3.56 | 0.04% | 0.05% | 0.13% | 0.04% | 0.01% | 0.01% | 0.82% |
| R2.R3.60 | 0.05% | 0.05% | 0.14% | 0.06% | 0.00% | 0.02% | 1.30% |
| R2.R3.62 | 0.04% | 0.11% | 0.37% | 0.08% | 0.00% | 0.05% | 2.21% |
| R2.R3.39 | 0.05% | 0.06% | 0.15% | 0.05% | 0.00% | 0.02% | 1.15% |
| R2.R3.41 | 0.03% | 0.03% | 0.09% | 0.03% | 0.00% | 0.01% | 0.81% |
| R2.R3.02 | 0.03% | 0.02% | 0.07% | 0.03% | 0.00% | 0.03% | 1.34% |
| R2.R3.40 | 0.05% | 0.06% | 0.13% | 0.08% | 0.00% | 0.02% | 1.34% |
| R2.R3.21 | 0.03% | 0.07% | 0.20% | 0.05% | 0.01% | 0.01% | 1.28% |
| R2.R3.01 | 0.04% | 0.07% | 0.18% | 0.08% | 0.01% | 0.03% | 1.42% |
| R2.R3.71 | 0.03% | 0.03% | 0.08% | 0.03% | 0.01% | 0.01% | 0.78% |
| R2.R3.45 | 0.04% | 0.03% | 0.09% | 0.05% | 0.00% | 0.03% | 1.14% |
| R2.R3.30 | 0.03% | 0.03% | 0.10% | 0.02% | 0.00% | 0.01% | 0.63% |
| R2.R3.54 | 0.05% | 0.15% | 0.35% | 0.09% | 0.01% | 0.05% | 2.34% |
| R2.R3.28 | 0.05% | 0.11% | 0.27% | 0.13% | 0.01% | 0.06% | 2.77% |

Bolded values indicate dominant terpene.

TABLE 12
Terpene Contents of G2.R3 high CBG line

| Variety | | alpha | beta | | | gamma | alpha | alpha | beta | | |
|---|---|---|---|---|---|---|---|---|---|---|---|
| Name | terpinolene | phellandrene | ocimene | carene | limonene | terpinene | pinene | terpinene | pinene | fenchol | camphene |
| G2.R3.25 | 0.00% | 0.00% | 0.87% | 0.00% | 0.36% | 0.00% | 0.35% | 0.00% | 0.12% | 0.06% | 0.02% |
| G2.R3.04 | 0.00% | 0.00% | 0.57% | 0.00% | 0.26% | 0.00% | 0.33% | 0.00% | 0.11% | 0.06% | 0.02% |
| G2.R3.15 | 0.00% | 0.00% | 0.21% | 0.00% | 0.14% | 0.00% | 0.17% | 0.00% | 0.07% | 0.05% | 0.01% |
| G2.R3.11 | 0.00% | 0.00% | 0.18% | 0.00% | 0.11% | 0.00% | 0.15% | 0.00% | 0.06% | 0.04% | 0.01% |
| G2.R3.09 | 0.01% | 0.00% | 0.53% | 0.00% | 0.37% | 0.00% | 0.22% | 0.00% | 0.10% | 0.06% | 0.02% |
| G2.R3.07 | 0.01% | 0.00% | 1.14% | 0.00% | 0.53% | 0.00% | 0.30% | 0.00% | 0.14% | 0.08% | 0.02% |
| G2.R3.18 | 0.00% | 0.00% | 0.80% | 0.00% | 0.40% | 0.00% | 0.21% | 0.00% | 0.12% | 0.08% | 0.02% |
| G2.R3.25 | 0.00% | 0.00% | 0.80% | 0.00% | 0.36% | 0.00% | 0.42% | 0.00% | 0.14% | 0.05% | 0.02% |

| Variety | alpha | alpha | beta | | caryophyllene | | Terpene Oil |
|---|---|---|---|---|---|---|---|
| Name | terpineol | humulene | caryophyllene | linalool | oxide | myrcene | Content |
| G2.R3.25 | 0.06% | 0.14% | 0.46% | 0.15% | 0.02% | 0.17% | 2.76% |
| G2.R3.04 | 0.06% | 0.10% | 0.24% | 0.10% | 0.02% | 0.06% | 1.90% |
| G2.R3.15 | 0.05% | 0.04% | 0.11% | 0.10% | 0.01% | 0.03% | 1.00% |
| G2.R3.11 | 0.04% | 0.04% | 0.12% | 0.08% | 0.00% | 0.07% | 0.90% |
| G2.R3.09 | 0.06% | 0.04% | 0.13% | 0.13% | 0.00% | 0.19% | 1.85% |
| G2.R3.07 | 0.08% | 0.06% | 0.13% | 0.13% | 0.01% | 0.11% | 2.74% |
| G2.R3.18 | 0.07% | 0.05% | 0.13% | 0.13% | 0.02% | 0.11% | 2.13% |
| G2.R3.25 | 0.04% | 0.04% | 0.11% | 0.06% | 0.00% | 0.12% | 2.15% |

Bolded values indicate dominant terpene.

TABLE 13
Terpene Contents of PP.R3 high CBG line

| Variety | | alpha | beta | | | gamma | alpha | alpha | beta | | |
|---|---|---|---|---|---|---|---|---|---|---|---|
| Name | terpinolene | phellandrene | ocimene | carene | limonene | terpinene | pinene | terpinene | pinene | fenchol | camphene |
| PP.R3.08 | 0.00% | 0.00% | 0.05% | 0.00% | 0.34% | 0.00% | 0.04% | 0.00% | 0.06% | 0.06% | 0.01% |
| PP.R3.26 | 0.00% | 0.00% | 1.11% | 0.00%. | 0.38% | 0.00% | 0.25% | 0.00% | 0.10% | 0.05% | 0.02% |
| PP.R3.46 | 0.00% | 0.00% | 0.62% | 0.00% | 0.24% | 0.00% | 0.31% | 0.00% | 0.10% | 0.05% | 0.02% |
| PP.R3.11 | 0.00% | 0.00% | 0.60% | 0.00% | 0.24% | 0.00% | 0.27% | 0.00% | 0.09% | 0.04% | 0.01% |
| PP.R3.33 | 0.00% | 0.00% | 0.11% | 0.00% | 0.46% | 0.00% | 0.05% | 0.00% | 0.07% | 0.06% | 0.01% |
| PP.R3.41 | 0.01% | 0.00% | 0.07% | 0.00% | 0.51% | 0.00% | 0.06% | 0.00% | 0.09% | 0.09% | 0.02% |
| PP.R3.06 | 0.00% | 0.00% | 0.86% | 0.00% | 0.34% | 0.00% | 0.27% | 0.00% | 0.11% | 0.06% | 0.02% |
| PP.R3.25 | 0.01% | 0.00% | 0.94% | 0.00% | 0.48% | 0.00% | 0.39% | 0.00% | 0.14% | 0.06% | 0.02% |
| PP.R3.40 | 0.01% | 0.00% | 1.15% | 0.00% | 0.56% | 0.00% | 0.56% | 0.00% | 0.18% | 0.07% | 0.02% |
| PP.R3.24 | 0.00% | 0.00% | 0.31% | 0.00% | 0.14% | 0.00% | 0.17% | 0.00% | 0.06% | 0.04% | 0.01% |
| PP.R3.03 | 0.00% | 0.00% | 0.66% | 0.00% | 0.30% | 0.00% | 0.26% | 0.00% | 0.09% | 0.05% | 0.01% |
| PP.R3.37 | 0.00% | 0.00% | 0.52% | 0.00% | 0.25% | 0.00% | 0.32% | 0.00% | 0.10% | 0.05% | 0.02% |
| PP.R3.13 | 0.00% | 0.00% | 0.57% | 0.00% | 0.21% | 0.00% | 0.39% | 0.00% | 0.10% | 0.05% | 0.02% |
| PP.R3.07 | 0.00% | 0.00% | 0.82% | 0.00% | 0.35% | 0.00% | 0.37% | 0.00% | 0.12% | 0.05% | 0.02% |
| PP.R3.31 | 0.00% | 0.00% | 0.52% | 0.00%. | 0.30% | 0.00% | 0.33% | 0.00% | 0.11% | 0.05% | 0.02% |
| PP.R3.05 | 0.00% | 0.00% | 0.56% | 0.00% | 0.21% | 0.00% | 0.30% | 0.00% | 0.09% | 0.04% | 0.01% |
| PP.R3.12 | 0.00% | 0.00% | 0.05% | 0.00% | 0.30% | 0.00% | 0.05% | 0.00% | 0.06% | 0.08% | 0.01% |
| PP.R3.04 | 0.00% | 0.00% | 0.68% | 0.00% | 0.30% | 0.00% | 0.26% | 0.00% | 0.10% | 0.06% | 0.02% |
| PP.R3.39 | 0.01% | 0.00% | 0.34% | 0.00% | 0.67% | 0.00% | 0.08% | 0.00% | 0.10% | 0.08% | 0.02% |
| PP.R3.30 | 0.00% | 0.00% | 0.84% | 0.00% | 0.37% | 0.00% | 0.43% | 0.00% | 0.13% | 0.05% | 0.02% |

| Variety | alpha | alpha | beta | | caryophyllene | | Terpene Oil |
|---|---|---|---|---|---|---|---|
| Name | terpineol | humulene | caryophyllene | linalool | oxide | myrcene | Content |
| PP.R3.08 | 0.06% | 0.16% | 0.54% | 0.17% | 0.02% | 0.06% | 1.57% |
| PP.R3.26 | 0.05% | 0.25% | 0.64% | 0.11% | 0.02% | 0.05% | 3.03% |
| PP.R3.46 | 0.05% | 0.10% | 0.30% | 0.12% | 0.02% | 0.03% | 1.97% |
| PP.R3.11 | 0.04% | 0.13% | 0.26% | 0.09% | 0.02% | 0.12% | 1.91% |
| PP.R3.33 | 0.06% | 0.22% | 0.46% | 0.17% | 0.02% | 0.15% | 1.83% |
| PP.R3.41 | 0.08% | 0.28% | 0.69% | 0.13% | 0.03% | 0.12% | 2.17% |
| PP.R3.06 | 0.05% | 0.15% | 0.40% | 0.13% | 0.02% | 0.04% | 2.44% |
| PP.R3.25 | 0.06% | 0.13% | 0.28% | 0.11% | 0.02% | 0.06% | 2.69% |
| PP.R3.40 | 0.06% | 0.12% | 0.22% | 0.10% | 0.01% | 0.08% | 3.13% |
| PP.R3.24 | 0.04% | 0.15% | 0.28% | 0.10% | 0.02% | 0.02% | 1.35% |
| PP.R3.03 | 0.04% | 0.18% | 0.35% | 0.10% | 0.02% | 0.06% | 2.11% |
| PP.R3.37 | 0.05% | 0.12% | 0.27% | 0.10% | 0.02% | 0.05% | 1.85% |
| PP.R3.13 | 0.05% | 0.21% | 0.51% | 0.08% | 0.02% | 0.03% | 2.23% |
| PP.R3.07 | 0.05% | 0.26% | 0.59% | 0.10% | 0.02% | 0.13% | 2.86% |
| PP.R3.31 | 0.05% | 0.16% | 0.39% | 0.11% | 0.03% | 0.05% | 2.11% |
| PP.R3.05 | 0.03% | 0.14% | 0.38% | 0.06% | 0.02% | 0.03% | 1.87% |
| PP.R3.12 | 0.07% | 0.15% | 0.41% | 0.14% | 0.03% | 0.04% | 1.38% |
| PP.R3.04 | 0.05% | 0.14% | 0.25% | 0.12% | 0.02% | 0.03% | 2.01% |
| PP.R3.39 | 0.07% | 0.13% | 0.29% | 0.15% | 0.02% | 0.13% | 2.08% |
| PP.R3.30 | 0.05% | 0.13% | 0.26% | 0.11% | 0.01% | 0.13% | 2.54% |

Bolded values indicate dominant terpene.

TABLE 14
Terpene Contents of P4.R3 high CBG line

| Variety | | alpha | beta | | | gamma | alpha | alpha | beta | | |
|---|---|---|---|---|---|---|---|---|---|---|---|
| Name | terpinolene | phellandrene | ocimene | carene | limonene | terpinene | pinene | terpinene | pinene | fenchol | camphene |
| P4.R3.74 | 0.00% | 0.00% | 0.15% | 0.00% | 0.09% | 0.00% | 0.11% | 0.00% | 0.05% | 0.05% | 0.01% |
| P4.R3.43 | 0.00% | 0.00% | 0.70% | 0.00% | 0.37% | 0.00% | 0.23% | 0.00% | 0.09% | 0.05% | 0.01% |
| P4.R3.02 | 0.00% | 0.00% | 0.46% | 0.00% | 0.30% | 0.00% | 0.18% | 0.00% | 0.09% | 0.06% | 0.01% |
| P4.R3.23 | 0.00% | 0.00% | 0.68% | 0.00% | 0.26% | 0.00% | 0.29% | 0.00% | 0.11% | 0.04% | 0.01% |
| P4.R3.34 | 0.00% | 0.00% | 0.23% | 0.00% | 0.15% | 0.00% | 0.11% | 0.00% | 0.06% | 0.04% | 0.01% |
| P4.R3.59 | 0.00% | 0.00% | 0.43% | 0.00% | 0.36% | 0.00% | 0.15% | 0.00% | 0.09% | 0.05% | 0.01% |
| P4.R3.14 | 0.00% | 0.00% | 0.83% | 0.00% | 0.37% | 0.00% | 0.32% | 0.00% | 0.12% | 0.05% | 0.01% |
| P4.R3.47 | 0.00% | 0.00% | 0.04% | 0.00% | 0.48% | 0.00% | 0.07% | 0.00% | 0.10% | 0.09% | 0.02% |
| P4.R3.45 | 0.00% | 0.00% | 0.56% | 0.00% | 0.36% | 0.00% | 0.23% | 0.00% | 0.10% | 0.04% | 0.01% |

| Variety | alpha | alpha | beta | | caryophyllene | | Terpene Oil |
|---|---|---|---|---|---|---|---|
| Name | terpineol | humulene | caryophyllene | linalool | oxide | myrcene | Content |
| P4.R3.74 | 0.06% | 0.07% | 0.09% | 0.09% | 0.02% | 0.20% | 0.98% |
| P4.R3.43 | 0.04% | 0.10% | 0.12% | 0.16% | 0.01% | 0.03% | 1.91% |
| P4.R3.02 | 0.05% | 0.11% | 0.14% | 0.12% | 0.01% | 0.64% | 2.17% |
| P4.R3.23 | 0.03% | 0.10% | 0.12% | 0.09% | 0.01% | 0.88% | 2.61% |
| P4.R3.34 | 0.03% | 0.05% | 0.06% | 0.07% | 0.01% | 0.35% | 1.17% |
| P4.R3.59 | 0.04% | 0.18% | 0.27% | 0.16% | 0.01% | 1.09% | 2.84% |
| P4.R3.14 | 0.04% | 0.13% | 0.15% | 0.08% | 0.00% | 0.04% | 2.15% |
| P4.R3.47 | 0.08% | 0.13% | 0.13% | 0.21% | 0.01% | 0.03% | 1.39% |
| P4.R3.45 | 0.03% | 0.07% | 0.07% | 0.09% | 0.00% | 0.03% | 1.59% |

Bolded values indicate dominant terpene.

### Example 7. Chemical Analysis of Named Specialty Cannabis

Additional Specialty Cannabis varieties created through the methods described in this specification are disclosed. The following Specialty Cannabis were derived from the high CBG varieties disclosed in this specification, and were selected for their unique predicted physiological and organoleptic experiences. Each selected line was named prior to being submitted for competition in the 2017 Emerald Cup. The cannabinoid and Terpene Profiles for the Named Specialty Cannabis lines were measured as described in Example 1. The level of cannabinoids for each of the high CBG lines was measured by HPLC, and is presented in Tables 15. Terpenes were measured using GC-FID, and are presented as absolute content measurements based on the percent content by weight of dry inflorescences in Table 16.

These Named Specialty Cannabis lines were previously disclosed in U.S. 62/596,561, which is hereby incorporated by reference in its entirety for all purposes. Named Specialty Cannabis “Cupcake” was derived from the GB.OJ family of FIG. 3.

TABLE 15
Cannabinoid Contents of Named Specialty Cannabis

| Variety | | | | | | | |
|---|---|---|---|---|---|---|---|
| Name(s) | THCA | CBDA | CBGA | THCVA | CBDVA | THC | CBD |
| Cupcake | 19.76% | 0.00% | 5.56% | 0.39% | 0.00% | 0.38% | 0.00% |
| Kashmir Fire | 22.97% | 0.00% | 4.59% | 0.24% | 0.00% | 0.81% | 0.00% |
| Gewürztraminer | 23.91% | 0.00% | 4.42% | 0.29% | 0.00% | 0.52% | 0.00% |
| Strawberry Cream/ | 29.58% | 0.00% | 2.24% | 0.28% | 0.00% | 1.34% | 0.00% |
| Strawberry Creek | | | | | | | |

| | | | | | | Totalmax |
|---|---|---|---|---|---|---|
| Variety | | | | | TOTAL | Non-CBG |
| Name(s) | CBG | CBC | THCV | CBN | CBG/CBGVmax | Cannabs* |
| Cupcake | 0.18% | 0.00% | 0.00% | 0.00% | 5.06% | 18.05% |
| Kashmir Fire | 0.14% | 0.11% | 0.00% | 0.00% | 4.17% | 21.28% |
| Gewürztraminer | 0.18% | 0.00% | 0.00% | 0.00% | 4.06% | 21.74% |
| Strawberry Cream/ | 0.23% | 0.00% | 0.00% | 0.08% | 2.20% | 27.61% |
| Strawberry Creek | | | | | | |

*All Max values were calculated based on theoretical maximum formulas disclosed in this application.

TABLE 16
Terpene Contents of Named Specialty Cannabis

| Variety | | alpha | beta | | | gamma | alpha | alpha | beta | | |
|---|---|---|---|---|---|---|---|---|---|---|---|
| Name | terpinolene | phellandrene | ocimene | carene | limonene | terpinene | pinene | terpinene | pinene | fenchol | camphene |
| Cupcake | 0.00% | 0.00% | 0.00% | 0.00% | 0.23% | 0.00% | 0.03% | 0.00% | 0.05% | 0.03% | 0.01% |
| Kashmir Fire | 1.99% | 0.10% | 0.33% | 0.07% | 0.46% | 0.04% | 0.12% | 0.07% | 0.21% | 0.05% | 0.01% |
| Gewürztraminer | 0.00% | 0.00% | 0.10% | 0.00% | 0.09% | 0.00% | 0.10% | 0.00% | 0.06% | 0.02% | 0.00% |
| Strawberry | 1.48% | 0.09% | 0.38% | 0.07% | 0.25% | 0.04% | 0.11% | 0.06% | 0.19% | 0.03% | 0.01% |
| Cream/ | | | | | | | | | | | |
| Strawberry | | | | | | | | | | | |
| Creek | | | | | | | | | | | |

| Variety | alpha | alpha | beta | | caryophyllene | | Terpene Oil |
|---|---|---|---|---|---|---|---|
| Name | terpineol | humulene | caryophyllene | linalool | oxide | myrcene | Content |
| Cupcake | 0.04% | 0.23% | 0.49% | 0.11% | 0.02% | 0.01% | 1.25% |
| Kashmir Fire | 0.08% | 0.09% | 0.21% | 0.09% | 0.00% | 0.87% | 4.79% |
| Gewürztraminer | 0.03% | 0.13% | 0.26% | 0.12% | 0.02% | 0.29% | 1.22% |
| Strawberry | 0.06% | 0.13% | 0.45% | 0.08% | 0.01% | 0.46% | 3.90% |
| Cream/ | | | | | | | |
| Strawberry | | | | | | | |
| Creek | | | | | | | |

Bolded values indicate dominant terpene.

### Example 8. Bulking Seed and Chemical Analysis

In order to complete seed deposits, selected plants from the G2.R3, PP.R3, and P4.R3 were crossed to PP.R3.08. Seed from these crosses has been deposited at the NCMB under deposit numbers 43257, 43261, 43263, and 43264.

Seeds from these deposits were also grown for analysis. A representative number of plant from these deposited lines will be grown and analyzed according to the methods of Example 1, as presented in Example 6. The results will show that the deposited lines exhibit high CBG contents with functional B_{T} and/or B_{D} alleles. Cannabinoid and terpene contents will be provided.

### Example 9. Cannabinoid Compositions

Flowers from several Specialty Cannabis lines of the present disclosure will be removed from the stems and ground in a blender prior to extraction. Ground material will be packed into a CO2 extraction vessel and tightly closed before allowing flow of CO2 to run in increasing pressure until it reaches manufacturer settings. Three fractions will be collected separately. The first two fractions will be cannabinoid enriched fractions, and the last fraction will be a terpene-enriched fraction. The terpene fraction will be immediately analyzed as described in Example 1, and will be stored for later blending.

Cannabinoid containing fractions will be decarboxylated at high temperatures (175 C) for a period of about 90 minutes. In addition to decarboxylating, this process also allows for water removal from samples prior to winterization. In the winterization process, the two cannabinoid fractions will be pooled and incubated with ethanol at −20 C for a period of at least 24 hours for proper separation of fats and waxes. After incubation, samples will be filtered to remove solidified material.

After the filtering procedure is finished, excess ethanol will be removed by a rotary evaporator, and the remainder of the material will be transferred to a round bottom flask for distillation. The cannabinoid and terpene fractions will be separately analyzed according to the methods of Example 1. Once the distillation process is finished, the cannabinoid and terpene samples will be pooled for patient use

### Deposit Information

A deposit of the cannabis varieties of the present invention, including the Specialty Cannabis, and Named Specialty Cannabis disclosed in this specification (including all lines referenced in Tables 3-16, and FIGS. 3-7), is maintained by the Biotech Institute, LLC 5655 Lindero Canyon Road, Suite 226, Westlake Village, Calif. 91362.

In addition, a sample of one or more varieties of this invention (including all lines referenced in Tables 3, and 5-16, and FIGS. 3-7) have—or will be—deposited with an International Depositary Authority as established under the Budapest Treaty according to 37 CFR 1.803(a)(1), at the National Collections of Industrial, Food and Marine Bacteria Ltd. (NCIMB) in Aberdeen Scotland and/or at the National Center for Marine Algae and Microbiota (NCMA) in East Boothbay, Me.

A sample of half-sibling bulked seed from the ‘G2.R3’ line was deposited as NCIMB 43257 on Nov. 9, 2018. A sample of half-sibling bulked seed from the ‘P4.R3’ line was mixed with a sample of half-sibling bulked seed from the ‘R2.R3’ line, and both were deposited as NCIMB 43261 on Nov. 9, 2018. A sample of selfed seed from the ‘PP.R3’ line was deposited as NCIMB 43263 on Nov. 9, 2018. A sample of half-sibling seed from the ‘R2.R3’ line was deposited as NCIMB 43264 on Nov. 9, 2018.

To satisfy the enablement requirements of 35 U.S.C. 112, and to certify that the deposit of the isolated strains (i.e., cannabis varieties) of the present invention meets the criteria set forth in 37 CFR 1.801-1.809 and Manual of Patent Examining Procedure (MPEP) 2402-2411.05, Applicants hereby make the following statements regarding the deposited cannabis varieties:

If the deposit is made under the terms of the Budapest Treaty, the instant invention will be irrevocably and without restriction released to the public upon the granting of a patent.

If the deposit is made not under the terms of the Budapest Treaty, Applicant(s) provides assurance of compliance by following statements:

1. During the pendency of this application, access to the invention will be afforded to the Commissioner upon request;

2. All restrictions on availability to the public will be irrevocably removed upon granting of the patent under conditions specified in 37 CFR 1.808;

3. The deposit will be maintained in a public repository for a period of 30 years or 5 years after the last request or for the effective life of the patent, whichever is longer;

4. A test of the viability of the biological material at the time of deposit will be conducted by the public depository under 37 CFR 1.807; and

5. The deposit will be replaced if it should ever become unavailable.

Access to this deposit will be available during the pendency of this application to persons determined by the Commissioner of Patents and Trademarks to be entitled thereto under 37 C.F.R. § 1.14 and 35 U.S.C. § 122. Upon granting of any claims in this application, all restrictions on the availability to the public of the variety will be irrevocably removed by affording access to a deposit of at least 2,500 seeds of the same variety with the depository.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the non-limiting exemplary methods and materials are described herein.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. Nothing herein is to be construed as an admission that the present invention is not entitled to antedate such publication by virtue of prior invention.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### FURTHER EMBODIMENTS OF THE INVENTION

Other subject matter contemplated by the present disclosure is set out in the following numbered embodiments:

1. A cannabis plant, or an asexual clone of said cannabis plant, or a plant part, tissue, or cell thereof, which is capable of producing a female inflorescence, said inflorescence comprising:
a) a functional B_{T} allele and/or a functioning B_{D} allele
b) a cannabigerol (CBG max) content of at least 2.0% by weight
c) a non-CBG max cannabinoid content of at least 5.0% by weight,

wherein the contents of all cannabinoids are measured by high performance liquid chromatography (HPLC) and calculated based on dry weight of the inflorescence.

1.1 The cannabis plant, or an asexual clone of said cannabis plant, or a plant part, tissue, or cell thereof of embodiment 1, wherein a representative sample of seed producing said plant has been deposited under NCIMB Nos. 43257, 43261, 43263, and 43264.
1.2 The cannabis plant, or an asexual clone of said cannabis plant, or a plant part, tissue, or cell thereof of embodiment 1, wherein samples of seed that produce plants comprising a), b), and c) have been deposited under NCIMB Nos. 43257, 43261, 43263, and 43264.
1.3 The cannabis plant, or an asexual clone of said cannabis plant, or a plant part, tissue, or cell thereof of embodiment 1, wherein samples of seed that produce plants comprising a), b), and c) are obtainable from seed deposited under NCIMB Nos. 43257, 43261, 43263, and 43264.
2. The cannabis plant, or an asexual clone of said cannabis plant, or a plant part, tissue, or cell thereof of any one of embodiments 1-1.3, wherein the plant does not comprise a functional B_{D} allele.
2.1 The cannabis plant, or an asexual clone of said cannabis plant, or a plant part, tissue, or cell thereof of any one of embodiments 1-1.3, wherein the plant does not comprise a functional B_{T} allele.
3. The cannabis plant, or an asexual clone of said cannabis plant, or a plant part, tissue, or cell thereof of any one of embodiments 1-2.1, wherein the inflorescence comprises a terpene oil content greater than about 1.0% by weight;

wherein the terpene oil content is the additive content of terpinolene, alpha phellandrene, beta ocimene, carene, limonene, gamma terpinene, alpha pinene, alpha terpinene, beta pinene, fenchol, camphene, alpha terpineol, alpha humulene, beta caryophyllene, linalool, caryophyllene oxide, and myrcene as measured by Gas Chromatography Flame Ionization Detection (GC-FID) and calculated based on dry weight of the inflorescence.

4. The cannabis plant, or an asexual clone of said cannabis plant, or a plant part, tissue, or cell thereof of embodiment 3, wherein the inflorescence comprises a terpene oil content greater than about 1.5% by weight.
5. The cannabis plant, or an asexual clone of said cannabis plant, or a plant part, tissue, or cell thereof of embodiment 3, wherein the inflorescence comprises a terpene oil content greater than about 2.0% by weight.
6. The cannabis plant, or an asexual clone of said cannabis plant, or a plant part, tissue, or cell thereof of any one of embodiments 1-5, wherein the inflorescence comprises a CBG max content of at least 3% by weight as measured by HPLC and calculated based on dry weight of the inflorescence.
7. The cannabis plant, or an asexual clone of said cannabis plant, or a plant part, tissue, or cell thereof any one of embodiments 1-5, wherein the inflorescence comprises a CBG max content of at least 4% by weight as measured by HPLC and calculated based on dry weight of the inflorescence.
8. The cannabis plant, or an asexual clone of said cannabis plant, or a plant part, tissue, or cell thereof of any one of embodiments 1-5, wherein the inflorescence comprises a CBG max content of at least 5% by weight as measured by HPLC and calculated based on dry weight of the inflorescence.
9. The cannabis plant, or an asexual clone of said cannabis plant, or a plant part, tissue, or cell thereof of any one of embodiments 1-5, wherein the inflorescence comprises a CBG max content of at least 6% by weight as measured by HPLC and calculated based on dry weight of the inflorescence.
10. The cannabis plant, or an asexual clone of said cannabis plant, or a plant part, tissue, or cell thereof of any one of embodiments 1-5, wherein the inflorescence comprises a CBG max content of at least 7% by weight as measured by HPLC and calculated based on dry weight of the inflorescence.
11. The cannabis plant, or an asexual clone of said cannabis plant, or a plant part, tissue, or cell thereof of any one of embodiments 1-5, wherein the inflorescence comprises a CBG max content of at least 8% by weight as measured by HPLC and calculated based on dry weight of the inflorescence.
12. The cannabis plant, or an asexual clone of said cannabis plant, or a plant part, tissue, or cell thereof of any one of embodiments 1-11, wherein the inflorescence comprises a non-CBG max cannabinoid content that is at least 7.5% by weight as measured by HPLC and calculated based on dry weight of the inflorescence.
13. The cannabis plant, or an asexual clone of said cannabis plant, or a plant part, tissue, or cell thereof of embodiments 1-11, wherein the inflorescence comprises a non-CBG max cannabinoid content that is at least 10.0% by weight as measured by HPLC and calculated based on dry weight of the inflorescence.
14. The cannabis plant, or an asexual clone of said cannabis plant, or a plant part, tissue, or cell thereof of any one of embodiments 1-11, wherein the inflorescence comprises a non-CBG max cannabinoid content that is at least 12.5% by weight as measured by HPLC and calculated based on dry weight of the inflorescence.
15. The cannabis plant, or an asexual clone of said cannabis plant, or a plant part, tissue, or cell thereof of any one of embodiments 1-14, wherein the inflorescence comprises a Terpene Profile in which myrcene is not the dominant terpene; wherein the Terpene Profile is defined as terpinolene, alpha phellandrene, beta ocimene, carene, limonene, gamma terpinene, alpha pinene, alpha terpinene, beta pinene, fenchol, camphene, alpha terpineol, alpha humulene, beta caryophyllene, linalool, caryophyllene oxide, and myrcene.
16. The cannabis plant, or an asexual clone of said cannabis plant, or a plant part, tissue, or cell thereof of embodiment 15, wherein the first or second most abundant terpene in the Terpene Profile is terpinolene.
17. The cannabis plant, or an asexual clone of said cannabis plant, or a plant part, tissue, or cell thereof of embodiment 15, wherein the first or second most abundant terpene in the Terpene Profile is alpha phellandrene.
18. The cannabis plant, or an asexual clone of said cannabis plant, or a plant part, tissue, or cell thereof of embodiment 15, wherein the first or second most abundant terpene in the Terpene Profile is carene.
19. The cannabis plant, or an asexual clone of said cannabis plant, or a plant part, tissue, or cell thereof of embodiment 15, wherein the first or second most abundant terpene in the Terpene Profile is limonene.
20. The cannabis plant, or an asexual clone of said cannabis plant, or a plant part, tissue, or cell thereof of embodiment 15, wherein the first or second most abundant terpene in the Terpene Profile is gamma terpinene.
21. The cannabis plant, or an asexual clone of said cannabis plant, or a plant part, tissue, or cell thereof of embodiment 15, wherein the first or second most abundant terpene in the Terpene Profile is alpha pinene.
22. The cannabis plant, or an asexual clone of said cannabis plant, or a plant part, tissue, or cell thereof of embodiment 15, wherein the first or second most abundant terpene in the Terpene Profile is alpha terpinene.
23. The cannabis plant, or an asexual clone of said cannabis plant, or a plant part, tissue, or cell thereof of embodiment 15, wherein the first or second most abundant terpene in the Terpene Profile is beta pinene.
24. The cannabis plant, or an asexual clone of said cannabis plant, or a plant part, tissue, or cell thereof of embodiment 15, wherein the first or second most abundant terpene in the Terpene Profile is fenchol.
25. The cannabis plant, or an asexual clone of said cannabis plant, or a plant part, tissue, or cell thereof of embodiment 15, wherein the first or second most abundant terpene in the Terpene Profile is camphene.
26. The cannabis plant, or an asexual clone of said cannabis plant, or a plant part, tissue, or cell thereof of embodiment 15, wherein the first or second most abundant terpene in the Terpene Profile is alpha terpineol.
27. The cannabis plant, or an asexual clone of said cannabis plant, or a plant part, tissue, or cell thereof of embodiment 15, wherein the first or second most abundant terpene in the Terpene Profile is alpha humulene.
28. The cannabis plant, or an asexual clone of said cannabis plant, or a plant part, tissue, or cell thereof of embodiment 15, wherein the first or second most abundant terpene in the Terpene Profile is beta caryophyllene.
29. The cannabis plant, or an asexual clone of said cannabis plant, or a plant part, tissue, or cell thereof of embodiment 15, wherein the first or second most abundant terpene in the Terpene Profile is linalool.
30. The cannabis plant, or an asexual clone of said cannabis plant, or a plant part, tissue, or cell thereof of embodiment 15, wherein the first or second most abundant terpene in the Terpene Profile is caryophyllene oxide.
31. The cannabis plant, or an asexual clone of said cannabis plant, or a plant part, tissue, or cell thereof of embodiment 15, wherein the first or second most abundant terpene in the Terpene Profile is beta ocimene.
32. The cannabis plant, or an asexual clone of said cannabis plant, or a plant part, tissue, or cell thereof of any one of embodiments 1-14, wherein the inflorescence comprises a Terpene Profile in which myrcene is the first or second most abundant terpene in the Terpene Profile; wherein the Terpene Profile is defined as terpinolene, alpha phellandrene, beta ocimene, carene, limonene, gamma terpinene, alpha pinene, alpha terpinene, beta pinene, fenchol, camphene, alpha terpineol, alpha humulene, beta caryophyllene, linalool, caryophyllene oxide, and myrcene.
33. A cannabis extract from the cannabis plant, plant part, tissue, or cell of any one of embodiments 1-32.
34. The cannabis extract of embodiment 33, wherein said extract is selected from the group consisting of kief, hashish, bubble hash, solvent reduced oils, sludges, e-juice, and tinctures.
35. The cannabis extract of embodiment 33 wherein said extract comprises greater than 25% CBG max content and greater than 30% non-CBG max cannabinoid content as measured by HPLC and based on weight of the extract.
36. A method of breeding cannabis plants with high CBG max and non-CBG max cannabinoid contents, said method comprising:
(i) making a cross between a first cannabis plant of any one of embodiments 1-32, and a second cannabis plant to produce an F1 plant
(ii) harvesting the resulting seed;
(iii) growing said seed; and
(iv) selecting for high CBG max and non-CBG max cannabinoid contents;
   wherein the resulting selected cannabis plant is comprises at least 2.0% CBG max, and at least 5.0% non-CBG max cannabinoid content.
   37. A method of producing cannabis plants cannabis plants high CBG max and non-CBG max cannabinoid contents, said method comprising:
(i) obtaining a cannabis seed, or cutting from a first cannabis plant of any one of embodiments 1-32,
(ii) placing said cannabis seed or cutting in an environment conducive to plant growth;
(iii) allowing said cannabis seed or cutting to produce a cannabis plant;
(iv) selecting for with high CBG max and non-CBG max cannabinoid contents;
   wherein the resulting selected cannabis plant is comprises at least 2.0% CBG max, and at least 5.0% non-CBG max cannabinoid content.
   38. A dry, non-viable (i) cannabis plant or (ii) part thereof, wherein said cannabis hemp plant or part thereof, comprises at least a portion of a female inflorescence, said inflorescence comprising:
a) a functional B_{T} allele
b) a cannabigerol (CBG max) content of at least 2.0% by weight
c) a non-CBG max cannabinoid content of at least 5.0% by weight

wherein the contents of all cannabinoids are measured by high performance liquid chromatography (HPLC) and calculated based on dry weight of the inflorescence.

38.1 The dry, non-viable (i) cannabis plant or (ii) part thereof of embodiment 38, wherein a representative sample of seed producing said inflorescence has been deposited under NCIMB Nos. 43257, 43261, 43263, and 43264.
38.2 The dry, non-viable (i) cannabis plant or (ii) part thereof of embodiment 38, wherein samples of seed that produce inflorescences comprising a), b), and c) have been deposited under NCIMB Nos. 43257, 43261, 43263, and 43264.
38.3 The dry, non-viable (i) cannabis plant or (ii) part thereof of embodiment 38, wherein samples of seed that produce inflorescences comprising a), b), and c) are obtainable from seed deposited under NCIMB Nos. 43257, 43261, 43263, and 43264.
39. The dry, non-viable (i) cannabis plant or (ii) part thereof of any one of embodiments 38-38.3, wherein the inflorescence does not comprise a functional B_{D} allele.
39.1 The dry, non-viable (i) cannabis plant or (ii) part thereof of any one of embodiments 38-38.3, wherein the inflorescence does not comprise a functional Br allele.
40. The dry, non-viable (i) cannabis plant or (ii) part thereof of any one of embodiments 38-39.1, wherein the inflorescence comprises a terpene oil content greater than about 1.0% by weight;

wherein the terpene oil content is the additive content of terpinolene, alpha phellandrene, beta ocimene, carene, limonene, gamma terpinene, alpha pinene, alpha terpinene, beta pinene, fenchol, camphene, alpha terpineol, alpha humulene, beta caryophyllene, linalool, caryophyllene oxide, and myrcene as measured by GC-FID and calculated based on dry weight of the inflorescence.

41. The dry, non-viable (i) cannabis plant or (ii) part thereof of embodiment 40, wherein the inflorescence comprises a terpene oil content greater than about 1.5% by weight.
42. The dry, non-viable (i) cannabis plant or (ii) part thereof of embodiment 40, wherein the inflorescence comprises a terpene oil content greater than about 2.0% by weight.
43. The dry, non-viable (i) cannabis plant or (ii) part thereof of any one of embodiments 38-42, wherein the inflorescence comprises a CBG max content of at least 3% by weight as measured by HPLC and calculated based on dry weight of the inflorescence.
44. The dry, non-viable (i) cannabis plant or (ii) part thereof of any one of embodiments 38-42, wherein the inflorescence comprises a CBG max content of at least 4% by weight as measured by HPLC and calculated based on dry weight of the inflorescence.
45. The dry, non-viable (i) cannabis plant or (ii) part thereof of any one of embodiments 38-42, wherein the inflorescence comprises a CBG max content of at least 5% by weight as measured by HPLC and calculated based on dry weight of the inflorescence.
46. The dry, non-viable (i) cannabis plant or (ii) part thereof of any one of embodiments 38-42, wherein the inflorescence comprises a CBG max content of at least 6% by weight as measured by HPLC and calculated based on dry weight of the inflorescence.
47. The dry, non-viable (i) cannabis plant or (ii) part thereof of any one of embodiments 38-42, wherein the inflorescence comprises a CBG max content of at least 7% by weight as measured by HPLC and calculated based on dry weight of the inflorescence.
48. The dry, non-viable (i) cannabis plant or (ii) part thereof of any one of embodiments 38-42, wherein the inflorescence comprises a CBG max content of at least 8% by weight as measured by HPLC and calculated based on dry weight of the inflorescence.
49. The dry, non-viable (i) cannabis plant or (ii) part thereof of any one of embodiments 38-48, wherein the inflorescence comprises a non-CBG max cannabinoid content that is at least 7.5% by weight as measured by HPLC and calculated based on dry weight of the inflorescence.
50. The dry, non-viable (i) cannabis plant or (ii) part thereof of any one of embodiments 38-48, wherein the inflorescence comprises a non-CBG max cannabinoid content that is at least 10.0% by weight as measured by HPLC and calculated based on dry weight of the inflorescence.
51. The dry, non-viable (i) cannabis plant or (ii) part thereof of any one of embodiments 38-48, wherein the inflorescence comprises a non-CBG max cannabinoid content that is at least 12.5% by weight as measured by HPLC and calculated based on dry weight of the inflorescence.
52. The dry, non-viable (i) cannabis plant or (ii) part thereof of any one of embodiments 38-51, wherein the inflorescence comprises a Terpene Profile in which myrcene is not the dominant terpene; wherein the Terpene Profile is defined as terpinolene, alpha phellandrene, beta ocimene, carene, limonene, gamma terpinene, alpha pinene, alpha terpinene, beta pinene, fenchol, camphene, alpha terpineol, alpha humulene, beta caryophyllene, linalool, caryophyllene oxide, and myrcene.
53. The dry, non-viable (i) cannabis plant or (ii) part thereof of embodiment 52, wherein the first or second most abundant terpene in the Terpene Profile is terpinolene.
54. The dry, non-viable (i) cannabis plant or (ii) part thereof of embodiment 52, wherein the first or second most abundant terpene in the Terpene Profile is alpha phellandrene.
55. The dry, non-viable (i) cannabis plant or (ii) part thereof of embodiment 52, wherein the first or second most abundant terpene in the Terpene Profile is carene.
56. The dry, non-viable (i) cannabis plant or (ii) part thereof of embodiment 52, wherein the first or second most abundant terpene in the Terpene Profile is limonene.
57. The dry, non-viable (i) cannabis plant or (ii) part thereof of embodiment 52, wherein the first or second most abundant terpene in the Terpene Profile is gamma terpinene.
58. The dry, non-viable (i) cannabis plant or (ii) part thereof of embodiment 52, wherein the first or second most abundant terpene in the Terpene Profile is alpha pinene.
59. The dry, non-viable (i) cannabis plant or (ii) part thereof of embodiment 52, wherein the first or second most abundant terpene in the Terpene Profile is alpha terpinene.
60. The dry, non-viable (i) cannabis plant or (ii) part thereof of embodiment 52, wherein the first or second most abundant terpene in the Terpene Profile is beta pinene.
61. The dry, non-viable (i) cannabis plant or (ii) part thereof of embodiment 52, wherein the first or second most abundant terpene in the Terpene Profile is fenchol.
62. The dry, non-viable (i) cannabis plant or (ii) part thereof of embodiment 52, wherein the first or second most abundant terpene in the Terpene Profile is camphene.
63. The dry, non-viable (i) cannabis plant or (ii) part thereof of embodiment 52, wherein the first or second most abundant terpene in the Terpene Profile is alpha terpineol.
64. The dry, non-viable (i) cannabis plant or (ii) part thereof of embodiment 52, wherein the first or second most abundant terpene in the Terpene Profile is alpha humulene.
65. The dry, non-viable (i) cannabis plant or (ii) part thereof of embodiment 52, wherein the first or second most abundant terpene in the Terpene Profile is beta caryophyllene.
66. The dry, non-viable (i) cannabis plant or (ii) part thereof of embodiment 52, wherein the first or second most abundant terpene in the Terpene Profile is linalool.
67. The dry, non-viable (i) cannabis plant or (ii) part thereof of embodiment 52, wherein the first or second most abundant terpene in the Terpene Profile is caryophyllene oxide.
68. The dry, non-viable (i) cannabis plant or (ii) part thereof of embodiment 52, wherein the first or second most abundant terpene in the Terpene Profile is beta ocimene.
69. The dry, non-viable (i) cannabis plant or (ii) part thereof of any one of embodiments 38-51, wherein the inflorescence comprises a Terpene Profile in which myrcene is the first or second most abundant terpene in the Terpene Profile; wherein the Terpene Profile is defined as terpinolene, alpha phellandrene, beta ocimene, carene, limonene, gamma terpinene, alpha pinene, alpha terpinene, beta pinene, fenchol, camphene, alpha terpineol, alpha humulene, beta caryophyllene, linalool, caryophyllene oxide, and myrcene.
70. A cannabis extract from the cannabis plant, plant part, tissue, or cell of any one of embodiments 38-69.
71. The cannabis extract of embodiment 70, wherein said extract is selected from the group consisting of kief, hashish, bubble hash, solvent reduced oils, sludges, e-juice, and tinctures.
72. The cannabis extract of embodiment 70 wherein said extract comprises greater than 25% CBG max content and greater than 30% non-CBG max cannabinoid content as measured by HPLC and based on weight of the extract.
73. A cannabis plant, or an asexual clone of said cannabis plant, or a plant part, tissue, or cell thereof, which is capable of producing a female inflorescence, said inflorescence comprising:
a) a cannabigerol (CBG max) content of at least 2.0% by weight
b) a tetrahydrocannabinol (THC max) and cannabidiol (CBD max) combined content of at least 5.0%,

wherein the contents of all cannabinoids are measured by high performance liquid chromatography (HPLC) and calculated based on dry weight of the inflorescence.

73.1 The cannabis plant, or an asexual clone of said cannabis plant, or a plant part, tissue, or cell thereof of embodiment 73, wherein a representative sample of seed producing said plant has been deposited under NCIMB Nos. 43257, 43261, 43263, and 43264.
73.2 The cannabis plant, or an asexual clone of said cannabis plant, or a plant part, tissue, or cell thereof of embodiment 73, wherein samples of seed that produce plants comprising a), and b) have been deposited under NCIMB Nos. 43257, 43261, 43263, and 43264.
73.3 The cannabis plant, or an asexual clone of said cannabis plant, or a plant part, tissue, or cell thereof of embodiment 73, wherein samples of seed that produce plants comprising a), and b) are obtainable from seed deposited under NCIMB Nos. 43257, 43261, 43263, and 43264.
74. The cannabis plant, or an asexual clone of said cannabis plant, or a plant part, tissue, or cell thereof of any one of embodiments 73-73.3, wherein the inflorescence comprises less than 1% CBD max.
75. The cannabis plant, or an asexual clone of said cannabis plant, or a plant part, tissue, or cell thereof of any one of embodiments 73-74, wherein the inflorescence comprises a combined terpene oil content of terpinolene, alpha phellandrene, beta ocimene, carene, limonene, gamma terpinene, alpha pinene, alpha terpinene, beta pinene, fenchol, camphene, alpha terpineol, alpha humulene, beta caryophyllene, linalool, caryophyllene oxide, and myrcene of at least 1.0%, as measured by GC-FID and calculated based on dry weight of the inflorescence.
76. The cannabis plant, or an asexual clone of said cannabis plant, or a plant part, tissue, or cell thereof of embodiment 75, wherein the inflorescence comprises a combined terpene oil content greater than about 1.5% by weight.
77. The cannabis plant, or an asexual clone of said cannabis plant, or a plant part, tissue, or cell thereof of embodiment 75, wherein the inflorescence comprises a combined terpene oil content greater than about 2.0% by weight.
78. The cannabis plant, or an asexual clone of said cannabis plant, or a plant part, tissue, or cell thereof of any one of embodiments 73-77, wherein the inflorescence comprises a CBG max content of at least 3% by weight as measured by HPLC and calculated based on dry weight of the inflorescence.
79. The cannabis plant, or an asexual clone of said cannabis plant, or a plant part, tissue, or cell thereof of any one of embodiments 73-77, wherein the inflorescence comprises a CBG max content of at least 4% by weight as measured by HPLC and calculated based on dry weight of the inflorescence.
80. The cannabis plant, or an asexual clone of said cannabis plant, or a plant part, tissue, or cell thereof of any one of embodiments 73-77, wherein the inflorescence comprises a CBG max content of at least 5% by weight as measured by HPLC and calculated based on dry weight of the inflorescence.
81. The cannabis plant, or an asexual clone of said cannabis plant, or a plant part, tissue, or cell thereof of any one of embodiments 73-80, wherein the tetrahydrocannabinol (THC max) and cannabidiol (CBD max) combined content is at least 7.5% by weight as measured by HPLC and calculated based on dry weight of the inflorescence.
82. The cannabis plant, or an asexual clone of said cannabis plant, or a plant part, tissue, or cell thereof of any one of embodiments 73-80, wherein the tetrahydrocannabinol (THC max) and cannabidiol (CBD max) combined content is at least 10.0% by weight as measured by HPLC and calculated based on dry weight of the inflorescence.
83. The cannabis plant, or an asexual clone of said cannabis plant, or a plant part, tissue, or cell thereof of any one of embodiments 73-80, wherein the tetrahydrocannabinol (THC max) and cannabidiol (CBD max) combined content is at least 12.5% by weight as measured by HPLC and calculated based on dry weight of the inflorescence.
84. A composition comprising:
a) a cannabigerol (CBG max) content of at least 20% by weight, and
b) a non-CBG max cannabinoid content of at least 30.0% by weight,

wherein the contents of all cannabinoids are measured by high performance liquid chromatography (HPLC) and calculated based on weight of the composition.

85. The composition of embodiment 84, wherein the composition comprises a terpene oil content greater than about 5% by weight;

wherein the terpene oil content is the additive content of terpinolene, alpha phellandrene, beta ocimene, carene, limonene, gamma terpinene, alpha pinene, alpha terpinene, beta pinene, fenchol, camphene, alpha terpineol, alpha humulene, beta caryophyllene, linalool, caryophyllene oxide, and myrcene as measured by GC-FID and calculated based on weight of the composition.

86. The composition of embodiment 84 or 85, wherein the composition comprises a terpene oil content greater than about 8% by weight.
87. The composition of embodiment 84 or 85, wherein the composition comprises a terpene oil content greater than about 25.0% by weight.
88. The composition of any one of embodiments 84-87, wherein the composition comprises a CBG max content of at least 30% by weight as measured by HPLC and calculated based on weight of the composition.
89. The composition of any one of embodiments 84-87, wherein the composition comprises a CBG max content of at least 40% by weight as measured by HPLC and calculated based on weight of the composition.
90. The composition of any one of embodiments 84-87, wherein the composition comprises a CBG max content of at least 50% by weight as measured by HPLC and calculated based on dry of the composition.
91. The composition of any one of embodiments 84-87, wherein the composition comprises a CBG max content of at least 60% by weight as measured by HPLC and calculated based on dry weight of the composition.
92. The composition of any one of embodiments 84-87, wherein the composition comprises a CBG max content of at least 70% by weight as measured by HPLC and calculated based on dry weight of the composition.

1. A cannabis plant, or an asexual clone of said cannabis plant, or a plant part, tissue, or cell thereof, which is capable of producing a female inflorescence, said inflorescence comprising:
a) a functional B_{T} allele and/or a functioning B_{D} allele
b) a cannabigerol (CBG max) content of at least 2.0% by weight
c) a non-CBG max cannabinoid content of at least 5.0% by weight,
wherein the contents of all cannabinoids are measured by high performance liquid chromatography (HPLC) and calculated based on dry weight of the inflorescence; wherein a representative sample of seed producing said plant has been deposited under NCIMB Nos. 43257, 43261, 43263, and 43264.

2. The cannabis plant, or an asexual clone of said cannabis plant, or a plant part, tissue, or cell thereof of claim 1, wherein the plant does not comprise a functional B_{D} allele.

3. The cannabis plant, or an asexual clone of said cannabis plant, or a plant part, tissue, or cell thereof of claim 1, wherein the inflorescence comprises a terpene oil content greater than about 1.0% by weight;
wherein the terpene oil content is the additive content of terpinolene, alpha phellandrene, beta ocimene, carene, limonene, gamma terpinene, alpha pinene, alpha terpinene, beta pinene, fenchol, camphene, alpha terpineol, alpha humulene, beta caryophyllene, linalool, caryophyllene oxide, and myrcene as measured by GC-FID and calculated based on dry weight of the inflorescence.

4. The cannabis plant, or an asexual clone of said cannabis plant, or a plant part, tissue, or cell thereof of claim 3, wherein the inflorescence comprises a terpene oil content greater than about 1.5% by weight.

5. The cannabis plant, or an asexual clone of said cannabis plant, or a plant part, tissue, or cell thereof of claim 3, wherein the inflorescence comprises a terpene oil content greater than about 2.0% by weight.

6. The cannabis plant, or an asexual clone of said cannabis plant, or a plant part, tissue, or cell thereof of claim 2, wherein the inflorescence comprises a CBG max content of at least 3% by weight as measured by HPLC and calculated based on dry weight of the inflorescence.

7. The cannabis plant, or an asexual clone of said cannabis plant, or a plant part, tissue, or cell thereof of claim 2, wherein the inflorescence comprises a CBG max content of at least 4% by weight as measured by HPLC and calculated based on dry weight of the inflorescence.

8. The cannabis plant, or an asexual clone of said cannabis plant, or a plant part, tissue, or cell thereof of claim 2, wherein the inflorescence comprises a CBG max content of at least 5% by weight as measured by HPLC and calculated based on dry weight of the inflorescence.

9. The cannabis plant, or an asexual clone of said cannabis plant, or a plant part, tissue, or cell thereof of claim 2, wherein the inflorescence comprises a CBG max content of at least 6% by weight as measured by HPLC and calculated based on dry weight of the inflorescence.

10. The cannabis plant, or an asexual clone of said cannabis plant, or a plant part, tissue, or cell thereof of claim 2, wherein the inflorescence comprises a CBG max content of at least 7% by weight as measured by HPLC and calculated based on dry weight of the inflorescence.

11. The cannabis plant, or an asexual clone of said cannabis plant, or a plant part, tissue, or cell thereof of claim 2, wherein the inflorescence comprises a CBG max content of at least 8% by weight as measured by HPLC and calculated based on dry weight of the inflorescence.

12. The cannabis plant, or an asexual clone of said cannabis plant, or a plant part, tissue, or cell thereof of claim 1, wherein the inflorescence comprises a non-CBG max cannabinoid content that is at least 7.5% by weight as measured by HPLC and calculated based on dry weight of the inflorescence.

13. The cannabis plant, or an asexual clone of said cannabis plant, or a plant part, tissue, or cell thereof of claim 1, wherein the inflorescence comprises a non-CBG max cannabinoid content that is at least 10.0% by weight as measured by HPLC and calculated based on dry weight of the inflorescence.

14. The cannabis plant, or an asexual clone of said cannabis plant, or a plant part, tissue, or cell thereof of claim 1, wherein the inflorescence comprises a non-CBG max cannabinoid content that is at least 12.5% by weight as measured by HPLC and calculated based on dry weight of the inflorescence.

15. The cannabis plant, or an asexual clone of said cannabis plant, or a plant part, tissue, or cell thereof of claim 1, wherein the inflorescence comprises a Terpene Profile in which myrcene is not the dominant terpene; wherein the Terpene Profile is defined as terpinolene, alpha phellandrene, beta ocimene, carene, limonene, gamma terpinene, alpha pinene, alpha terpinene, beta pinene, fenchol, camphene, alpha terpineol, alpha humulene, beta caryophyllene, linalool, caryophyllene oxide, and myrcene.

16. The cannabis plant, or an asexual clone of said cannabis plant, or a plant part, tissue, or cell thereof of claim 15, wherein the first or second most abundant terpene in the Terpene Profile is terpinolene.

17. The cannabis plant, or an asexual clone of said cannabis plant, or a plant part, tissue, or cell thereof of claim 15, wherein the first or second most abundant terpene in the Terpene Profile is alpha phellandrene.

18. The cannabis plant, or an asexual clone of said cannabis plant, or a plant part, tissue, or cell thereof of claim 15, wherein the first or second most abundant terpene in the Terpene Profile is carene.

19. The cannabis plant, or an asexual clone of said cannabis plant, or a plant part, tissue, or cell thereof of claim 15, wherein the first or second most abundant terpene in the Terpene Profile is limonene.

20. The cannabis plant, or an asexual clone of said cannabis plant, or a plant part, tissue, or cell thereof of claim 15, wherein the first or second most abundant terpene in the Terpene Profile is gamma terpinene.

21. The cannabis plant, or an asexual clone of said cannabis plant, or a plant part, tissue, or cell thereof of claim 15, wherein the first or second most abundant terpene in the Terpene Profile is alpha pinene.

22. The cannabis plant, or an asexual clone of said cannabis plant, or a plant part, tissue, or cell thereof of claim 15, wherein the first or second most abundant terpene in the Terpene Profile is alpha terpinene.

23. The cannabis plant, or an asexual clone of said cannabis plant, or a plant part, tissue, or cell thereof of claim 15, wherein the first or second most abundant terpene in the Terpene Profile is beta pinene.

24. The cannabis plant, or an asexual clone of said cannabis plant, or a plant part, tissue, or cell thereof of claim 15, wherein the first or second most abundant terpene in the Terpene Profile is fenchol.

25. The cannabis plant, or an asexual clone of said cannabis plant, or a plant part, tissue, or cell thereof of claim 15, wherein the first or second most abundant terpene in the Terpene Profile is camphene.

26. The cannabis plant, or an asexual clone of said cannabis plant, or a plant part, tissue, or cell thereof of claim 15, wherein the first or second most abundant terpene in the Terpene Profile is alpha terpineol.

27. The cannabis plant, or an asexual clone of said cannabis plant, or a plant part, tissue, or cell thereof of claim 15, wherein the first or second most abundant terpene in the Terpene Profile is alpha humulene.

28. The cannabis plant, or an asexual clone of said cannabis plant, or a plant part, tissue, or cell thereof of claim 15, wherein the first or second most abundant terpene in the Terpene Profile is beta caryophyllene.

29. The cannabis plant, or an asexual clone of said cannabis plant, or a plant part, tissue, or cell thereof of claim 15, wherein the first or second most abundant terpene in the Terpene Profile is linalool.

30. The cannabis plant, or an asexual clone of said cannabis plant, or a plant part, tissue, or cell thereof of claim 15, wherein the first or second most abundant terpene in the Terpene Profile is caryophyllene oxide.

31. The cannabis plant, or an asexual clone of said cannabis plant, or a plant part, tissue, or cell thereof of claim 15, wherein the first or second most abundant terpene in the Terpene Profile is beta ocimene.

32. The cannabis plant, or an asexual clone of said cannabis plant, or a plant part, tissue, or cell thereof of claim 15, wherein the first or second most abundant terpene in the Terpene Profile is myrcene.

33. A cannabis extract from the cannabis plant, plant part, tissue, or cell of any one of claims 1-32.

34. The cannabis extract of claim 33, wherein said extract is selected from the group consisting of kief, hashish, bubble hash, solvent reduced oils, sludges, e-juice, and tinctures.

35. The cannabis extract of claim 33, wherein said extract comprises greater than 25% CBG max content and greater than 10% non-CBG max cannabinoid content as measured by HPLC and based on weight of the extract.

36. A method of breeding cannabis plants with high CBG max and non-CBG max cannabinoid contents, said method comprising:
(i) making a cross between a first cannabis plant of claim 1, and a second cannabis plant to produce an F1 plant
(ii) harvesting the resulting seed;
(iii) growing said seed; and
(iv) selecting for the desired phenotypes;
wherein the resulting selected cannabis plant is comprises at least 2.0% CBG max, and at least 5.0% non-CBG max cannabinoid content.

37. A method of producing cannabis plants cannabis plants high CBG max and non-CBG max cannabinoid contents, said method comprising:
(i) obtaining a cannabis seed, or cutting from a first cannabis plant of claim 1,
(ii) placing said cannabis seed or cutting in an environment conducive to plant growth;
(iii) allowing said cannabis seed or cutting to produce a cannabis plant;
(iv) selecting for the desired phenotypes;
wherein the resulting selected cannabis plant is comprises at least 2.0% CBG max, and at least 5.0% non-CBG max cannabinoid content.

38. A cannabis female inflorescence comprising:
a) a functional B_{T} allele
b) a cannabigerol (CBG max) content of at least 2.0% by weight
c) a non-CBG max cannabinoid content of at least 5.0% by weight
wherein the contents of all cannabinoids are measured by high performance liquid chromatography (HPLC) and calculated based on dry weight of the inflorescence; wherein a representative sample of seed producing said inflorescence has been deposited under NCIMB Nos. 43257, 43261, 43263, and 43264.

39. The cannabis female inflorescence of claim 38, wherein the inflorescence does not comprise a functional B_{D} allele.

40. The cannabis female inflorescence of claim 38, wherein the inflorescence comprises a terpene oil content greater than about 1.0% by weight;
wherein the terpene oil content is the additive content of terpinolene, alpha phellandrene, beta ocimene, carene, limonene, gamma terpinene, alpha pinene, alpha terpinene, beta pinene, fenchol, camphene, alpha terpineol, alpha humulene, beta caryophyllene, linalool, caryophyllene oxide, and myrcene as measured by GC-FID and calculated based on dry weight of the inflorescence.

41. The cannabis female inflorescence of claim 38, wherein the inflorescence comprises a terpene oil content greater than about 1.5% by weight.

42. The cannabis female inflorescence of claim 38, wherein the inflorescence comprises a terpene oil content greater than about 2.0% by weight.

43. The cannabis female inflorescence of claim 39, wherein the inflorescence comprises a CBG max content of at least 3% by weight as measured by HPLC and calculated based on dry weight of the inflorescence.

44. The cannabis female inflorescence of claim 39, wherein the inflorescence comprises a CBG max content of at least 4% by weight as measured by HPLC and calculated based on dry weight of the inflorescence.

45. The cannabis female inflorescence of claim 39, wherein the inflorescence comprises a CBG max content of at least 5% by weight as measured by HPLC and calculated based on dry weight of the inflorescence.

46. The cannabis female inflorescence of claim 39, wherein the inflorescence comprises a CBG max content of at least 6% by weight as measured by HPLC and calculated based on dry weight of the inflorescence.

47. The cannabis female inflorescence of claim 39, wherein the inflorescence comprises a CBG max content of at least 7% by weight as measured by HPLC and calculated based on dry weight of the inflorescence.

48. The cannabis female inflorescence of claim 39, wherein the inflorescence comprises a CBG max content of at least 8% by weight as measured by HPLC and calculated based on dry weight of the inflorescence.

49. The cannabis female inflorescence of claim 38, wherein the inflorescence comprises a non-CBG max cannabinoid content that is at least 7.5% by weight as measured by HPLC and calculated based on dry weight of the inflorescence.

50. The cannabis female inflorescence of claim 38, wherein the inflorescence comprises a non-CBG max cannabinoid content that is at least 10.0% by weight as measured by HPLC and calculated based on dry weight of the inflorescence.

51. The cannabis female inflorescence of claim 38, wherein the inflorescence comprises a non-CBG max cannabinoid content that is at least 12.5% by weight as measured by HPLC and calculated based on dry weight of the inflorescence.

52. The cannabis female inflorescence of claim 38, wherein the inflorescence comprises a Terpene Profile in which myrcene is not the dominant terpene; wherein the Terpene Profile is defined as terpinolene, alpha phellandrene, beta ocimene, carene, limonene, gamma terpinene, alpha pinene, alpha terpinene, beta pinene, fenchol, camphene, alpha terpineol, alpha humulene, beta caryophyllene, linalool, caryophyllene oxide, and myrcene.

53. The cannabis female inflorescence of claim 52, wherein the first or second most abundant terpene in the Terpene Profile is terpinolene.

54. The cannabis female inflorescence of claim 52, wherein the first or second most abundant terpene in the Terpene Profile is alpha phellandrene.

55. The cannabis female inflorescence of claim 52, wherein the first or second most abundant terpene in the Terpene Profile is carene.

56. The cannabis female inflorescence of claim 52, wherein the first or second most abundant terpene in the Terpene Profile is limonene.

57. The cannabis female inflorescence of claim 52, wherein the first or second most abundant terpene in the Terpene Profile is gamma terpinene.

58. The cannabis female inflorescence of claim 52, wherein the first or second most abundant terpene in the Terpene Profile is alpha pinene.

59. The cannabis female inflorescence of claim 52, wherein the first or second most abundant terpene in the Terpene Profile is alpha terpinene.

60. The cannabis female inflorescence of claim 52, wherein the first or second most abundant terpene in the Terpene Profile is beta pinene.

61. The cannabis female inflorescence of claim 52, wherein the first or second most abundant terpene in the Terpene Profile is fenchol.

62. The cannabis female inflorescence of claim 52, wherein the first or second most abundant terpene in the Terpene Profile is camphene.

63. The cannabis female inflorescence of claim 52, wherein the first or second most abundant terpene in the Terpene Profile is alpha terpineol.

64. The cannabis female inflorescence of claim 52, wherein the first or second most abundant terpene in the Terpene Profile is alpha humulene.

65. The cannabis female inflorescence of claim 52, wherein the first or second most abundant terpene in the Terpene Profile is beta caryophyllene.

66. The cannabis female inflorescence of claim 52, wherein the first or second most abundant terpene in the Terpene Profile is linalool.

67. The cannabis female inflorescence of claim 52, wherein the first or second most abundant terpene in the Terpene Profile is caryophyllene oxide.

68. The cannabis female inflorescence of claim 52, wherein the first or second most abundant terpene in the Terpene Profile is beta ocimene.

69. The cannabis female inflorescence of claim 52, wherein the first or second most abundant terpene in the Terpene Profile is myrcene.

70. A cannabis extract from the cannabis plant, plant part, tissue, or cell of any one of claims 38-69.

71. The cannabis extract of claim 70, wherein said extract is selected from the group consisting of kief, hashish, bubble hash, solvent reduced oils, sludges, e-juice, and tinctures.

72. The cannabis extract of claim 70, wherein said extract comprises greater than 25% CBG max content and greater than 10% non-CBG max cannabinoid content as measured by HPLC and based on weight of the extract.

73. A cannabis plant, or an asexual clone of said cannabis plant, or a plant part, tissue, or cell thereof, which is capable of producing a female inflorescence, said inflorescence comprising:
a) a cannabigerol (CBG max) content of at least 2.0% by weight
b) a tetrahydrocannabinol (THC max) and cannabidiol (CBD max) combined content of at least 5.0%,
wherein the contents of all cannabinoids are measured by high performance liquid chromatography (HPLC) and calculated based on dry weight of the inflorescence; wherein a representative sample of seed producing said plant has been deposited under NCIMB Nos. 43257, 43261, 43263, and 43264.

74. The cannabis plant, or an asexual clone of said cannabis plant, or a plant part, tissue, or cell thereof of claim 73, wherein the inflorescence comprises less than 1% CBD max.

75. The cannabis plant, or an asexual clone of said cannabis plant, or a plant part, tissue, or cell thereof of claim 73, wherein the inflorescence comprises a combined terpene oil content of terpinolene, alpha phellandrene, beta ocimene, carene, limonene, gamma terpinene, alpha pinene, alpha terpinene, beta pinene, fenchol, camphene, alpha terpineol, alpha humulene, beta caryophyllene, linalool, caryophyllene oxide, and myrcene of at least 1.0%, as measured by GC-FID and calculated based on dry weight of the inflorescence.

76. The cannabis plant, or an asexual clone of said cannabis plant, or a plant part, tissue, or cell thereof of claim 75, wherein the inflorescence comprises a combined terpene oil content greater than about 1.5% by weight.

77. The cannabis plant, or an asexual clone of said cannabis plant, or a plant part, tissue, or cell thereof of claim 75, wherein the inflorescence comprises a combined terpene oil content greater than about 2.0% by weight.

78. The cannabis plant, or an asexual clone of said cannabis plant, or a plant part, tissue, or cell thereof of claim 74, wherein the inflorescence comprises a CBG max content of at least 3% by weight as measured by HPLC and calculated based on dry weight of the inflorescence.

79. The cannabis plant, or an asexual clone of said cannabis plant, or a plant part, tissue, or cell thereof of claim 74, wherein the inflorescence comprises a CBG max content of at least 4% by weight as measured by HPLC and calculated based on dry weight of the inflorescence.

80. The cannabis plant, or an asexual clone of said cannabis plant, or a plant part, tissue, or cell thereof of claim 74, wherein the inflorescence comprises a CBG max content of at least 5% by weight as measured by HPLC and calculated based on dry weight of the inflorescence.

81. The cannabis plant, or an asexual clone of said cannabis plant, or a plant part, tissue, or cell thereof of claim 73, wherein the tetrahydrocannabinol (THC max) and cannabidiol (CBD max) combined content is at least 7.5% by weight as measured by HPLC and calculated based on dry weight of the inflorescence.

82. The cannabis plant, or an asexual clone of said cannabis plant, or a plant part, tissue, or cell thereof of claim 73, wherein the tetrahydrocannabinol (THC max) and cannabidiol (CBD max) combined content is at least 10.0% by weight as measured by HPLC and calculated based on dry weight of the inflorescence.

83. The cannabis plant, or an asexual clone of said cannabis plant, or a plant part, tissue, or cell thereof of claim 73, wherein the tetrahydrocannabinol (THC max) and cannabidiol (CBD max) combined content is at least 12.5% by weight as measured by HPLC and calculated based on dry weight of the inflorescence.

84. A composition comprising:
a) a cannabigerol (CBG max) content of at least 20% by weight, and
b) a non-CBG max cannabinoid content of at least 30.0% by weight,
wherein the contents of all cannabinoids are measured by high performance liquid chromatography (HPLC) and calculated based on weight of the composition.

85. The composition of claim 84, wherein the composition comprises a terpene oil content greater than about 5% by weight;
wherein the terpene oil content is the additive content of terpinolene, alpha phellandrene, beta ocimene, carene, limonene, gamma terpinene, alpha pinene, alpha terpinene, beta pinene, fenchol, camphene, alpha terpineol, alpha humulene, beta caryophyllene, linalool, caryophyllene oxide, and myrcene as measured by GC-FID and calculated based on weight of the composition.

86. The composition of claim 85, wherein the composition comprises a terpene oil content greater than about 8% by weight.

87. The composition of claim 85, wherein the composition comprises a terpene oil content greater than about 10.0% by weight.

88. The composition of claim 4, wherein the composition comprises a CBG max content of at least 30% by weight as measured by HPLC and calculated based on weight of the composition.

89. The composition of claim 84, wherein the composition comprises a CBG max content of at least 40% by weight as measured by HPLC and calculated based on weight of the composition.

90. The composition of claim 84, wherein the composition comprises a CBG max content of at least 50% by weight as measured by HPLC and calculated based on dry weight of the composition.

91. The composition of claim 84, wherein the composition comprises a CBG max content of at least 60% by weight as measured by HPLC and calculated based on dry weight of the composition.

92. The composition of claim 84, wherein the composition comprises a CBG max content of at least 70% by weight as measured by HPLC and calculated based on dry weight of the composition.
